SEQUENCE LISTING (193 sequences)

SEQ ID NO: 1 - RNA, 23 nt, synthetic construct
  source 1..23: mol_type = other RNA
  misc_feature 1..23: note = GEiGS mature sRNA sequence (guide)
ttcagtgtagtacaagagataga

SEQ ID NO: 2 - RNA, 23 nt, synthetic construct
  source 1..23: mol_type = other RNA
  misc_feature 1..23: note = GEiGS mature sRNA sequence (guide)
tttcaattctctctccattcttc

SEQ ID NO: 3 - RNA, 22 nt, synthetic construct
  source 1..22: mol_type = other RNA
  misc_feature 1..22: note = GEiGS mature sRNA sequence (guide)
ttcagtaagtcaacttcaatgt

SEQ ID NO: 4 - RNA, 23 nt, synthetic construct
  source 1..23: mol_type = other RNA
  misc_feature 1..23: note = GEiGS mature sRNA sequence (guide)
ttcagtgtagtacaagagataga

SEQ ID NO: 5 - RNA, 23 nt, synthetic construct
  source 1..23: mol_type = other RNA
  misc_feature 1..23: note = GEiGS mature sRNA sequence (guide)
taaattttcccccaaattctaag

SEQ ID NO: 6 - RNA, 22 nt, synthetic construct
  source 1..22: mol_type = other RNA
  misc_feature 1..22: note = GEiGS mature sRNA sequence (guide)
tatatgacaaaatgtttcattc

SEQ ID NO: 7 - RNA, 23 nt, synthetic construct
  source 1..23: mol_type = other RNA
  misc_feature 1..23: note = GEiGS mature sRNA sequence (guide)
tactttatcaaatgtataagaag

SEQ ID NO: 8 - RNA, 23 nt, synthetic construct
  source 1..23: mol_type = other RNA
  misc_feature 1..23: note = GEiGS mature sRNA sequence (guide)
tagaattataaagaagatcatgt

SEQ ID NO: 9 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 10 - RNA, 23 nt, synthetic construct
  source 1..23: mol_type = other RNA
  misc_feature 1..23: note = GEiGS mature sRNA sequence (guide)
tgttaacattattataaccctac

SEQ ID NO: 11 - RNA, 23 nt, synthetic construct
  source 1..23: mol_type = other RNA
  misc_feature 1..23: note = GEiGS mature sRNA sequence (guide)
ttatatttctaaattttccccca

SEQ ID NO: 12 - RNA, 22 nt, synthetic construct
  source 1..22: mol_type = other RNA
  misc_feature 1..22: note = GEiGS mature sRNA sequence (guide)
taacattattataaccctacat

SEQ ID NO: 13 - RNA, 22 nt, synthetic construct
  source 1..22: mol_type = other RNA
  misc_feature 1..22: note = GEiGS mature sRNA sequence (guide)
tattataaccctacattttgtg

SEQ ID NO: 14 - RNA, 22 nt, synthetic construct
  source 1..22: mol_type = other RNA
  misc_feature 1..22: note = GEiGS mature sRNA sequence (guide)
ttaacttatgcacgcttaacta

SEQ ID NO: 15 - RNA, 22 nt, synthetic construct
  source 1..22: mol_type = other RNA
  misc_feature 1..22: note = GEiGS mature sRNA sequence (guide)
taactatcttaacaagctttga

SEQ ID NO: 16 - RNA, 23 nt, synthetic construct
  source 1..23: mol_type = other RNA
  misc_feature 1..23: note = GEiGS mature sRNA sequence (guide)
ttactttatcaaatgtataagaa

SEQ ID NO: 17 - RNA, 22 nt, synthetic construct
  source 1..22: mol_type = other RNA
  misc_feature 1..22: note = GEiGS mature sRNA sequence (guide)
tttatcaaatgtataagaagta

SEQ ID NO: 18 - RNA, 92 nt, synthetic construct
  source 1..92: mol_type = other RNA
  misc_feature 1..92: note = GEiGS solution sequence (pre-miRNA hairpin)
cggctggacagcgggttcagtgtagtacaagagatagattgtctccagagcattctctatctgctgactacactgcccctgctctcctgcct

SEQ ID NO: 19 - RNA, 69 nt, synthetic construct
  source 1..69: mol_type = other RNA
  misc_feature 1..69: note = GEiGS solution sequence (pre-miRNA hairpin)
ccacaagattggagagagtattgaaactttgaaactaaagaagtttcaattctctctccattcttctgg

SEQ ID NO: 20 - RNA, 68 nt, synthetic construct
  source 1..68: mol_type = other RNA
  misc_feature 1..68: note = GEiGS solution sequence (pre-miRNA hairpin)
cctacattttagttgacttactgcagctgtgtgaaacaaaagtttcagtaagtcaacttcaatgtagg

SEQ ID NO: 21 - RNA, 80 nt, synthetic construct
  source 1..80: mol_type = other RNA
  misc_feature 1..80: note = GEiGS solution sequence (pre-miRNA hairpin)
ctgggggctcttcagtgtagtacaagagatagatgtgattacccaactcttctctgtacttacactgcccgagcccccgg

SEQ ID NO: 22 - RNA, 81 nt, synthetic construct
  source 1..81: mol_type = other RNA
  misc_feature 1..81: note = GEiGS solution sequence (pre-miRNA hairpin)
ccttggccatgttaaattttcccccaaattctaagctttttgagatcttgaattagggggaaaattctaacattaccatgg

SEQ ID NO: 23 - RNA, 82 nt, synthetic construct
  source 1..82: mol_type = other RNA
  misc_feature 1..82: note = GEiGS solution sequence (pre-miRNA hairpin)
cctgccggggctatatgacaaaatgtttcattcgtggtcctctccgtgcgaccaaacattgtgtcatagctgctccagcagg

SEQ ID NO: 24 - RNA, 92 nt, synthetic construct
  source 1..92: mol_type = other RNA
  misc_feature 1..92: note = GEiGS solution sequence (pre-miRNA hairpin)
cggctggacagcgggtactttatcaaatgtataagaagttgtctccagagcattccttcttagccattgataaagcccctgctctcctgcct

SEQ ID NO: 25 - RNA, 68 nt, synthetic construct
  source 1..68: mol_type = other RNA
  misc_feature 1..68: note = GEiGS solution sequence (pre-miRNA hairpin)
cccactgacttcttaataattcttgggtgcggttgggagtctctagaattataaagaagatcatgtgg

SEQ ID NO: 26 - RNA, 71 nt, synthetic construct
  source 1..71: mol_type = other RNA
  misc_feature 1..71: note = GEiGS solution sequence (pre-miRNA hairpin)
gtagcactaacaaatttccaataatcctgttgtttagttatacagattattggaaatttgtaaagtactgc

SEQ ID NO: 27 - RNA, 72 nt, synthetic construct
  source 1..72: mol_type = other RNA
  misc_feature 1..72: note = GEiGS solution sequence (pre-miRNA hairpin)
tgtcgggtgttaacattattataaccctactgttgaatctcatggagggtcataatatgttaacatctgaca

SEQ ID NO: 28 - RNA, 69 nt, synthetic construct
  source 1..69: mol_type = other RNA
  misc_feature 1..69: note = GEiGS solution sequence (pre-miRNA hairpin)
ccacggggtaaaatttagtaatataactttgaaactaaagaagttatatttctaaattttcccccatgg

SEQ ID NO: 29 - RNA, 68 nt, synthetic construct
  source 1..68: mol_type = other RNA
  misc_feature 1..68: note = GEiGS solution sequence (pre-miRNA hairpin)
cctatgtattgttataataatgtcagctgtgtgaaacaaaagttaacattattataaccctacatagg

SEQ ID NO: 30 - RNA, 75 nt, synthetic construct
  source 1..75: mol_type = other RNA
  misc_feature 1..75: note = GEiGS solution sequence (pre-miRNA hairpin)
tgttgtcaaaatgtacagtatataatattgatgagtatcataggagaaatattataaccctacattttgtgaacc

SEQ ID NO: 31 - RNA, 83 nt, synthetic construct
  source 1..83: mol_type = other RNA
  misc_feature 1..83: note = GEiGS solution sequence (pre-miRNA hairpin)
tcatgctgtggcagttcagcgtgaataagtttcaattgtctgaaagaaaattaacttatgcacgcttaactattacggtttga

SEQ ID NO: 32 - RNA, 98 nt, synthetic construct
  source 1..98: mol_type = other RNA
  misc_feature 1..98: note = GEiGS solution sequence (pre-miRNA hairpin)
ttgggcaaggtaactatcttaacaagctttgagtcttactgaaggtttcctggaaaccacgcacatcaaagctgtataaaatagaaccttactcggtc

SEQ ID NO: 33 - RNA, 80 nt, synthetic construct
  source 1..80: mol_type = other RNA
  misc_feature 1..80: note = GEiGS solution sequence (pre-miRNA hairpin)
ctgggggctcttactttatcaaatgtataagaatgtgattacccaacttctataatttgtataaagtcccgagcccccgg

SEQ ID NO: 34 - RNA, 70 nt, synthetic construct
  source 1..70: mol_type = other RNA
  misc_feature 1..70: note = GEiGS solution sequence (pre-miRNA hairpin)
ggcacctttatcaaatgtataagaagtagggccttcgctactcacatcctattgatttgataaccgtgtc

SEQ ID NO: 35 - DNA, 274 bp, synthetic construct
  source 1..274: mol_type = other DNA
  misc_feature 1..274: note = hsa-mir-302a_11_B2M-exon2-2
gccagcacaccttttgttacaaaatttttttgttattgtgttttaaggttactaagcttgttacaggttaaaggattctaactttttccaagactgggctccccacaagattggagagagtattgaaactttgaaactaaagaagtttcaattctctctccattcttctggtaagtcttccttttacatttttattatttttttagaaaataactttattgtattgaccgcagctcatatatttaagctttattttgtatttttacatctgtta

SEQ ID NO: 36 - DNA, 274 bp, synthetic construct
  source 1..274: mol_type = other DNA
  misc_feature 1..274: note = hsa-mir-302a_35_B2M-exon4
gccagcacaccttttgttacaaaatttttttgttattgtgttttaaggttactaagcttgttacaggttaaaggattctaactttttccaagactgggctccccacggggtaaaatttagtaatataactttgaaactaaagaagttatatttctaaattttcccccatggtaagtcttccttttacatttttattatttttttagaaaataactttattgtattgaccgcagctcatatatttaagctttattttgtatttttacatctgtta

SEQ ID NO: 37 - DNA, 216 bp, synthetic construct
  source 1..216: mol_type = other DNA
  misc_feature 1..216: note = hsa-mir-302c_12_B2M-exon2-2
tttacttctccaaaatagaacacgctaacctcatttgaagggatcccctacattttagttgacttactgcagctgtgtgaaacaaaagtttcagtaagtcaacttcaatgtaggtgtctccaagccagcacaccttttgttacaaaatttttttgttattgtgttttaaggttactaagcttgttacaggttaaaggattctaactttttccaaga

SEQ ID NO: 38 - DNA, 216 bp, synthetic construct
  source 1..216: mol_type = other DNA
  misc_feature 1..216: note = hsa-mir-302c_36_B2M-exon4
tttacttctccaaaatagaacacgctaacctcatttgaagggatcccctatgtattgttataataatgtcagctgtgtgaaacaaaagttaacattattataaccctacataggtgtctccaagccagcacaccttttgttacaaaatttttttgttattgtgttttaaggttactaagcttgttacaggttaaaggattctaactttttccaaga

SEQ ID NO: 39 - DNA, 200 bp, synthetic construct
  source 1..200: mol_type = other DNA
  misc_feature 1..200: note = hsa-mir-20a_29_B2M-exon4
gaatgatttttactaattttgtgtacttttattgtgtcgatgtagaatctgcctggtctatctgatgtgacagcttctgtagcactaacaaatttccaataatcctgttgtttagttatacagattattggaaatttgtaaagtactgctagctgtagaactccagcttcggcctgtcgcccaatcaaactgtcctgtta

SEQ ID NO: 40 - DNA, 328 bp, synthetic construct
  source 1..328: mol_type = other DNA
  misc_feature 1..328: note = hsa-mir-200c_27_B2M-exon4
agcagggctcaccaggaagtgtccccagggactcgggtggtggggggatgggagccagggatctgcagcttttccgcagggatcctgggcctgaagctgcctgacccaaggtgggcgggctgggcgggggcccactgacttcttaataattcttgggtgcggttgggagtctctagaattataaagaagatcatgtggcccctgtccctgtgtcagcaacatccatcgcctcaggtccccagcccttagctggctgcagccccctccccacttcccacgcaccccggaagcccctcgtcttgagctgagagcgttgcacaaggggtgg

SEQ ID NO: 41 - DNA, 332 bp, synthetic construct
  source 1..332: mol_type = other DNA
  misc_feature 1..332: note = hsa-mir-21_30_B2M-exon4
gttcgatcttaacaggccagaaatgcctgggtttttttggtttgtttttgtttttgtttttttatcaaatcctgcctgactgtctgcttgttttgcctaccatcgtgacatctccatggctgtaccaccttgtcgggtgttaacattattataaccctactgttgaatctcatggagggtcataatatgttaacatctgacattttggtatctttcatctgaccatccatatccaatgttctcatttaaacattacccagcatcattgtttataatcagaaactctggtccttctgtctggtggcacttagagtcttttgtgccataatgca

SEQ ID NO: 42 - DNA, 275 bp, synthetic construct
  source 1..275: mol_type = other DNA
  misc_feature 1..275: note = hsa-mir-363_41_B2M-exon4
tatcgtcttattttaacttttaaaagccgtaagttctgatatttagtcattgtaaaatgatctgttttgctgttgtcaaaatgtacagtatataatattgatgagtatcataggagaaatattataaccctacattttgtgaaccgcaggacctttgttggcgacattcctgatcagcgctacagtaagctagatggtaaaaaatcctttctctagttgctgctattaaaaaataatttgaaatatatttggatttctaaaaactatgatagcta

SEQ ID NO: 43 - DNA, 293 bp, synthetic construct
  source 1..293: mol_type = other DNA
  misc_feature 1..293: note = hsa-mir-93_19_B2M-exon2-2
aggccttttccccacttcttaaccttcactgagagggtggttggggtctgtttcactccatgtgtcctagatcctgtgctacagaccttcctttctgtcctcccgtcttggacctcagtcctgggggctcttcagtgtagtacaagagatagatgtgattacccaactcttctctgtacttacactgcccgagcccccgggacacgttctctctgccaattgtcttcttggctgagctccccaagctccatctgtcatgctggggagcccagtggcgttcaaaagggtctggt

SEQ ID NO: 44 - DNA, 293 bp, synthetic construct
  source 1..293: mol_type = other DNA
  misc_feature 1..293: note = hsa-mir-93_49_B2M-exon4
aggccttttccccacttcttaaccttcactgagagggtggttggggtctgtttcactccatgtgtcctagatcctgtgctacagaccttcctttctgtcctcccgtcttggacctcagtcctgggggctcttactttatcaaatgtataagaatgtgattacccaacttctataatttgtataaagtcccgagcccccgggacacgttctctctgccaattgtcttcttggctgagctccccaagctccatctgtcatgctggggagcccagtggcgttcaaaagggtctggt

SEQ ID NO: 45 - DNA, 287 bp, synthetic construct
  source 1..287: mol_type = other DNA
  misc_feature 1..287: note = hsa-mir-106a_20_B2M-exon4
tttacttgtttagtttctaatatgtgtgtgtttgttttgttgttttaaccaggtgagtctgcatggatctgtgaggacggaaaagaagagctcctggaagacttaaaattttgctacaggaataggccttggccatgttaaattttcccccaaattctaagctttttgagatcttgaattagggggaaaattctaacattaccatggtgatttagtcaatggctactgagaactgtagtttgtgcataattaagtagttgatgcttttgagctgcttcttataatgt

SEQ ID NO: 46 - DNA, 342 bp, synthetic construct
  source 1..342: mol_type = other DNA
  misc_feature 1..342: note = hsa-mir-106b_21_B2M-exon4
tgctggctatcctgcgcctttccactgctctggtaagtgcccaaattgctggagggccatctgttttgacccttaaaggggtagctccttaccgtgctctcattgccgcctccccacctcccgctccagccctgccggggctatatgacaaaatgtttcattcgtggtcctctccgtgcgaccaaacattgtgtcatagctgctccagcagggcacgcacagcgtccgtggagggaaaggccttttccccacttcttaaccttcactgagagggtggttggggtctgtttcactccatgtgtcctagatcctgtgctacagaccttcctttctgtcctcccg

SEQ ID NO: 47 - DNA, 352 bp, synthetic construct
  source 1..352: mol_type = other DNA
  misc_feature 1..352: note = hsa-mir-191_8_B2M-exon2-2
gtcttgttccctctagactccgtttcacaacctactcccgggtcttcctcctgggactggggatgggcgggttctgatgtggccccagggcgagtgacctgggggcaggagctcccccgccccccgccaacggctggacagcgggttcagtgtagtacaagagatagattgtctccagagcattctctatctgctgactacactgcccctgctctcctgcctgagcagcgccctggcccagatggggtgcccctgacccccagacatactttactgagctgcttgggtctcagttcctctcagttgcgccctcaggctggaggtgatgggtgtagacgtgggagagccgagg

SEQ ID NO: 48 - DNA, 352 bp, synthetic construct
  source 1..352: mol_type = other DNA
  misc_feature 1..352: note = hsa-mir-191_26_B2M-exon4
gtcttgttccctctagactccgtttcacaacctactcccgggtcttcctcctgggactggggatgggcgggttctgatgtggccccagggcgagtgacctgggggcaggagctcccccgccccccgccaacggctggacagcgggtactttatcaaatgtataagaagttgtctccagagcattccttcttagccattgataaagcccctgctctcctgcctgagcagcgccctggcccagatggggtgcccctgacccccagacatactttactgagctgcttgggtctcagttcctctcagttgcgccctcaggctggaggtgatgggtgtagacgtgggagagccgagg

SEQ ID NO: 49 - DNA, 358 bp, synthetic construct
  source 1..358: mol_type = other DNA
  misc_feature 1..358: note = hsa-mir-744_47_B2M-exon4
cattaacatgttttaaacttcaggcccttctactgccaaggtgagttcaggctgggcggctgcacccctgggagcagggcagtgctgcactgagccaggcgggagctggaagaagacgcagcacactgggttgggcaaggtaactatcttaacaagctttgagtcttactgaaggtttcctggaaaccacgcacatcaaagctgtataaaatagaaccttactcggtcctgaccggctcggcttctgtttgtttatttcatctctactcagtactgccctgttccctggttttaaagttgtactgaaatgcataccttgtgataatgttgtcacattttgtccttattgtgtcgtgcc

SEQ ID NO: 50 - DNA, 343 bp, synthetic construct
  source 1..343: mol_type = other DNA
  misc_feature 1..343: note = hsa-mir-518b_43_B2M-exon4
tttatgttctggattccagaaaacatgcaaacagggcaaataaatgcatctttattttgtgtccattttaacctggtcaaggaaaattccaacagcaacatcaaaaaaccagtgttggagcaagaatatgtcatgctgtggcagttcagcgtgaataagtttcaattgtctgaaagaaaattaacttatgcacgcttaactattacggtttgagtaaagcagcgttgaagttgatgctgatcttggtaatacatttgcagagcgtgcttatcatcagacgtggacgatggtggggttctgttttggttttgtttttttctaagacagggtctccgttgcccag

SEQ ID NO: 51 - DNA, 294 bp, synthetic construct
  source 1..294: mol_type = other DNA
  misc_feature 1..294: note = hsa-mir-99b_50_B2M-exon4
cttctgggttctttagggaggagggggatgagagccttgactccagggtccctgatgaggaaggggctgagggcctggactcctgggttccttggggaggaggggccgggggcccggactcctgggtcctggcacctttatcaaatgtataagaagtagggccttcgctactcacatcctattgatttgataaccgtgtcgggggctcaccatcgcggctggggcctccccggccctccccctcatccctggtcctcctggtccctgtctgtctgtctgtcgggtctgtccacc

SEQ ID NO: 52 - RNA, 22 nt, synthetic construct
  source 1..22: mol_type = other RNA
  misc_feature 1..22: note = GEiGS mature sRNA sequence (guide)
ttaactaaacaacaacaaacag

SEQ ID NO: 53 - RNA, 69 nt, synthetic construct
  source 1..69: mol_type = other RNA
  misc_feature 1..69: note = GEiGS solution sequence (pre-miRNA hairpin)
ccaaccctttaactaaacaacaacaaacaggaaacaaaattgtttgttgtggtttagttgtggggatgg

SEQ ID NO: 54 - RNA, 23 nt, synthetic construct
  source 1..23: mol_type = other RNA
  misc_feature 1..23: note = GEiGS mature sRNA sequence (guide)
tttacgtcgataaatcaggacac

SEQ ID NO: 55 - RNA, 68 nt, synthetic construct
  source 1..68: mol_type = other RNA
  misc_feature 1..68: note = GEiGS solution sequence (pre-miRNA hairpin)
cccgttccgatttaacgacgtaatgggtgcggttgggagtctctttacgtcgataaatcaggacacgg

SEQ ID NO: 56 - DNA, 274 bp, synthetic construct
  source 1..274: mol_type = other DNA
  misc_feature 1..274: note = hsa-mir-302a_23_None (negative control)
gccagcacaccttttgttacaaaatttttttgttattgtgttttaaggttactaagcttgttacaggttaaaggattctaactttttccaagactgggctccccaaccctttaactaaacaacaacaaacaggaaacaaaattgtttgttgtggtttagttgtggggatggtaagtcttccttttacatttttattatttttttagaaaataactttattgtattgaccgcagctcatatatttaagctttattttgtatttttacatctgtta

SEQ ID NO: 57 - DNA, 328 bp, synthetic construct
  source 1..328: mol_type = other DNA
  misc_feature 1..328: note = hsa-mir-200c_13_None (negative control)
agcagggctcaccaggaagtgtccccagggactcgggtggtggggggatgggagccagggatctgcagcttttccgcagggatcctgggcctgaagctgcctgacccaaggtgggcgggctgggcgggggcccgttccgatttaacgacgtaatgggtgcggttgggagtctctttacgtcgataaatcaggacacggcccctgtccctgtgtcagcaacatccatcgcctcaggtccccagcccttagctggctgcagccccctccccacttcccacgcaccccggaagcccctcgtcttgagctgagagcgttgcacaaggggtgg

SEQ ID NO: 58 - RNA, 20 nt, synthetic construct
  source 1..20: mol_type = other RNA
  misc_feature 1..20: note = gRNA sequence
gcactgtaagcacttttaca

SEQ ID NO: 59 - DNA, 632 bp, synthetic construct
  source 1..632: mol_type = other DNA
  misc_feature 1..632: note = HDR template sequence
tatgagcactttggtactactaggacccaatcttatcctctgtacacaactgtacactgcacagtccccaccatctgttcccaacaaacaataaaaaggcaaaaaacaaaccactgaaatgtgtcctattcaaattactactcaaggcatacttttaaaatgctgtatattaggcagcctgtgccagaaggggcatttagggcagtagattctaagctgcttcactaactgcactagatgcaccttaacacaagagacacattataagaagcagctcaaaagcatcaactacttaattatgcacaaactacagttctcagtagccattgactaaatcaccatggtaatgttagaattttccccctaattcaagatctcaaaaagcttagaatttgggggaaaatttaacatggccaaggcctattcctgtagcaaaattttaagtcttccaggagctcttcttttccgtcctcacagatccatgcagactcacctggttaaaacaacaaaacaaacacacacatattagaaactaaacaagtaaactactagttaatattcctgaaacaggctttcctttggcttacagagtctaagctaggttttgtggtttcaaccaaatcctgagaaat

SEQ ID NO: 60 - RNA, 20 nt, synthetic construct
  source 1..20: mol_type = other RNA
  misc_feature 1..20: note = gRNA sequence
cactaaagtgcttatagtgc

SEQ ID NO: 61 - DNA, 750 bp, synthetic construct
  source 1..750: mol_type = other DNA
  misc_feature 1..750: note = HDR template sequence
agggcctgctgatgttgagtgctttttgttctaaggtgcatctagtgcagatagtgaagtagattagcatctactgccctaagtgctccttctggcataagaagttatgtattcatccaataattcaagccaagcaagtatataggtgttttaatagtttttgtttgcagtcctctgttagttttgcatagttgcactacaagaagaatgtagttgtgcaaatctatgcaaaactgatggtggcctgctatttccttcaaatgaatgatttttactaattttgtgtacttttattgtgtcgatgtagaatctgcctggtctatctgatgtgacagcttctgtagcactaacaaatttccaataatcctgttgtttagttatacagattattggaaatttgtaaagtactgctagctgtagaactccagcttcggcctgtcgcccaatcaaactgtcctgttactgaacactgttctatggttagttttgcaggtttgcatccagctgtgtgatattctgctgtgcaaatccatgcaaaactgactgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatacatgaa

SEQ ID NO: 62 - RNA, 20 nt, synthetic construct
  source 1..20: mol_type = other RNA
  misc_feature 1..20: note = gRNA sequence
atgttgactgttgaatctca

SEQ ID NO: 63 - DNA, 765 bp, synthetic construct
  source 1..765: mol_type = other DNA
  misc_feature 1..765: note = HDR template sequence
agcatgtactctggtttcaacagacacaaatttatatgttaacccagttttcttgccgttctgtaagtgttttattcttagtgtgatttttttccattgggatgtttttgattgaacttgttcattttgttttgcttgggaggaaaataaacaattttacttttttcctttaggagcattatgagcattatgtcagaatagaatagaattggggttcgatcttaacaggccagaaatgcctgggtttttttggtttgtttttgtttttgtttttttatcaaatcctgcctgactgtctgcttgttttgcctaccatcgtgacatctccatggctgtaccaccttgtcgggtgttaacattattataaccctactgttgaatctcatggagggtcataatatgttaacatctgacattttggtatctttcatctgaccatccatatccaatgttctcatttaaacattacccagcatcattgtttataatcagaaactctggtccttctgtctggtggcacttagagtcttttgtgccataatgcagcagtatggagggaggattttatggagaaatggggatagtcttcatgaccacaaataaataaaggaaaactaagctgcattgtgggttttgaaaaggttattatacttcttaacaattctttttttcagggacttttctagctgtatgactgttacttgaccttctttgaaaagcattcccaaaatgctctattttagatagattaacattaaccaacat

SEQ ID NO: 64 - RNA, 22 nt, synthetic construct
  source 1..22: mol_type = other RNA
  misc_feature 1..22: note = GEiGS mature sRNA sequence (guide)
taaacctgaatctttggagtac

SEQ ID NO: 65 - RNA, 22 nt, synthetic construct
  source 1..22: mol_type = other RNA
  misc_feature 1..22: note = GEiGS mature sRNA sequence (guide)
taaacctgaatctttggagtac

SEQ ID NO: 66 - RNA, 22 nt, synthetic construct
  source 1..22: mol_type = other RNA
  misc_feature 1..22: note = GEiGS mature sRNA sequence (guide)
taaacctgaatctttggagtac

SEQ ID NO: 67 - RNA, 23 nt, synthetic construct
  source 1..23: mol_type = other RNA
  misc_feature 1..23: note = GEiGS mature sRNA sequence (guide)
ttcagtaagtcaacttcaatgtc

SEQ ID NO: 68 - RNA, 22 nt, synthetic construct
  source 1..22: mol_type = other RNA
  misc_feature 1..22: note = GEiGS mature sRNA sequence (guide)
tttttcaattctctctccattc

SEQ ID NO: 69 - RNA, 22 nt, synthetic construct
  source 1..22: mol_type = other RNA
  misc_feature 1..22: note = GEiGS mature sRNA sequence (guide)
tttttcaattctctctccattc

SEQ ID NO: 70 - RNA, 24 nt, synthetic construct
  source 1..24: mol_type = other RNA
  misc_feature 1..24: note = GEiGS mature sRNA sequence (guide)
ttcttcagtaagtcaacttcaatg

SEQ ID NO: 71 - RNA, 22 nt, synthetic construct
  source 1..22: mol_type = other RNA
  misc_feature 1..22: note = GEiGS mature sRNA sequence (guide)
ttcagtgtagtacaagagatag

SEQ ID NO: 72 - RNA, 22 nt, synthetic construct
  source 1..22: mol_type = other RNA
  misc_feature 1..22: note = GEiGS mature sRNA sequence (guide)
ttcagtaagtcaacttcaatgt

SEQ ID NO: 73 - RNA, 22 nt, synthetic construct
  source 1..22: mol_type = other RNA
  misc_feature 1..22: note = GEiGS mature sRNA sequence (guide)
tttcaattctctctccattctt

SEQ ID NO: 74 - RNA, 23 nt, synthetic construct
  source 1..23: mol_type = other RNA
  misc_feature 1..23: note = GEiGS mature sRNA sequence (guide)
ttcagtaagtcaacttcaatgtc

SEQ ID NO: 75 - RNA, 22 nt, synthetic construct
  source 1..22: mol_type = other RNA
  misc_feature 1..22: note = GEiGS mature sRNA sequence (guide)
tttcaattctctctccattctt

SEQ ID NO: 76 - RNA, 22 nt, synthetic construct
  source 1..22: mol_type = other RNA
  misc_feature 1..22: note = GEiGS mature sRNA sequence (guide)
ttcttcagtaagtcaacttcaa

SEQ ID NO: 77 - RNA, 22 nt, synthetic construct
  source 1..22: mol_type = other RNA
  misc_feature 1..22: note = GEiGS mature sRNA sequence (guide)
tttcaattctctctccattctt

SEQ ID NO: 78 - RNA, 22 nt, synthetic construct
  source 1..22: mol_type = other RNA
  misc_feature 1..22: note = GEiGS mature sRNA sequence (guide)
ttcagtgtagtacaagagatag

SEQ ID NO: 79 - RNA, 22 nt, synthetic construct
  source 1..22: mol_type = other RNA
  misc_feature 1..22: note = GEiGS mature sRNA sequence (guide)
ttaactatcttaacaagctttg

SEQ ID NO: 80 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 81 - RNA, 22 nt, synthetic construct
  source 1..22: mol_type = other RNA
  misc_feature 1..22: note = GEiGS mature sRNA sequence (guide)
taacattattataaccctacat

SEQ ID NO: 82 - RNA, 24 nt, synthetic construct
  source 1..24: mol_type = other RNA
  misc_feature 1..24: note = GEiGS mature sRNA sequence (guide)
tatcaatattaaaaagcaagcaag

SEQ ID NO: 83 - RNA, 22 nt, synthetic construct
  source 1..22: mol_type = other RNA
  misc_feature 1..22: note = GEiGS mature sRNA sequence (guide)
ttcattcattataacaaatttc

SEQ ID NO: 84 - RNA, 22 nt, synthetic construct
  source 1..22: mol_type = other RNA
  misc_feature 1..22: note = GEiGS mature sRNA sequence (guide)
ttaacattattataaccctaca

SEQ ID NO: 85 - RNA, 23 nt, synthetic construct
  source 1..23: mol_type = other RNA
  misc_feature 1..23: note = GEiGS mature sRNA sequence (guide)
taacattattataaccctacatt

SEQ ID NO: 86 - RNA, 24 nt, synthetic construct
  source 1..24: mol_type = other RNA
  misc_feature 1..24: note = GEiGS mature sRNA sequence (guide)
tgttaacattattataaccctaca

SEQ ID NO: 87 - RNA, 22 nt, synthetic construct
  source 1..22: mol_type = other RNA
  misc_feature 1..22: note = GEiGS mature sRNA sequence (guide)
tataaagaagatcatgtccatg

SEQ ID NO: 88 - RNA, 24 nt, synthetic construct
  source 1..24: mol_type = other RNA
  misc_feature 1..24: note = GEiGS mature sRNA sequence (guide)
tttatgatttatttaacttgtgga

SEQ ID NO: 89 - RNA, 22 nt, synthetic construct
  source 1..22: mol_type = other RNA
  misc_feature 1..22: note = GEiGS mature sRNA sequence (guide)
ttattataaccctacattttgt

SEQ ID NO: 90 - RNA, 23 nt, synthetic construct
  source 1..23: mol_type = other RNA
  misc_feature 1..23: note = GEiGS mature sRNA sequence (guide)
taacattattataaccctacatt

SEQ ID NO: 91 - RNA, 22 nt, synthetic construct
  source 1..22: mol_type = other RNA
  misc_feature 1..22: note = GEiGS mature sRNA sequence (guide)
ttattataaccctacattttgt

SEQ ID NO: 92 - RNA, 23 nt, synthetic construct
  source 1..23: mol_type = other RNA
  misc_feature 1..23: note = GEiGS mature sRNA sequence (guide)
taacttatgcacgcttaactatc

SEQ ID NO: 93 - RNA, 22 nt, synthetic construct
  source 1..22: mol_type = other RNA
  misc_feature 1..22: note = GEiGS mature sRNA sequence (guide)
taacattattataaccctacat

SEQ ID NO: 94 - RNA, 22 nt, synthetic construct
  source 1..22: mol_type = other RNA
  misc_feature 1..22: note = GEiGS mature sRNA sequence (guide)
tttatgatttatttaacttgtg

SEQ ID NO: 95 - RNA, 22 nt, synthetic construct
  source 1..22: mol_type = other RNA
  misc_feature 1..22: note = GEiGS mature sRNA sequence (guide)
ttcattataacaaatttccaat

SEQ ID NO: 96 - RNA, 22 nt, synthetic construct
  source 1..22: mol_type = other RNA
  misc_feature 1..22: note = GEiGS mature sRNA sequence (guide)
ttttatgatttatttaacttgt

SEQ ID NO: 97-102 - skipped (6 entries)

SEQ ID NO: 103 - RNA, 23 nt, synthetic construct
  source 1..23: mol_type = other RNA
  misc_feature 1..23: note = GEiGS mature sRNA sequence (guide)
ttaacttatgcacgcttaactat

SEQ ID NO: 104 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 105 - RNA, 23 nt, synthetic construct
  source 1..23: mol_type = other RNA
  misc_feature 1..23: note = GEiGS mature sRNA sequence (guide)
ttaacttatgcacgcttaactat

SEQ ID NO: 106 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 107 - RNA, 23 nt, synthetic construct
  source 1..23: mol_type = other RNA
  misc_feature 1..23: note = GEiGS mature sRNA sequence (guide)
ttaacttatgcacgcttaactat

SEQ ID NO: 108 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 109 - RNA, 23 nt, synthetic construct
  source 1..23: mol_type = other RNA
  misc_feature 1..23: note = GEiGS mature sRNA sequence (guide)
tacaagagatagaaagaccagtc

SEQ ID NO: 110 - RNA, 23 nt, synthetic construct
  source 1..23: mol_type = other RNA
  misc_feature 1..23: note = GEiGS mature sRNA sequence (guide)
tacaagagatagaaagaccagtc

SEQ ID NO: 111 - RNA, 23 nt, synthetic construct
  source 1..23: mol_type = other RNA
  misc_feature 1..23: note = GEiGS mature sRNA sequence (guide)
tacaagagatagaaagaccagtc

SEQ ID NO: 112 - RNA, 22 nt, synthetic construct
  source 1..22: mol_type = other RNA
  misc_feature 1..22: note = GEiGS mature sRNA sequence (guide)
ttaacttatgcacgcttaacta

SEQ ID NO: 113 - RNA, 22 nt, synthetic construct
  source 1..22: mol_type = other RNA
  misc_feature 1..22: note = GEiGS mature sRNA sequence (guide)
taacaaatttccaataatcctg

SEQ ID NO: 114 - RNA, 22 nt, synthetic construct
  source 1..22: mol_type = other RNA
  misc_feature 1..22: note = GEiGS mature sRNA sequence (guide)
tacaagagatagaaagaccagt

SEQ ID NO: 115 - RNA, 22 nt, synthetic construct
  source 1..22: mol_type = other RNA
  misc_feature 1..22: note = GEiGS mature sRNA sequence (guide)
ttgactttccattctctgctgg

SEQ ID NO: 116 - RNA, 22 nt, synthetic construct
  source 1..22: mol_type = other RNA
  misc_feature 1..22: note = GEiGS mature sRNA sequence (guide)
ttgactttccattctctgctgg

SEQ ID NO: 117 - RNA, 22 nt, synthetic construct
  source 1..22: mol_type = other RNA
  misc_feature 1..22: note = GEiGS mature sRNA sequence (guide)
ttgactttccattctctgctgg

SEQ ID NO: 118 - RNA, 23 nt, synthetic construct
  source 1..23: mol_type = other RNA
  misc_feature 1..23: note = GEiGS mature sRNA sequence (guide)
ggcgtctcggtggcacgacttgt

SEQ ID NO: 119 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 120 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 121 - RNA, 23 nt, synthetic construct
  source 1..23: mol_type = other RNA
  misc_feature 1..23: note = GEiGS mature sRNA sequence (guide)
taacaaatttccaataatcctgt

SEQ ID NO: 122 - RNA, 23 nt, synthetic construct
  source 1..23: mol_type = other RNA
  misc_feature 1..23: note = GEiGS mature sRNA sequence (guide)
ttaacttatgcacgcttaactat

SEQ ID NO: 123 - RNA, 23 nt, synthetic construct
  source 1..23: mol_type = other RNA
  misc_feature 1..23: note = GEiGS mature sRNA sequence (guide)
ttaacttatgcacgcttaactat

SEQ ID NO: 124 - RNA, 23 nt, synthetic construct
  source 1..23: mol_type = other RNA
  misc_feature 1..23: note = GEiGS mature sRNA sequence (guide)
ttaacttatgcacgcttaactat

SEQ ID NO: 125 - RNA, 23 nt, synthetic construct
  source 1..23: mol_type = other RNA
  misc_feature 1..23: note = GEiGS mature sRNA sequence (guide)
tacaagagatagaaagaccagtc

SEQ ID NO: 126 - RNA, 23 nt, synthetic construct
  source 1..23: mol_type = other RNA
  misc_feature 1..23: note = GEiGS mature sRNA sequence (guide)
tacaagagatagaaagaccagtc

SEQ ID NO: 127 - RNA, 23 nt, synthetic construct
  source 1..23: mol_type = other RNA
  misc_feature 1..23: note = GEiGS mature sRNA sequence (guide)
tacaagagatagaaagaccagtc

SEQ ID NO: 128 - RNA, 72 nt, synthetic construct
  source 1..72: mol_type = other RNA
  misc_feature 1..72: note = GEiGS solution sequence (pre-miRNA hairpin)
ctcccctctactccaaattttcaggtctacccggagtcgggtaaacctgaatctttggagtacaaaaggatg

SEQ ID NO: 129 - RNA, 87 nt, synthetic construct
  source 1..87: mol_type = other RNA
  misc_feature 1..87: note = GEiGS solution sequence (pre-miRNA hairpin)
tctcaggctgtggtactccaaagattcaggttttctgtggtcagaaagaaaagtaaacctgaatctttggagtactaccgtttggga

SEQ ID NO: 130 - RNA, 98 nt, synthetic construct
  source 1..98: mol_type = other RNA
  misc_feature 1..98: note = GEiGS solution sequence (pre-miRNA hairpin)
acgaatggctatgcactgtactccaaagagagagtcaggtttacacatagactataattgtaaacctgaatctttggagtacagtgcacaacctacac

SEQ ID NO: 131 - RNA, 81 nt, synthetic construct
  source 1..81: mol_type = other RNA
  misc_feature 1..81: note = GEiGS solution sequence (pre-miRNA hairpin)
ccttggccatgtttcagtaagtcaacttcaatgtcctttttgagatgacttgaaattgacttactgcaaacattaccatgg

SEQ ID NO: 132 - RNA, 69 nt, synthetic construct
  source 1..69: mol_type = other RNA
  misc_feature 1..69: note = GEiGS solution sequence (pre-miRNA hairpin)
gctgctggggagagcgattgaaaaatagtgctcctggttgtttttcaattctctctccattccggcggc

SEQ ID NO: 133 - RNA, 98 nt, synthetic construct
  source 1..98: mol_type = other RNA
  misc_feature 1..98: note = GEiGS solution sequence (pre-miRNA hairpin)
ttgaggccttaaagtactgtttttcaattctctctccattctgctacagtcaagagactggagagtgatttgaaaactagaaatttaaggaaattcat

SEQ ID NO: 134 - RNA, 110 nt, synthetic construct
  source 1..110: mol_type = other RNA
  misc_feature 1..110: note = GEiGS solution sequence (pre-miRNA hairpin)
gagctgcttgcctccccccgttttcttcagtaagtcaacttcaatgggtgaggtaacaggatcccattgttgacgacctgaagactatggggcgaggactcagccggcac

SEQ ID NO: 135 - RNA, 73 nt, synthetic construct
  source 1..73: mol_type = other RNA
  misc_feature 1..73: note = GEiGS solution sequence (pre-miRNA hairpin)
ggccggcctatccttgtacgacacggaattgcttcctgtcacaattcagtgtagtacaagagatagaccgacc

SEQ ID NO: 136 - RNA, 77 nt, synthetic construct
  source 1..77: mol_type = other RNA
  misc_feature 1..77: note = GEiGS solution sequence (pre-miRNA hairpin)
ccgggacccagttcagtaagtcaacttcaatgtgtgtgctgtccacattgaagtccacttactgactcggggaccgg

SEQ ID NO: 137 - RNA, 72 nt, synthetic construct
  source 1..72: mol_type = other RNA
  misc_feature 1..72: note = GEiGS solution sequence (pre-miRNA hairpin)
ctcccctcagaatggagttagaattgcaacccggagtcgggtttcaattctctctccattcttaaaaggatg

SEQ ID NO: 138 - RNA, 94 nt, synthetic construct
  source 1..94: mol_type = other RNA
  misc_feature 1..94: note = GEiGS solution sequence (pre-miRNA hairpin)
tgttttgagcgggggttcagtaagtcaacttcaatgtcttgtcataaacacattgaaattgacttaccgaacctcctctcatttgctatattca

SEQ ID NO: 139 - RNA, 72 nt, synthetic construct
  source 1..72: mol_type = other RNA
  misc_feature 1..72: note = GEiGS solution sequence (pre-miRNA hairpin)
ggagctttcaattctctctccattctttttataatttcaaaaaaacaatgaagtgtgagaattgattgcttc

SEQ ID NO: 140 - RNA, 83 nt, synthetic construct
  source 1..83: mol_type = other RNA
  misc_feature 1..83: note = GEiGS solution sequence (pre-miRNA hairpin)
tcatgctgtggctgaacttgactaactgaagtcaattgtctgaaagaaaattcttcagtaagtcaacttcaattacggtttga

SEQ ID NO: 141 - RNA, 87 nt, synthetic construct
  source 1..87: mol_type = other RNA
  misc_feature 1..87: note = GEiGS solution sequence (pre-miRNA hairpin)
tctcaggctgtgaagaatggagagagaattgaatctgtggtcagaaagaaaagtttcaattctctctccattctttaccgtttggga

SEQ ID NO: 142 - RNA, 98 nt, synthetic construct
  source 1..98: mol_type = other RNA
  misc_feature 1..98: note = GEiGS solution sequence (pre-miRNA hairpin)
acgaatggctatgcactctatctcttgtagagagtacactgaacacatagactataattgttcagtgtagtacaagagatagagtgcacaacctacac

SEQ ID NO: 143 - RNA, 69 nt, synthetic construct
  source 1..69: mol_type = other RNA
  misc_feature 1..69: note = GEiGS solution sequence (pre-miRNA hairpin)
gctgctgcgcttgtcaaatagttaatagtgctcctggttgttaactatcttaacaagctttgcggcggc

SEQ ID NO: 144 - RNA, 149 nt, synthetic construct
  source 1..149: mol_type = other RNA
  misc_feature 1..149: note = GEiGS solution sequence (pre-miRNA hairpin)
catcaagacccagctgagtcactgtcactgcctaccaaactggattatagatatattgtcacgtctgtgttgccaatcgtaacaaatttccaataatcctgttagataggcggtcatgcatacgaattttcagctcttgttctggtgac

SEQ ID NO: 145 - RNA, 98 nt, synthetic construct
  source 1..98: mol_type = other RNA
  misc_feature 1..98: note = GEiGS solution sequence (pre-miRNA hairpin)
ttgaggccttaaagtactgtaacattattataaccctacattgctacagtcaagaatctagggttttattaatgttctagaaatttaaggaaattcat

SEQ ID NO: 146 - RNA, 110 nt, synthetic construct
  source 1..110: mol_type = other RNA
  misc_feature 1..110: note = GEiGS solution sequence (pre-miRNA hairpin)
gagctgcttgcctccccccgtttatcaatattaaaaagcaagcaagggtgaggtaacaggatcccttggcttttgactattgatctatggggcgaggactcagccggcac

SEQ ID NO: 147 - RNA, 110 nt, synthetic construct
  source 1..110: mol_type = other RNA
  misc_feature 1..110: note = GEiGS solution sequence (pre-miRNA hairpin)
aaagatcctcagacaatccatgtgcttctcttgttcattcattataacaaatttctctcatacccaacgaaatttgatataatgaatgttcaggaggcatggagctgaca

SEQ ID NO: 148 - RNA, 85 nt, synthetic construct
  source 1..85: mol_type = other RNA
  misc_feature 1..85: note = GEiGS solution sequence (pre-miRNA hairpin)
ggctgagccgcatgtagggttatataatgattaatatgtcctgacccagctattaacattattataaccctacatgccctctgcc

SEQ ID NO: 149 - RNA, 110 nt, synthetic construct
  source 1..110: mol_type = other RNA
  misc_feature 1..110: note = GEiGS solution sequence (pre-miRNA hairpin)
tgaacatccaggtctggggcataattagggttttaataatgtattttagtgttcgttaggcaactaacattattataaccctacattaggctacctggaaacatgttctc

SEQ ID NO: 150 - RNA, 110 nt, synthetic construct
  source 1..110: mol_type = other RNA
  misc_feature 1..110: note = GEiGS solution sequence (pre-miRNA hairpin)
gctgctggaaggtgtaggtaccctcaattagggttataataagttaaatcctgtctttcgtaatcagctgttaacattattataaccctacatgatggcatcttctagct

SEQ ID NO: 151 - RNA, 73 nt, synthetic construct
  source 1..73: mol_type = other RNA
  misc_feature 1..73: note = GEiGS solution sequence (pre-miRNA hairpin)
ggccggccatggcatgatcgtcttgatattgcttcctgtcacaatataaagaagatcatgtccatgaccgacc

SEQ ID NO: 152 - RNA, 92 nt, synthetic construct
  source 1..92: mol_type = other RNA
  misc_feature 1..92: note = GEiGS solution sequence (pre-miRNA hairpin)
gggcagtctttgctactttatgatttatttaacttgtggagtaaggtgttcagaggtccacaagttataaatcataaagcggcaggctgcca

SEQ ID NO: 153 - RNA, 72 nt, synthetic construct
  source 1..72: mol_type = other RNA
  misc_feature 1..72: note = GEiGS solution sequence (pre-miRNA hairpin)
ctcccctccaaaatgtattgttataacaacccggagtcgggttattataaccctacattttgtaaaaggatg

SEQ ID NO: 154 - RNA, 94 nt, synthetic construct
  source 1..94: mol_type = other RNA
  misc_feature 1..94: note = GEiGS solution sequence (pre-miRNA hairpin)
tgttttgagcgggggtaacattattataaccctacattttgtcataaacatgtagggatataataatcttacctcctctcatttgctatattca

SEQ ID NO: 155 - RNA, 72 nt, synthetic construct
  source 1..72: mol_type = other RNA
  misc_feature 1..72: note = GEiGS solution sequence (pre-miRNA hairpin)
ggagcttattataaccctacattttgttttataatttcaaaaaaccaaatatatgtggttataatttgcttc

SEQ ID NO: 156 - RNA, 94 nt, synthetic construct
  source 1..94: mol_type = other RNA
  misc_feature 1..94: note = GEiGS solution sequence (pre-miRNA hairpin)
ataaaggaagttaggatagttaagaggcagaagtatatttctattttccaaataacttatgcacgcttaactatccttgcttcctaacccgcgc

SEQ ID NO: 157 - RNA, 87 nt, synthetic construct
  source 1..87: mol_type = other RNA
  misc_feature 1..87: note = GEiGS solution sequence (pre-miRNA hairpin)
tctcagcctgtgacgtagggttataaaaatgttactgttgtctgaaagaaaagtaacattattataaccctacattacagtttgaga

SEQ ID NO: 158 - RNA, 87 nt, synthetic construct
  source 1..87: mol_type = other RNA
  misc_feature 1..87: note = GEiGS solution sequence (pre-miRNA hairpin)
tctcaggctgtgcacaagttaaataaatcataatctgtggtcagaaagaaaagtttatgatttatttaacttgtgtaccgtttggga

SEQ ID NO: 159 - RNA, 98 nt, synthetic construct
  source 1..98: mol_type = other RNA
  misc_feature 1..98: note = GEiGS solution sequence (pre-miRNA hairpin)
acgaatggctatgcactattggaaatttggagagtataatgaacacatagactataattgttcattataacaaatttccaatagtgcacaacctacac

SEQ ID NO: 160 - RNA, 96 nt, synthetic construct
  source 1..96: mol_type = other RNA
  misc_feature 1..96: note = GEiGS solution sequence (pre-miRNA hairpin)
cgggccccgggcgggcgacaaggataaataacatcataaaattgttttttcccccgccaattttatgatttatttaacttgtggcccccccggccc

SEQ ID NO: 161-166 - skipped (6 entries)

SEQ ID NO: 167 - RNA, 69 nt, synthetic construct
  source 1..69: mol_type = other RNA
  misc_feature 1..69: note = GEiGS solution sequence (pre-miRNA hairpin)
ccactagtaaagcgtgcgagagttaactttgaaactaaagaagttaacttatgcacgcttaactattgg

SEQ ID NO: 168 - RNA, 69 nt, synthetic construct
  source 1..69: mol_type = other RNA
  misc_feature 1..69: note = GEiGS solution sequence (pre-miRNA hairpin)
ccaccaggtttattggaattttgttactttgaaactaaagaagtaacaaatttccaataatcctgttgg

SEQ ID NO: 169 - RNA, 73 nt, synthetic construct
  source 1..73: mol_type = other RNA
  misc_feature 1..73: note = GEiGS solution sequence (pre-miRNA hairpin)
gctcacttcaagtatagcgtgcataagttatctgtgactttaaaagttaacttatgcacgcttaactatgagt

SEQ ID NO: 170 - RNA, 73 nt, synthetic construct
  source 1..73: mol_type = other RNA
  misc_feature 1..73: note = GEiGS solution sequence (pre-miRNA hairpin)
gctcccttcaaggtatattggaaatttgtttctgtgactttaaaagtaacaaatttccaataatcctgtgagt

SEQ ID NO: 171 - RNA, 68 nt, synthetic construct
  source 1..68: mol_type = other RNA
  misc_feature 1..68: note = GEiGS solution sequence (pre-miRNA hairpin)
cctatagtctggcgtgcataagtcaactgtgtgaaacaaaagttaacttatgcacgcttaactatagg

SEQ ID NO: 172 - RNA, 68 nt, synthetic construct
  source 1..68: mol_type = other RNA
  misc_feature 1..68: note = GEiGS solution sequence (pre-miRNA hairpin)
cctacaggtctattggaaatttgctactgtgtgaaacaaaagtaacaaatttccaataatcctgtagg

SEQ ID NO: 173 - RNA, 69 nt, synthetic construct
  source 1..69: mol_type = other RNA
  misc_feature 1..69: note = GEiGS solution sequence (pre-miRNA hairpin)
ccacactgttctttctatgtcttgtactttgaaactaaagaagtacaagagatagaaagaccagtctgg

SEQ ID NO: 174 - RNA, 73 nt, synthetic construct
  source 1..73: mol_type = other RNA
  misc_feature 1..73: note = GEiGS solution sequence (pre-miRNA hairpin)
gctcgcttccctgtactttctatctcttgttctgtgactttaaaagtacaagagatagaaagaccagtcgagt

SEQ ID NO: 175 - RNA, 68 nt, synthetic construct
  source 1..68: mol_type = other RNA
  misc_feature 1..68: note = GEiGS solution sequence (pre-miRNA hairpin)
cctgactgtgctttctatctcttctactgtgtgaaacaaaagtacaagagatagaaagaccagtcagg

SEQ ID NO: 176 - RNA, 64 nt, synthetic construct
  source 1..64: mol_type = other RNA
  misc_feature 1..64: note = GEiGS solution sequence (pre-miRNA hairpin)
cccagttcaagcgtctttacgttaaccggacctgagcgtttaacttatgcacgcttaactaggc

SEQ ID NO: 177 - RNA, 64 nt, synthetic construct
  source 1..64: mol_type = other RNA
  misc_feature 1..64: note = GEiGS solution sequence (pre-miRNA hairpin)
ccaaggacttattgcctatctgttaccggacctgagcgttaacaaatttccaataatcctgggc

SEQ ID NO: 178 - RNA, 64 nt, synthetic construct
  source 1..64: mol_type = other RNA
  misc_feature 1..64: note = GEiGS solution sequence (pre-miRNA hairpin)
cctctggatctttcccactattgtaccggacctgagcgttacaagagatagaaagaccagtggc

SEQ ID NO: 179 - RNA, 83 nt, synthetic construct
  source 1..83: mol_type = other RNA
  misc_feature 1..83: note = GEiGS solution sequence (pre-miRNA hairpin)
cggggttgactttccattctctgctggtcagggcagtgatgttgcccctcggcaaatccagcagagaatggaaagtcaccctg

SEQ ID NO: 180 - RNA, 84 nt, synthetic construct
  source 1..84: mol_type = other RNA
  misc_feature 1..84: note = GEiGS solution sequence (pre-miRNA hairpin)
aggcttgactttccattctctgctggtgagggtctaggataccacccggtaccggagatccagcagagaatcgaaagtcagcca

SEQ ID NO: 181 - RNA, 119 nt, synthetic construct
  source 1..119: mol_type = other RNA
  misc_feature 1..119: note = GEiGS solution sequence (pre-miRNA hairpin)
aggattctgctcatgccagggttgactttccattctctgctgggtggggtagggatattaggccccaattagaagatccatcagagaatggaaagtctccctggtgtgtggcatattca

SEQ ID NO: 182 - RNA, 119 nt, synthetic construct
  source 1..119: mol_type = other RNA
  misc_feature 1..119: note = GEiGS solution sequence (pre-miRNA hairpin)
aggattctgctcatgccagggggcgtctcggtggcacgacttggtggggtagggatattaggccccaattagaagaccaattcgtgccaccgagatgaccctggtgtgtggcatattca

SEQ ID NO: 183 - RNA, 83 nt, synthetic construct
  source 1..83: mol_type = other RNA
  misc_feature 1..83: note = GEiGS solution sequence (pre-miRNA hairpin)
cggggtaacaaatttccaataatcctgtcagggcagtgatgttgcccctcggaagctcaggattattggaaatttgttccctg

SEQ ID NO: 184 - RNA, 84 nt, synthetic construct
  source 1..84: mol_type = other RNA
  misc_feature 1..84: note = GEiGS solution sequence (pre-miRNA hairpin)
aggctaacaaatttccaataatcctgtgagggtctatgataccacccggtacaggagatcaggattattggcaatttgttgcca

SEQ ID NO: 185 - RNA, 119 nt, synthetic construct
  source 1..119: mol_type = other RNA
  misc_feature 1..119: note = GEiGS solution sequence (pre-miRNA hairpin)
aggattctgctcatgccagggtaacaaatttccaataatcctggtggggtagggatattaggccccaattagaagatcagtattattggaaatttgtcccctggtgtgtggcatattca

SEQ ID NO: 186 - RNA, 83 nt, synthetic construct
  source 1..83: mol_type = other RNA
  misc_feature 1..83: note = GEiGS solution sequence (pre-miRNA hairpin)
cggggttaacttatgcacgcttaactatcagggcagtgatgttgccccacggaagattagttaagcgtgcataagttaccctg

SEQ ID NO: 187 - RNA, 84 nt, synthetic construct
  source 1..84: mol_type = other RNA
  misc_feature 1..84: note = GEiGS solution sequence (pre-miRNA hairpin)
aggcttaacttatgcacgcttaactatgagggtctatgataccacccggtacaggagattagttaagcgtgattaagttagcca

SEQ ID NO: 188 - RNA, 119 nt, synthetic construct
  source 1..119: mol_type = other RNA
  misc_feature 1..119: note = GEiGS solution sequence (pre-miRNA hairpin)
aggattctgctcatgccagggttaacttatgcacgcttaactagtggggtagggatattaggccccaattagaagcttagctaagcgtgcataagtttccctggtgtgtggcatattca

SEQ ID NO: 189 - RNA, 83 nt, synthetic construct
  source 1..83: mol_type = other RNA
  misc_feature 1..83: note = GEiGS solution sequence (pre-miRNA hairpin)
cggggtacaagagatagaaagaccagttcagggcagtgatgttgcccctcggaagatactggtctttctatctcttgtccctg

SEQ ID NO: 190 - RNA, 84 nt, synthetic construct
  source 1..84: mol_type = other RNA
  misc_feature 1..84: note = GEiGS solution sequence (pre-miRNA hairpin)
aggctacaagagatagaaagaccagttgagggtctatgataccacccggtacaggagatactggtctttctctctcttgtgcca

SEQ ID NO: 191 - RNA, 119 nt, synthetic construct
  source 1..119: mol_type = other RNA
  misc_feature 1..119: note = GEiGS solution sequence (pre-miRNA hairpin)
aggattctgctcatgccagggtacaagagatagaaagaccagtgtggggtagggatattaggccccaattagaagatacttgtctttctatctcttgcccctggtgtgtggcatattca

SEQ ID NO: 192 - RNA, 21 nt, synthetic construct
  source 1..21: mol_type = other RNA
  misc_feature 1..21: note = gRNA sequence
ggattccgttgcccgctgtcc

SEQ ID NO: 193 - DNA, 760 bp, synthetic construct
  source 1..760: mol_type = other DNA
  misc_feature 1..760: note = HDR template sequence
cctcagtcaaactgaggacttcagatactctcatccacgaagacggttggggtggtttttccccaggaagtaagagggctatctttagcgagatgcgggctgaggcagccctgggggagtcactaccattgcagccctacccagaggtcctgggtccaccctccctcgccaatcctccccagggttccagagatggccaccagcctcggctctcccacgtctacacccatcacctccagcctgagggcgcaactgagaggaactgagacccaagcagctcagtaaagtatgtctgggggtcaggggcaccccatctgggccagggcgctgctcaggcaggagagcaggggcagtgtagtcagcagatagagaatgctctggagacaatctatctcttgtactacactgaacccgctgtccagccgttggcggggggcgggggagctcctgcccccaggtcactcgccctggggccacatcagaacccgcccatccccagtcccaggaggaagacccgggagtaggttgtgaaacggagtctagagggaacaagacgctcacctgggccacagaacaatagaaggggaggtgggggcctcccctgggaccgggtcccgatccggattagatccgtggggcaggattagccctgttggtcccacgaagcctgggccactggtgcggtgacggtggtggggcgggcttcggaatccgtcaggagggaagcgcgcccgtggggccggagaggaagtaaacccgc

(19) United States
(12) Patent Application Publication
(10) Pub. No.: US 2026/0218199 A1
(43) Pub. Date: Jul. 30, 2026

(54) NOVEL POLYNUCLEOTIDES, CELLS AND METHODS

(71) Applicant: Laverock Therapeutics Limited, London (GB)

(72) Inventors: Timothy Allsopp, London (GB); Vlad Seitan, London (GB); Vishal Menon, London (GB); Anna Klucnika, London (GB)

(73) Assignee: Laverock Therapeutics Limited, London (GB)

(21) Appl. No.: 19/146,760

(22) PCT Filed: Jan. 15, 2024
(86) PCT No.: PCT/EP2024/050828

§ 371 Date: Jul. 9, 2025

(30) Foreign Application Priority Data
Jan. 13, 2023 (GB) 2300553.1
May 12, 2023 (GB) 2307090.7
Jun. 19, 2023 (GB) 2309214.1

(51) Int. Cl. C12N 15/113; C12N 9/22; C12N 15/85; C12N 15/86; C12N 15/90

(52) U.S. Cl. CPC C12N 15/1138; C12N 9/226; C12N 15/85; C12N 15/86; C12N 15/907; C12N 2310/141; C12N 2740/15043

(57) ABSTRACT

There are disclosed inter alia novel inhibitory RNAs, or a polynucleotide sequence encoding an inhibitory RNA, and expression cassettes, vectors, virions, synthetic microRNA, synthetic pre-microRNA molecules and cells, and associated medical uses and methods of modifying protein expression, especially B2M protein expression.

### FIELD OF THE INVENTION

The present invention relates to novel inhibitory RNAs or polynucleotide sequences encoding inhibitory RNAs and expression cassettes, vectors, virions, synthetic microRNAs and synthetic pre-microRNAs, cells comprising the inhibitory RNAs or polynucleotide sequences encoding inhibitory RNAs and compositions comprising the cells. The present invention also relates to novel inhibitory RNAs or polynucleotide sequences encoding inhibitory RNAs, expression cassettes, vectors, virions, synthetic microRNA and synthetic pre-microRNAs and cells for use in therapy and in methods of treating, for example, autoimmune diseases or cancer, as well as inter alia related methods of degrading the messenger RNA transcribed, for example from the beta-2 microglobulin (B2M) gene in a cell. The present invention further relates inter alia to methods of modifying the expression of a protein or translation of a messenger RNA in a cell.

### BACKGROUND

Cellular therapies are a class of advanced medical therapies in which patients are treated through the transplantation of cells that may or may not have undergone prior engineering to enhance their functionality. Cellular therapies such as bone marrow transplantation or blood transfusions are examples of well-known cellular therapy that have existed in medicine for many decades. However, more recently, through the use of genetic engineering techniques, novel cell therapies have been developed and used successfully in many different disease indications such as diabetes, cancer and blindness.

Cell therapies can be autologous, that being therapeutic cells that originate from the patient that is being treated or allogeneic where the cells originate from an unrelated donor. Autologous approaches have the benefit of maximising the likelihood of engraftment post-transplantation but can be expensive to manufacture and may not be applicable to very sick patients that are not capable of donating their cells for preparation or who suffer from terminal illnesses.

In contrast, allogeneic cell therapies can in principle be manufactured more efficiently from healthy donors, reducing cost, and in theory can be administered off-the-shelf and be used to treat acutely ill, recently diagnosed patients. However, allogeneic therapies currently require aggressive immunosuppression to reduce the risk of graft rejection post-transplantation. Even then, administration of immunosuppression does not guarantee the rejection of the graft over a longer period. To become mainstream, allogeneic cell therapies will have to overcome the graft rejection barrier.

Graft rejection is primarily driven by the host adaptive immune system reacting to unmatched Major Histocompatibility Complex I (MHC-I complex) expressed on the surface of transplanted cells. The MHC proteins, also known as human leukocyte antigens, are encoded by a cluster of gene complexes known as the human leukocyte antigen (HLA) system. HLA genes are co-dominantly expressed and are highly polymorphic. The expression of many different alleles is advantageous for the adaptive immune system to defend against cancerous cells or pathogens such as viruses or bacteria. However, it is this high degree of polymorphism that presents a major barrier to allogenic transplants such as allogenic cell therapies.

MHC-I is present on the surface of virtually all human cells and, as discussed, plays important roles in immunosurveillance (FIG. 1) and in recognition of self from non-self. MHC-I is a dimer of β2-microglobulin and an HLA chain. The HLA chain is highly polymorphic, i.e., individuals express very diverse combinations of the dimer in the population. Consequently, the immune system is trained to selectively tolerate only those MHC-I variants expressed by the individual. In the context of a transplant, unless the donor and host express exactly the same HLA variants, which is highly unlikely unless close blood relatives are involved, the host immune system will recognise the non-self MHC-I variants on the surface of the donor cells and will mount a response (also known as alloimmunity). MHC-I is therefore the main source of immunogenicity in allogeneic transplants.

Numerous approaches have been taken to overcome the graft rejection barrier. Traditionally, graft recipients receive life-long immunosuppression, which is not only not always effective but also leaves the recipient vulnerable to serious illness such as infectious disease or cancer. Genetic engineering approaches have also been considered. One such approach is to generate hypoimmunogenic cells by completely knocking out (KO) MHC-I expression (see, e.g., WO2012/145384). While this strategy successfully evades reaction from the host adaptive immunity, it exposes the graft to rejection by cells of the innate immune system through the activity of Natural Killer (NK) cells. NK cells play an important role in detection of aberrant cells and react strongly against cells that lack MHC-I expression, as this is sometimes a hallmark of malignant cells. The complete absence of MHC-I is also referred to as the “missing self” phenotype, and these cells are effectively detected and lysed by NK cells. At the mechanistic level, this is triggered because MHC-I molecules function as inhibitory ligands for NK cells. MHC-I KO cells need additional engineering to stably express tolerising ligands to avoid killing by innate immune cells (FIG. 2).

The present invention aims to address this problem by creating hypoimmunogenic cells through partial silencing of MHC-I. As discussed, the MHC-I complex is a heterodimer made up of a hypervariable HLA protein and beta-2 microglobulin (B2M). By partially knocking down B2M expression, there is partial silencing of MHC-I which can allow transplanted cells to evade both the innate (NK-mediated) and adaptive (T-cell mediated) immune system driven rejection.

One approach to generate hypoimmunogenic cells is to use inhibitory RNAs, e.g. via the Gene Editing induced Gene Silencing (GEiGS®) system, to modulate the expression of MHC-I down to a level that prevents a significant adaptive immune response, while still being sufficient to inhibit the NK cell mediated response. GEiGS is a gene silencing technology that functions by redirecting the silencing specificity of endogenous non-coding RNAs (ncRNAs) with RNAi-mediated silencing activity towards a desired target sequence. In humans, the redirected ncRNAs are often microRNAs (miRNAs). These home in on their targets through sequence complementarity, therefore by changing their sequence it is possible to shift their silencing specificity towards a desired target of choice. Precise gene edits are made to the sequence of endogenous miRNAs, in order to provide them with the ability to engage a new desired target through perfect sequence complementarity and silence it via the RNAi pathway.

GEiGS therefore works by hijacking silencing non-coding RNAs, such as miRNAs, that are already expressed in the cell and redirecting them towards silencing/modulating the expression of a desired target. The GEiGS target is silenced through the RNAi pathway. However, unlike siRNA or shRNA in which silencing is induced through administration of double stranded oligos or transgenes, respectively, GEiGS makes use of endogenous ncRNAs as vectors for silencing the desired targets.

Modifying the sequence of endogenous ncRNAs to redirect their specificity is achieved through genome editing technologies, and GEiGS is not dependent on any particular technology and as such can be used with CRISPR, TALENs, zinc finger nucleases and any of their derivatives. While the “engine” of GEiGS (how silencing of the target is achieved) is based on the RNAi mechanism/pathway, the implementation of the technology (how the endogenous ncRNA is hijacked to target the desired gene) is based on genome editing technologies. For these reasons, GEiGS draws strengths from both RNAi and genome editing, enabling stable, tuneable, programmable and specific gene silencing.

GEiGS is particularly well-suited to partial silencing of MHC-I as it can deliver stable and tuneable silencing across a wide range of target expression. By employing GEiGS to modulate the expression of B2M it is possible to achieve the optimum level of MHC-I expression for evading both T cell- and NK cell-mediated responses (FIG. 3). Previous studies have verified the validity of some aspects of this approach in a research setting using oligo-based and transgenic RNAi (Karabekian et al., 2015—Tissue Eng Part A. 2015 October; 21(19-20):2559-71. doi: 10.1089/ten.TEA.2015.0105), but its implementation in human cells for therapeutics has been prevented by the limitations of existing RNAi technologies. GEiGS overcomes some of these limitations, paving the way for this approach to generate hypoimmunogenic cells to be applied in a clinical setting.

Other approaches to using inhibitory RNAs to partially or completely silence gene expression are also known in the art, e.g. by inserting a suitable expression construct into the genome of a cell to drive expression of an inhibitory RNA, by providing a suitable expression construct into the cell to drive expression of an inhibitory RNA episomally (e.g. on a suitably plasmid or other vector), or by direct delivery of the inhibitory RNA to a cell.

The potential of partial MHC-I silencing was exemplified in an in vivo study in which hypoimmunogenic islet cells were generated by siRNA-mediated MHC-I knock-down and subsequently transplanted in NOD mice, showing improved graft survival (Wang et al., 2012). While this study shows the efficiency of RNAi-mediated gene silencing to partially silence MHC-I, the transient nature of siRNA means the approach would not be feasible for a clinical application. On the other hand, GEiGS for example, would be perfectly suitable for this approach because it has the potential to provide stable and tuneable silencing.

Further advantages of the GEiGS approach include the simplicity in generating hypoimmunogenic cells, whereby the editing is of a non-coding gene and requires no additional transgene expression.

The generation of hypoimmunogenic cells is not solely reliant on the GEiGS approach and other approaches to generate these cells may be used. Other such alternative approaches include direct insertion of DNA into the genome using techniques such as homology directed repair and episomal methods such as transduction techniques that use viral vectors to deliver the DNA or RNA sequences of interest into the cell.

Overall, there remains a need for a producing off-the-shelf cellular therapies that overcome the significant and complex challenge of graft rejection. It is an object of the present invention to provide a novel approach of producing hypoimmunogenic cells that may be suitable for cellular therapies due to their ability to successfully evade both innate and adaptive immunity. Such cells may be produced using GEiGS technology or alternative approaches to express inhibitory RNAs to reduce B2M expression in the cells.

### SUMMARY OF THE INVENTION

The invention is based, in part, on the surprising finding that the expression of beta-2 microglobulin (B2M) can be modified using inhibitory RNAs, e.g. provided in the genome or episomally (such as by GEiGS or vector transduction), to allow for a tuneable expression of cell surface B2M to permit cells to evade the innate and adaptive immune systems. This has the particular advantage of being able to facilitate the generation of “off-the-shelf” allogenic transplantable cells that are less susceptible to rejection by the recipient host immune system.

According to a first aspect of the invention, there is provided herein an inhibitory RNA, or a polynucleotide sequence encoding an inhibitory RNA, preferably a microRNA, the inhibitory RNA comprising or consisting of a sequence selected from:
(i) SEQ ID NOs: 1-17, 64-127;
(ii) a variant of SEQ ID Nos: 1-17, 64-127;
(iii) a fragment of SEQ ID Nos: 1-17, 64-127; and
(iv) a DNA sequence complementary to any one of (i), (ii) or (iii).

According to a second aspect of the invention, there is provided an expression cassette comprising a polynucleotide sequence encoding an inhibitory RNA according to the invention operably linked to one or more regulatory elements suitable for permitting transcription of the inhibitory RNA in a cell, preferably a promoter, optionally a constitutive promoter or an inducible promoter.

According to a third aspect of the invention, there is provided a vector comprising a polynucleotide sequence encoding an inhibitory RNA according to the invention or an expression cassette according to the invention.

According to a fourth aspect of the invention, there is provided a virion comprising the vector of the invention.

According to a fifth aspect of the invention, there is provided a synthetic microRNA comprising or consisting of a sequence selected from any one of SEQ ID NOs: 1-17, 64-127 or functional variants or fragments thereof.

According to a sixth aspect of the invention, there is provided a synthetic pre-microRNA comprising or consisting of a sequence selected from any one of SEQ ID NOs: 18-34, 128-191 or a functional variant or fragment thereof.

According to a seventh aspect of the invention, there is provided a synthetic microRNA adapted to target a messenger RNA transcribed from the B2M gene, the synthetic microRNA comprising a sequence according to any one of SEQ ID NOs: 1-17, 64-127 or a functional variant or fragment thereof, optionally wherein the synthetic microRNA comprises a sequence according to any one of SEQ ID NOs: 18-34, 128-191 or a functional variant or fragment thereof.

According to an eighth aspect of the invention, there is provided a cell comprising or expressing the synthetic microRNA according to the invention, wherein the cell is modified to express the synthetic microRNA such that the synthetic microRNA targets a messenger RNA transcribed from the B2M gene.

According to a ninth aspect of the invention, there is provided a cell comprising or expressing a sequence encoding a synthetic inhibitory RNA, preferably a synthetic microRNA, wherein the synthetic inhibitory RNA is adapted to target B2M.

According to a further aspect of the invention, there is provided a cell comprising the expression cassette, vector, virion, synthetic microRNA or pre-microRNA of the invention.

According to a further aspect of the invention, there is provided a composition comprising a cell of the invention.

In a further aspect of the invention, there is provided an inhibitory RNA or polynucleotide encoding an inhibitory RNA, an expression cassette, a vector, a virion, a synthetic microRNA or pre-microRNA, a cell or a composition of the invention for use in therapy.

In a further aspect of the invention, there is provided herein an inhibitory RNA or polynucleotide encoding an inhibitory RNA, an expression cassette, a vector, a virion, a synthetic microRNA or pre-microRNA, a cell or a composition of the invention for use in a method of treating an autoimmune disease or cancer in a subject, wherein the autoimmune disease is preferably Type I diabetes.

In a further aspect of the invention, there is provided herein an inhibitory RNA or polynucleotide encoding an inhibitory RNA, an expression cassette, a vector, a virion or a synthetic microRNA or pre-microRNA of the invention for use in a method of degrading the messenger RNA transcribed from the B2M gene in a cell, preferably wherein the messenger RNA is partially silenced and B2M protein expression is modified in a range of 1%-90%, optionally 5%-90%, optionally 10%-90%, of expression compared to a control cell. Various preferred levels of expression are discussed herein.

In yet another aspect of the invention, there is provided an inhibitory RNA or polynucleotide encoding an inhibitory RNA, an expression cassette, a vector, a virion or a synthetic microRNA or pre-microRNA of the invention for use in a method of degrading the messenger RNA transcribed from the B2M gene in a cell, preferably wherein the messenger RNA is partially silenced and B2M protein expression is modified in a range of 1%-90%, optionally 5%-90%, optionally 10%-90%, of expression compared to a control cell.

In some embodiments, the expression level of B2M in a cell according to the present invention is, for example, within a range of 1%-80%, 1%-70%, 1%-60%, 1%-50%, 1%-40%, 1%-30%, 1%-20%, 1%-10%, 1%-5%, 5%-80%, 5%-70%, 5%-60%, 5%-50%, 5%-40%, 5%-30%, 5%-20%, 5%-10%, 10%-80%, 10%-70%, 10%-60%, 10%-50%, 10%-40%, 10%-30%, 10%-20%, 20%-90%, 30%-90%, 40%-90%, 50%-90%, 10%-20%, 20%-30%, 30%-40%, 40%-50%, 50%-60%, 60%-70%, 70%-80%, or 80%-90% when compared to a control cell. In some embodiments the expression level of B2M in a cell is determined by the amount of B2M expressed on the surface of cell. The amount of B2M present on the surface of cell can be determined by various conventional techniques, e.g. by flow cytometry.

In a further aspect of the invention, there is provided a cell of the invention or a composition of the invention for use in a method of treating an autoimmune disease or cancer in a subject, wherein the method comprises administering the cell or composition in a therapeutic amount to the subject.

In a further aspect of the invention, there is provided a method of treating an autoimmune disease or cancer in a subject, preferably wherein the autoimmune disease is Type I diabetes, wherein the method comprises administering the cell or composition in a therapeutic amount to the subject.

In a further aspect of the invention, there is provided a use of a cell of the invention or a composition of the invention in the manufacture of a medicament for treating an autoimmune disease or cancer in a subject, preferably wherein the autoimmune disease is Type I diabetes.

In a further aspect of the invention, there is provided a method of modifying the expression of a protein in a cell comprising introducing into a cell an inhibitory RNA or polynucleotide encoding an inhibitory RNA, an expression cassette, a vector, a virion or a synthetic microRNA or pre-microRNA of the invention.

In a further aspect of the invention, there is provided a method of modifying the translation of a messenger RNA transcript derived from a protein coding gene and subsequent expression of a protein in a cell comprising introducing into the cell an inhibitory RNA or polynucleotide encoding an inhibitory RNA, an expression cassette, a vector, a virion or a synthetic microRNA or pre-microRNA of the invention.

In a further aspect of the invention, there is provided a method of modifying the translation of an messenger RNA transcript derived from a protein coding gene and subsequent expression of a protein in a cell comprising introducing into the cell a DNA editing agent conferring a silencing specificity of a microRNA towards a target RNA of interest, wherein the microRNA is modified such that the sequence of the microRNA comprises any one of SEQ ID NOs: 1-17, 64-127 thereby modifying the translation of the messenger RNA transcript into a protein molecule.

In a further aspect of the invention, there is provided a cell or cell population obtained from the method of the invention.

In a further aspect of the invention, there is provided a composition comprising the cell or cell population of the invention.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES AND SEQUENCES

Embodiments of the invention are further described hereinafter with reference to the accompanying drawings, in which:

FIG. 1 shows a schematic representation MHC-I on the surface of cells and the immune surveillance system.

FIG. 2 shows a schematic illustration of the interaction of cells comprising non-self MHC-I and those completely lacking MHC-I with cells of the adaptive and innate immune system.

FIG. 3 illustrates a strategy to generate hypoimmune cells based on downregulating MHC-I expression to an optimum level that enables evading both T cell- and NK cell-mediated responses.

FIG. 4 shows a schematic illustration of the use of the GEiGS workflow to develop, test and implement modified cells according to the present invention.

FIG. 5 shows a plasmid map of plasmid VB210602-1567ytv that was used for ectopic (episomal) expression to assess the ability of miRNA sequences determined by GEiGS (“GEiGS solutions”) to silence B2M expression.

FIG. 6 shows a graph of small RNA real-time PCR (RT-PCR) in iPSCs transfected with plasmids expressing either a negative control-expressing plasmid, or B2M-targeting Solution 43. The GEiGS solution is only detected in the corresponding sample indicating the specificity of the assay. Solution 43 is expressed at comparable levels to a panel of endogenous miRNAs.

FIG. 7 shows flow cytometry method and gating to assess the ability of a given GEiGS silencing RNA to reduce B2M expression.

FIG. 8 shows a graph illustrating the ability or a range of inhibitory/silencing RNA sequences developed through the GEiGS process (“GEiGS solutions”) to reduce B2M expression in cells as determined by flow cytometry. Each GEiGS solution is a combination of miRNA scaffold and specific sequence changes complementary to the B2M mRNA that trigger its degradation. Solutions are developed via the GEiGS computational workflow to reduce B2M expression Series 1 illustrates the result for all cells expressing DsRed (all successfully transfected cells), while series 2 shows the results for the top 20%, i.e. cells expressing high levels of DsRed and therefore high levels of the GEiGS solution (silencing RNA).

FIGS. 9 and 10 show mean silencing activity of GEiGS in clonal cell lines in which GEiGS Solutions are knocked-in to either one or both alleles corresponding to the genomic location of the original encoded miRNA, using gene editing techniques. Data is expressed as residual B2M expression measured by flow cytometry, averaged over multiple clones of the same genotype.

FIG. 11 shows a) results indicating a reduction in surface B2M expression. Of the CD3 positive, mixed T cell population approximately 24% of the cells demonstrated a silencing of B2M, with a reduction in mean fluorescent intensity of 80%, compared with control unedited cells; b) Quantification of B2M silencing in helper (CD4+) and cytotoxic (CD8+) T cells, showing efficient silencing of B2M in both compartments (by ~90%).

FIG. 12 shows that partial knockdown of B2M results in reduced specific lysis by activated NK cells in tissue culture (corresponding to enhanced cell survival following exposure to the innate (NK-mediated) immune system). NK and target cells were cultured under different experimental conditions including exposure to NK-cell activating cytokines IL-2 and/or IL-12. Control iPSC displayed a reduced level of specific lysis compared to a positive control K562 cell line, known to be a potent stimulator of activated NK cell mediated killing. Undifferentiated B2M-iPSC showed elevated levels of specific lysis as complete absence of B2M results in complete absence of MHC-I (“missing self” phenotype), detection and lysis by activated NK cells. Undifferentiated, GEiGS-modified iPSC lines (iPSC^{S29/29}) displayed consistently reduced specific lysis by NK cells compared the B2M^{-/-} cell lines across all mixed culture conditions tested.

FIG. 13 shows miRNA expression of the endogenous miRNAs of solutions 12 and 30 in iPSCs and pancreatic progenitor cells. It can be seen that expression of the solution 30 miRNA (hsa-mir-21) is specific to pancreatic progenitor cells, whereas the solution 12 miRNA (hsa-mir-302c) is specific to iPSCs.

FIG. 14 shows the ability of a Solution 30 heterozygous IPSC line to differentiate into CXCR4 expressing definitive endoderm cells. The heterozygous knock-in line (iPSC miR-21^{S30/+1}) showed a high proportion of CXCR4 positive cells (approximately 97%), compared with both control (unmodified, 50%) and B2M-iPSCs (95%)

FIG. 15A shows reduced B2M expression in CXCR4 expressing cells generated via in vitro differentiation of the Solution 30 heterozygous IPSC line. The iPSC miR-21^{S30/+1} line showed an approximately 80% reduction in B2M expression compared with control (unmodified) iPSC cell line. As predicted B2M^{-/-} iPSCs do not express the B2M protein.

FIG. 15B shows expression of pancreatic and duodenal homeobox-1 (PDX-1) and B2M in pancreatic progenitor cells. Results indicate that a significant proportion of the specified progenitors are positive for PDX-1, a marker gene which is necessary for pancreatic development and Beta-cell maturation. The majority of PDX-1 positive cells also stained for B2M. The iPSC miR-21^{S30/+1} clonal line showed a high proportion of PDX-1 expressing cells which had markedly reduced B2M expression compared with the control (unmodified) iPSCs. FIG. 16a. Efficient generation of monocytes from iPSC. The iPSC lines (GEiGS Solution 30 (S30/S30 genotype) and isogenic control) were used to generate enriched monocytes using published protocols, from which greater than 90% of the cells were double positive for the known monocyte markers CD14 and CD45.

FIG. 16b. Context specific silencing of B2M in iPSC-derived monocytes. The iPSC lines (GEiGS Solution 30 and isogenic control) were used to generate monocytes which were then stained for residual cell surface B2M expression using flow cytometry (as described for FIGS. 9, 10 and 12). When expanded as undifferentiated iPSC, greater than 93% of the cells for both the isogenic control and GEiGS Solution 30 clonal lines expressed B2M, whereas following differentiation into monocytes the GEiGS Solution 30 line showed a marked reduction in the number of cells with the same quantity of cell surface expressed B2M compared with the isogenic control line (16% cells with residual B2M compared with 77%).

FIG. 17 shows effects of homology arm length on efficiency of homology dependent repair (HDR). Flanking genomic sequences (homology arms) of length 40 base pairs show superior effects for the efficiency of HDR (% HDR) and in a dose dependent manner, compared to flanking genomic sequences of length 350 bp or length 150-350 bp which are only marginally better to no donor template alone.

FIG. 18 shows Solution 29's ability to buffer the induction of B2M expression (as measured by MHC-I detection) in edited cells following their exposure to inflammatory stimuli (IFN-γ). FIGS. 19a and 19b show that multiple Solutions can silence their target genes when ectopically expressed from a single construct with a constitutive promoter in iPSC. The plasmid map for expression of one or more Solutions is shown in FIG. 19a. The plasmid construct is depicted with a miR-30 based Solution targeting eGFP downstream of dsRed reporter in the mutlple cloning site (MCS), driven by the constitutive EF1a promoter. The eGFP targeted Solution is replaced with one or more Solutions designed against B2M and eGFP or B2M alone (see Example 14). FIG. 19b shows that Solution 101, consisting of miRNA-30 repurposed to target GFP alone, demonstrated an efficient silencing of GFP without affecting the level of expression of B2M in the undifferentiated iPSC and that Solution 179, consisting of two distinct modified miRNA scaffolds (miRNA-20a and miRNA-30), was effective in silencing both B2M and eGFP with apparent equal efficiency.

FIG. 20 shows that Solution 177, consisting of modified miRNA-518b (Solution 43) and miRNA-20a (Solution 29), distinct Solutions with diverse sequences targeting the B2M transcript (triggers) appeared more effective in silencing B2M when compared to the Solution 43 alone, indicating a minor additive effect and that the two distinct Solutions do not interfere in each others' ability to silence B2M. Whereas Solution 178 which possesses two copies of Solution 29 and the same trigger sequences, does not have an additive effect in silencing B2M compared with Solution 29 alone.

FIGS. 21a, b and c show results of a study indication that GEiGS is very specific, showing no off-target effects. In particular a GEiGS approach as described herein to silence B2M was highly specific, showing no significant off-target effects on expression of other than B2M. The MA plots of the differential gene expression analysis show log 2 of mean count per million on the x-axis and log 2 of fold-change on the y-axis. Dots represent individual genes tested. Significantly downregulated genes are highlighted in blue, significantly up-regulated genes are highlighter in red). Genes with no significant change in expression level are shown in black. FIG. 21a shows transcriptomic data from cells modified by a CRISPR B2M knock out compared to the control WT/WT parental cell line. The significantly reduced expression level of B2M compared to control is highlighted. No other significant changes in expression levels are for other genes are evident. FIG. 21b shows transcriptomic data in cells in which the miRNA which hsa-mir-20a has been knocked out compared to the control WT/WT parental cell line. Note—hsa-mir-20a is the miRNA which is edited when ‘Solution 29’ is introduced via GEiGS. No significant changes in expression levels are evident as a result of the knock out of hsa-mir-20a. FIG. 21c shows transcriptomic data in cells in which the miRNA which Solution 29 has been introduced via GEiGS compared to the control WT/WT parental cell line. As can be seen, as with the CRISPR KO, only B2M shows a significant alteration in expression. No other significant changes in expression levels are for other genes are evident.

FIGS. 22a and b show that B2M silencing in myeloid cells results in loss of cell surface HLA-A/B/C (and therefore MHC-I) in monocytes and macrophages.

FIG. 23a shows constructs used to validate the use of lentiviral transduction and a pooled screening strategy. FIG. 23b shows use of lentiviral transduction and a pooled screening strategy to identify ‘hits’ from designed GEiGS Solutions using primary human T cells.

FIG. 24a shows validation of the lentiviral based pooled screening strategy in primary human T cells using verified Solutions for both eGFP and B2M.

FIG. 24b shows DsRed and B2M knockdown (GFP regression) corresponding to individual Solutions in combined solution lentiviruses in Jurkats.

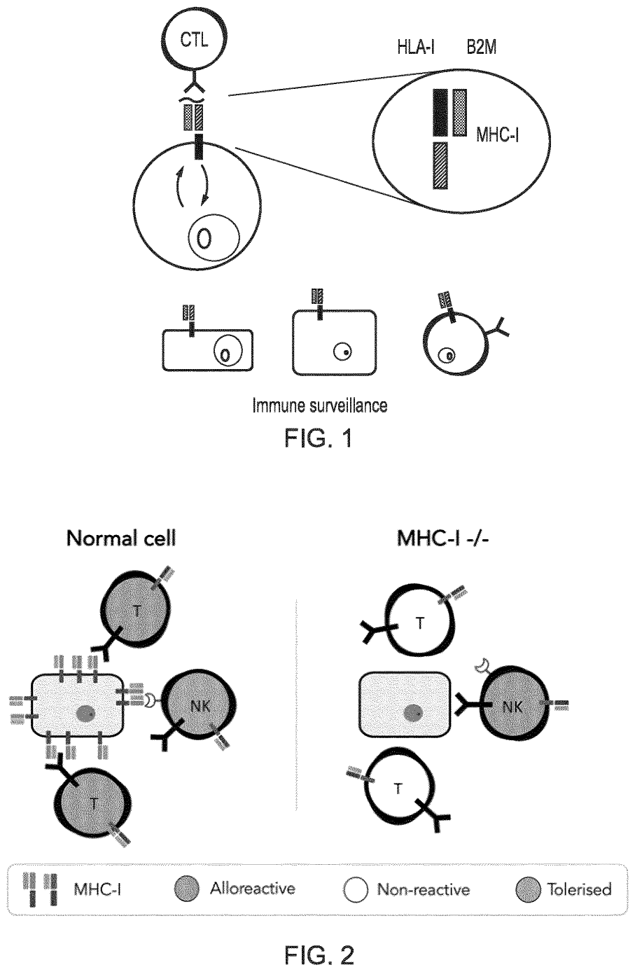

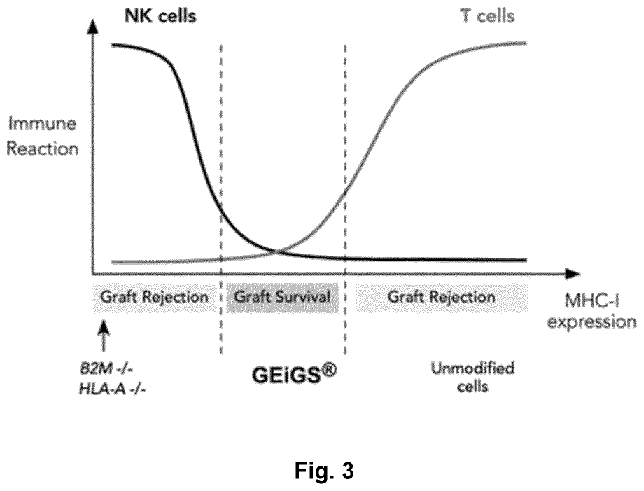

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION OF THE INVENTION

The present invention, in some embodiments thereof, relates to inhibitory RNAs, including microRNAs and, more particularly, but not exclusively, to the use of the same for degrading endogenous target RNA of interest in eukaryotic cells. The invention, in some aspect relates to the use of synthetic inhibitory RNAs in cells to reduce, but not eliminate, the expression of B2M to allow such cells to evade the innate and adaptive immune system of an animal (typically a human) into which the cells are introduced, e.g. for therapeutic purposes.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

All publications, patents and patent applications cited herein, whether supra or infra, are hereby incorporated by reference in their entirety.

### Definitions

The term “RNA interference” and the term “RNAi” are synonymous and refer to the process by which a polynucleotide (a miRNA or siRNA) comprising at least one polyribonucleotide unit exerts an effect on a biological process. The process includes, but is not limited to, gene silencing by degrading mRNA, attenuating translation, interactions with tRNA, rRNA, hnRNA, cDNA and genomic DNA, as well as methylation of DNA with ancillary proteins. An “Inhibitory RNA” is an RNA molecule capable of RNA interference, either as is or after cellular processing. Thus, the term includes, inter alia, RNA precursors that are processed to form a miRNA, siRNA, or other RNA that can degrade target mRNA.

The term “gene silencing” refers to a process by which the expression of a specific gene product is lessened or attenuated by RNA interference. The level of gene silencing (also sometimes referred to as the degree of “knockdown” or reduction of expression) can be measured by a variety of means, including, but not limited to, measurement of transcript levels by Northern Blot Analysis, B-DNA techniques, transcription-sensitive reporter constructs, expression profiling (e.g. DNA chips), qRT-PCR and related technologies. Alternatively, the level of silencing can be measured by assessing the level of the protein encoded by a specific gene. This can be accomplished by performing a number of studies including antibody-based detection such as Western Analysis and flow cytometry, measuring the levels of expression of a reporter protein that has e.g. fluorescent properties (e.g., GFP) or enzymatic activity (e.g. alkaline phosphatases), or several other procedures. Typically the level of silencing or reduction is compared to the level of expression of a given protein or mRNA in a control cell, as appropriate.

The terms “microRNA”, “miRNA”, or “miR” are synonymous and all refer to non-coding RNAs of, for example, about 19-24 nucleotides in length (and also, as the context will indicate, to DNA sequences that encode such RNAs) that are capable of entering the RNAi pathway and regulating gene expression. “Primary miRNA” or “pri-miRNA” represents the non-coding transcript prior to Drosha processing and includes the stem-loop structure(s) as well as flanking 5′ and 3′ sequences. “Precursor miRNAs” or “pre-miRNA” represents the non-coding transcript after Drosha processing of the pri-miRNA. The term “mature miRNA” can refer to the double stranded product resulting from Dicer processing of pre-miRNA or the single stranded product that is introduced into RISC following Dicer processing. In some cases, only a single strand of an miRNA enters the RNAi pathway. In other cases, both strands of a miRNA are capable of entering the RNAi pathway. miRNAs are found in a wide range of organisms (e.g. insects, mammals, plants, nematodes) and have been shown to play a role in development, homeostasis, and disease aetiology.

The term “silencing RNA form” or “silencing RNAs” or “silencing RNA molecule”, “sRNA” or “trigger sequence/RNA” refers to the mature small RNA being capable of hybridizing with a target RNA (or fragment thereof) and engage the RNAi pathway.

The term “target RNA” refers to a specific RNA that is targeted by the RNAi pathway, resulting in a decrease in the functional activity of the RNA. In some cases, the RNA target is a mRNA (typically B2M) whose functional activity is its ability to be translated. In such cases, the RNAi pathway will decrease the functional activity of the mRNA by translational attenuation or by cleavage. In the instant disclosure, target RNAs are targeted by non-naturally occurring miRNAs. The term “target” can also refer to DNA.

The term “endogenous miRNA” refers to a miRNA produced in an organism through transcription of sequences that naturally are present in the genome of that organism. Endogenous miRNA can be localized in, for example introns, open reading frames (ORFs), 5′ or 3′ untranslated regions (UTRs), or intergenic regions. The organism which produces an endogenous miRNA may be, without limitation, human (and other primates), mouse, rat, fly, worms, fish or other organisms that have an intact RNAi pathway. In some embodiments of the present invention endogenous miRNA loci are altered to retarget the endogenous miRNA to a new target (see, e.g. WO2019/058253, WO2020/183414 and WO2020/183419 for some suitable approaches using the approach referred to as GEiGS).

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn; Align Sequence Nucleotide BLAST) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes, including the wobble base pair formed between U and G. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

Perfect complementarity or 100% complementarity refers to the situation in which each nucleotide unit of one polynucleotide strand can hydrogen bond with a nucleotide unit of a second polynucleotide strand. Partial complementarity refers to the situation in which some, but not all, nucleotide units of two strands can hydrogen bond with each other. For example, two strands are at least partially complementary when at least 6-7 base pairs can be formed over a stretch of about 19-25 nucleotides. Sequences are said to be “complementary” to one another when each sequence is the (partial or complete) reverse complement (RC) of the other. For example, the sequence 5′ GATC 3′ is perfectly complementary to its reverse complement sequence 3′ CTAG 5′. Sequences can also have wobble base pairing.

The term “expression cassette” as used herein includes a polynucleotide sequence encoding a polypeptide to be expressed and sequences controlling its expression such as a promoter and optionally an enhancer sequence, including any combination of cis-acting transcriptional control elements.

The term “eukaryotic cell” as used herein refers to any cell of a eukaryotic organism. Eukaryotic organisms include single- and multi-cellular organisms. Single cell eukaryotic organisms include, but are not limited to, yeast, protozoans, slime molds and algae. Multi cellular eukaryotic organisms include, but are not limited to, animals (e.g. mammals, insects, invertebrates, nematodes, birds, fish, reptiles and crustaceans), plants, fungi and algae (e.g. brown algae, red algae, green algae).

As used herein, the phrase “stem cells” refers to cells which are capable of self renewing while in an undifferentiated state for extended periods of time in culture, but also retain the capacity to undergo differentiation into one or more different cell types having a particular, specialized function (e.g., fully differentiated cells). Stem cells can be totipotent, pluripotent, multipotent or unipotent until induced to differentiate into other cell types. Totipotent cells, such as embryonic cells within the first couple of cell divisions after fertilization are the only cells that can differentiate into embryonic and extra-embryonic cells and are able to develop into a viable human being. Preferably, the phrase “pluripotent stem cells” refers to cells which can differentiate into all three definitive embryonic germ layers, i.e., ectoderm, endoderm and mesoderm or remaining in an undifferentiated state. The pluripotent stem cells include embryonic stem cells (ESCs) and induced pluripotent stem cells (iPSC). The multipotent stem cells include adult stem cells and hematopoietic stem cells.

“Induced pluripotent stem cells” (iPSC; embryonic-like stem cells) refers to cells obtained by de-differentiation of adult somatic cells to endow cells with pluripotent properties, i.e., being capable of differentiating into the three embryonic germ cell layers, i.e., endoderm, ectoderm and mesoderm. Such cells may be obtained from a differentiated tissue (e.g., a somatic tissue such as skin) and undergo de-differentiation by genetic manipulation which reprogram the cell to acquire embryonic stem cells characteristics. The induced pluripotent stem cells may be formed by inducing the expression of Oct-4, Sox2, Kfl4 and c-Myc in a somatic cell. Induced pluripotent stem cells (iPSC) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 such as described in Park et al. Reprogramming of human somatic cells to pluripotency with defined factors. Nature (2008) 451:141-146. iPSCs can be human or non-human.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after early gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a foetus any time during gestation, preferably before 10 weeks of gestation, and cells originating from an unfertilized ova which are stimulated by parthenogenesis (parthenotes).

The embryonic stem cells of some embodiments of the invention can be obtained using well-known cell-culture methods. For example, human embryonic stem cells can be isolated from human pre-implantation blastocysts. Human blastocysts are typically obtained from human in vivo preimplantation embryos or from in vitro fertilized (IVF) embryos. Alternatively, a single cell human embryo can be expanded to the blastocyst stage.

It will be appreciated that commercially available stem cells can also be used according to some embodiments of the invention. Human ES cells can be purchased from the NTH human embryonic stem cells registry [www.grants.nih.gov/stem_cells/registry/current.htm].

In addition, embryonic stem cells can be obtained from various species, including mouse (Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

According to one embodiment, the stem cells utilized by some embodiments of the invention are bone marrow (BM)-derived stem cells including hematopoietic, stromal or mesenchymal stem cells [Dominici, M et al., (2001) J. Biol. Regul. Homeost. Agents. 15: 28-37]. BM-derived stem cells may be obtained from iliac crest, femora, tibiae, spine, rib or other medullar spaces.

Hematopoietic stem cells (HSCs), which may also referred to as adult tissue stem cells, include stem cells obtained from blood or bone marrow tissue of an individual at any age or from cord blood of a newborn individual. Preferred stem cells according to this aspect of some embodiments of the invention are embryonic stem cells, preferably of a human or primate (e.g., monkey) origin.

Mesenchymal stem cells (MSCs), the formative pluripotent blast cells, give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the BM far exceeds their abundance in other tissues and as such isolation from BM is presently preferred. Adult tissue stem cells can be isolated using various methods known in the art such as those disclosed by Alison, M. R. [J Pathol. (2003) 200(5): 547-50]. Fetal stem cells can be isolated using various methods known in the art such as those disclosed by Eventov-Friedman S, et al. [PLoS Med. (2006) 3: e215].

Hematopoietic stem cells can be isolated using various methods known in the arts such as those disclosed by “Handbook of Stem Cells” edit by Robert Lanze, Elsevier Academic Press, 2004, Chapter 54, pp 609-614, isolation and characterization of hematopoietic stem cells, by Gerald J Spangrude and William B Stayton.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

“Artificial” when used with reference to polynucleotide sequence means a sequence not found in nature which is, for example, a synthetic modification of a natural sequence, or contains an unnatural sequence.

### Inhibitory RNA and Polynucleotide

The term “inhibitory RNA” refers to an RNA molecule having a degrading or silencing, e.g. partial or full silencing, effect on its corresponding messenger RNA having a complementary sequence thereto. In some embodiments of the invention, the inhibitory RNA include siRNAs, shRNAs and microRNAs, preferably microRNAs.

According to one embodiment, the inhibitory RNA molecule is a capable of inducing RNA interference (RNAi), optionally after cellular processing.

According to some embodiments, the inhibitory RNA molecule is processed from a precursor.

According to one embodiment, the non-coding RNA molecule or RNA silencing molecule is processed from a single stranded RNA (ssRNA) precursor.

According to one embodiment, the non-coding RNA molecule or the RNA silencing molecule is processed from a duplex-structured single-stranded RNA precursor.

According to one embodiment, the non-coding RNA molecule or RNA silencing molecule is processed from a dsRNA precursor (e.g. comprising perfect and imperfect base pairing).

According to one embodiment, the non-coding RNA molecule or the RNA silencing molecule is processed from a non-structured RNA precursor.

According to one embodiment, the non-coding RNA molecule or the RNA silencing molecule is processed from a protein-coding RNA precursor.

According to one embodiment, the non-coding RNA molecule or the RNA silencing molecule is processed from a non-coding RNA precursor.

According to one embodiment, the dsRNA can be derived from two different complementary RNAs, or from a single RNA that folds on itself to form dsRNA.

Perfect and imperfect based paired RNA (i.e. double stranded RNA, dsRNA), siRNA and shRNA—The presence of long dsRNAs in cells stimulates the activity of a ribonuclease Ill enzyme referred to as dicer. Dicer, also known as endoribonuclease Dicer or helicase with RNase motif, is an enzyme that in humans is encoded by the DICER 1 gene. Dicer is involved in the processing of the dsRNA into short pieces of dsRNA known as short interfering RNAs (siRNAs). siRNAs derived from dicer activity are typically about 21 to about 23 nucleotides in length and comprise about 19 base pair duplexes with two 3′ nucleotides overhangs.

Accordingly, some embodiments of the invention contemplate modifying a genomic sequence encoding a dsRNA to redirect a silencing specificity (including silencing activity) towards a B2M mRNA.

According to one embodiment dsRNA precursors longer than 21 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13: 115-125; Diallo M., et al., Oligonucleotides. 2003; 13:381-392; Paddison P. J., et al., Proc. Natl Acad. Sci. USA. 2002; 99: 1443-1448; Tran N., et al., FEBS Lett. 2004; 573: 127-134].

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position, but not the composition, of the 3′-overhang influences potency of a siRNA and asymmetric duplexes having a 3′-overhang on the antisense strand are generally more potent than those with the 3′-overhang on the sense strand (Rose et al., 2005).

The strands of a double-stranded interfering RNA (e.g., a siRNA) may be connected to form a hairpin or stem-loop structure (e.g., a shRNA). Thus, as mentioned, the inhibitory RNA of some embodiments of the invention may also be a short hairpin RNA (shRNA).

The term short hairpin RNA, “shRNA”, as used herein, refers to a RNA molecule having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

The inhibitory RNA molecule of some embodiments of the invention need not be limited to those molecules containing only RNA, but further encompasses chemically-modified nucleotides and non-nucleotides.

Various types of siRNAs are contemplated by the present invention, including trans-acting siRNAs (Ta-siRNAs), repeat-associated siRNAs (Ra-siRNAs) and natural-antisense transcript-derived siRNAs (Nat-siRNAs).

According to one embodiment, silencing RNA includes “piRNA” which is a class of Piwi-interacting RNAs of about 26 and 31 nucleotides in length. piRNAs typically form RNA-protein complexes through interactions with Piwi proteins, i.e. antisense piRNAs are typically loaded into Piwi proteins (e.g. Piwi, Ago3 and Aubergine (Aub)).

miRNA—According to another, typically preferred, embodiment the inhibitory RNA molecule may be a miRNA. The term “microRNA”, “miRNA”, and “miR” are synonymous and refer to a collection of non-coding single-stranded RNA molecules of about 19-28 nucleotides in length, which regulate gene expression. miRNAs are found in a wide range of organisms, including viruses, and have been shown to play a role in development, homeostasis, and disease etiology.

Initially the pre-miRNA is present as a long non-perfect double-stranded stem loop RNA that is further processed by Dicer into a siRNA-like duplex, comprising the mature guide strand (miRNA) and a similar-sized fragment known as the passenger strand (miRNA*). The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC. When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-8 of the miRNA (referred as “seed sequence”).

A number of studies have looked at the base-pairing requirement between miRNA and its mRNA target for achieving efficient inhibition of translation (reviewed by Bartel 2004, Cell 116-281). Computational studies, analyzing miRNA binding on whole genomes have suggested a specific role for bases 2-8 at the 5′ of the miRNA (also referred to as “seed sequence”) in target binding but the role of the first nucleotide, found usually to be “A” was also recognized (Lewis et al 2005 Cell 120-15). Similarly, nucleotides 1-7 or 2-8 were used to identify and validate targets by Krek et al. (2005, Nat Genet 37-495). The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer. It will be appreciated that the pre-miRNA sequence may comprise from 45-90, 60-80 or 60-70 nucleotides while the pri-miRNA sequence may comprise from 45-30,000, 50-25,000, 100-20,000, 1,000-1,500 or 80-100 nucleotides

The terms “polynucleotide”, “nucleotide” or “nucleic acid” are used interchangeably herein and refer to a polymeric macromolecule made from nucleotide monomers particularly deoxyribonucleotide or ribonucleotide monomers chain of nucleotides, regardless of length. Nucleotides include species that comprise purines, e.g., adenine, hypoxanthine, guanine, and their derivatives and analogs, as well as pyrimidines, e.g., cytosine, uracil, thymine, and their derivatives and analogs. The term also encompasses nucleic acids containing known nucleotide analogs or modified backbone residues or linkages, which are naturally occurring and non-naturally occurring, which have similar properties as the reference nucleic acid, and which are intended to be metabolized in a manner similar to the reference nucleotides or are intended to have extended half-life in the system. Examples of such analogs include, without limitation, phosphorothioates, phosphoramidates, methyl phosphonates, chiral-methyl phosphonates, 2-O-methyl ribonucleotides, peptide-nucleic acids (PNAs). Suitably the term “polynucleotide” refers to naturally occurring polymers of deoxyribonucleotide or ribonucleotide monomers. Non-limiting examples of polynucleotides include a gene, a gene fragment, messenger RNA (mRNA), complementary DNA (cDNA), non-coding RNA (ncRNA), microRNA (miRNA), small RNA (sRNA), short interfering RNA (siRNA), short hairpin RNA (shRNA), recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. Suitably the polynucleotides of the invention are recombinant. Recombinant means that the polynucleotide is the product of at least one of cloning, restriction or ligation step, or other procedures that result in a nucleic acid molecule that is distinct from a nucleic acid molecule found in nature (e.g. in the case of cDNA).

The present invention thus in some aspects relates to an inhibitory RNA or a polynucleotide sequence encoding an inhibitory RNA, preferably a microRNA, comprised of a modified sequence of an endogenous pre-miRNA (hairpin), also referred to as the endogenous miRNA scaffold, modified to encode a novel silencing RNAi trigger instead of its native guide strand. The novel polynucleotides match a corresponding messenger RNA (mRNA) by sequence complementarity (e.g. B2M mRNA) leading to the silencing of B2M through the RNA interference pathway.

Thus, the invention provides for an inhibitory RNA, or a polynucleotide sequence encoding an inhibitory RNA, preferably a microRNA, the inhibitory RNA comprising or consisting a sequence selected from:
(i) SEQ ID NOs: 1-17, 64-127;
(ii) a variant of SEQ ID NOs: 1-17, 64-127;
(iii) a fragment of SEQ ID NOs: 1-17, 64-127; and
(iv) a DNA sequence complementary to any one of (i), (ii) or (iii).

The invention also provides for an inhibitory, or a polynucleotide sequence encoding an inhibitory RNA, preferably a microRNA, the inhibitory RNA comprising or consisting a sequence selected from:
(i) any one of SEQ ID NOs: 18-34, 128-191;
(ii) a variant of any one of SEQ ID NOs: 18-34, 128-191
(iii) a fragment of any one of SEQ ID NOs: 18-34, 128-191; and
(iv) a DNA sequence complementary to any one of (i), (ii) or (iii).

The invention further provides for a polynucleotide sequence encoding an inhibitory RNA comprising or consisting a sequence selected from:
(i) any one of SEQ ID NOs: 35-51;
(ii) a variant of any one of SEQ ID NOs: 35-51; and
(iii) a fragment of any one of SEQ ID NOs: 35-51.

The inhibitory RNAs or polynucleotides encoding the inhibitory RNAs of the present invention are capable of silencing, degrading, reducing or modifying the functional activity of the target RNA or target mRNA, suitably wherein the target mRNA is translatable to B2M. The inhibitory RNAs may be fully complementary to the target, or may harbour deliberately designed mismatches.

In some embodiments, the inhibitory RNAs or polynucleotides encoding the inhibitory RNAs of the invention silence, reduce or modify the functional activity of the target RNA or target mRNA by at least 10%, at least 25%, at least 50%, at least 75% or at least 90% or at least 95% compared to the functional activity of the target RNA or target mRNA in the absence of the inhibitory RNAs or polynucleotides encoding the inhibitory RNAs of the invention. More preferably, the inhibitory RNAs or polynucleotides encoding the inhibitory RNAs of the invention silence, degrade, reduce or modify the functional activity of the target RNA or target mRNA by at least 1%, at least 5%, at least 15%, at least 20% or at least 80%, at least 85% or at least 95% or at least 99% compared to the functional activity of the target RNA or target mRNA in the absence of the inhibitory RNAs or polynucleotides encoding the inhibitory RNAs of the invention.

In some preferred embodiments, the inhibitory RNAs or polynucleotides encoding the inhibitory RNAs of the invention silence, reduce or modify the functional activity of B2M by at least 30% (e.g. Solution 30, SEQ ID NO: 10 and SEQ ID NO: 27). In some preferred embodiments, the inhibitory RNAs or polynucleotides encoding the inhibitory RNAs of the invention silence, reduce or modify the functional activity of B2M by at least 65% (e.g. Solution 20, SEQ ID NO: 5 and SEQ ID NO: 22). In some preferred embodiments, the inhibitory RNAs or polynucleotides encoding the inhibitory RNAs of the invention silence, reduce or modify the functional activity of B2M by at least 85%, preferably by at least 95% (e.g. Solution 29, SEQ ID NO: 9 and SEQ ID NO: 26). In some preferred embodiments, the inhibitory RNAs or polynucleotides encoding the inhibitory RNAs of the invention silence, reduce or modify the functional activity of B2M by at least 80% (e.g. Solution 30, SEQ ID NO: 10 and SEQ ID NO: 27). In some embodiments, the reduction or modification of functional activity is observed in myeloid cells. In some embodiments, the reduction or modification of functional activity is observed in pancreatic cells. In some embodiments, the reduction or modification of functional activity is observed in CD4+ and CD8+ T cells.

The present invention also relates to variants of the inhibitory RNAs or polynucleotides encoding the inhibitory RNAs of the invention. In general, variants of the inhibitory RNAs or polynucleotides encoding the inhibitory RNAs of the invention include sequences having a high degree of sequence identity thereto. For example variants suitably have at least about 50%, at least about 80% sequence identity, more preferably at least about 85% sequence identity and most preferably at least about 90% sequence identity (such as at least about 95%, at least about 98% or at least about 99%) to the associated reference sequence over their whole length, i.e. SEQ ID NOs: 1-51, 64-191. The variants may preferably have at least 80%, 81%, 82%, 83%, 84%, 85%, 86%, 87%, 88%, 89%, 90%, 91%, 92%, 93%, 94%, 95%, 96%, 97%, 98% or 99% sequence identity to the reference sequence over their whole length, i.e. SEQ ID NOs: 1-51, 64-191. The variant may be identical to the reference sequence, i.e. SEQ ID NOs: 1-51, 64-191, apart from the inclusion of one or more specific modifications. Identity relative to the sequence of SEQ ID NOs: 1-51, 64-191 can be measured over a region of at least 5, at least 10, at least 20, at least 50 or more contiguous nucleotides of the sequence of SEQ ID NOs: 1-51, 64-191, or more preferably over the full length of SEQ ID NOs: 1-51, 64-191.

Suitably the variant is an active variant. A variant is considered to be an active variant where it elicits an effect which is at least 20%, suitably at least 50% and especially at least 75% (such as at least 90%, 95% or 99%) of the activity of the reference sequence (i.e. the sequence of which the variant is a variant) in any suitable test assay, such as those described in the examples below.

The sequence of the inhibitory RNAs or polynucleotides encoding the inhibitory RNAs of the invention may comprise a variant of the polynucleotide sequence of SEQ ID NOs: 1-51, 64-191 in which modifications, such as nucleotide additions, deletions or substitutions are made relative to the sequence of any one of SEQ ID NOs: 1-51, 64-191. The variant may, for example, be a conservatively modified variant. A “conservatively modified variant” is one where the alteration(s) results in the substitution/deletion/addition of residues which do not substantially impact the biological function of the variant. Typically, such biological function of the variants will be to induce a silencing of B2M protein expression. Variants can include homologues of polynucleotides found in other species.

In some embodiments, a variant of the inhibitory RNAs or polynucleotides encoding the inhibitory RNAs of the invention contains a number of substitutions, for example, conservative substitutions (for example, 1-25, such as 1-10, in particular 1-5, and especially 1 nucleotide may be altered) when compared to the reference sequence. The number of substitutions, for example, conservative substitutions, may be up to 20% e.g., up to 10% e.g., up to 5% e.g., up to 1% of the number of nucleotides of the reference sequence.

Fragments according to the present invention will typically comprise at least 9 contiguous nucleotides from the full-length polynucleotide sequence (e.g., at least 9 or 10), such as at least 12 contiguous nucleotides (e.g., at least 15 or at least 20 contiguous nucleotides), in particular at least 50 or more contiguous nucleotides depending on the length of the polynucleotide reference sequence. In some embodiments, the fragments suitably will be at least 10%, such as at least 20%, such as at least 50%, such as at least 70% or at least 80% of the length of the full-length polynucleotide sequence.

In one embodiment of the invention, the polynucleotides are RNA. In an alternative embodiment, the polynucleotides of the invention are DNA. In a further embodiment of the invention, the polynucleotides of the invention are synthetic nucleic acid sequences (e.g., a polynucleotide sequence with a non-naturally occurring sequence).

DNA (deoxyribonucleic acid) and RNA (ribounucleic acid) refer to nucleic acid molecules having a backbone of sugar moieties which are deoxyribosyl and ribosyl moieties respectively. The sugar moieties may be linked to bases which are the 4 natural bases (adenine (A), guanine (G), cytosine (C) and thymine (T) in DNA and adenine (A), guanine (G), cytosine (C) and uracil (U) in RNA). A “corresponding RNA” is an RNA having the same sequence as a reference DNA but for the substitution of thymine (T) in the DNA with uracil (U) in the RNA. The sugar moieties may also be linked to unnatural bases such as inosine, xanthosine, 7-methylguanosine, dihydrouridine and 5-methylcytidine. Natural phosphodiester linkages between sugar (deoxyribosyl/ribosyl) moieties may optionally be replaced with phosphorothioates linkages. Suitably nucleic acids of the invention consist of the natural bases attached to a deoxyribosyl or ribosyl sugar backbone with phosphodiester linkages between the sugar moieties. In an embodiment the nucleic acid of the invention is a DNA. For example the nucleic acid comprises or consists of a sequence selected from SEQ ID NOs: 1-34, 64-191.

In one embodiment of the invention, the polynucleotide further comprises an miRNA scaffold. The miRNA scaffold may consist of at least 50, at least 100 or at least 150 nucleotide bases. The inhibitory RNAs or polynucleotides encoding the inhibitory RNAs of the invention are comprised of a modified sequence of an endogenous pre-miRNA (hairpin), also referred to herein as an “endogenous miRNA scaffold,” “scaffold portion,” or simply “scaffold”. The nucleotide bases of the miRNA scaffold may flank both sides of a novel polynucleotide sequence of the invention, i.e. SEQ ID NOs: 1-17, 64-127.

Thus, in one aspect, the invention provides miRNA scaffolds useful for the generation of non-naturally occurring miRNAs. A non-naturally occurring miRNA of the disclosure comprises a miRNA scaffold derived from (i.e. at least 60% identical to, at least 70% identical, at least 80% identical, at least 85% identical, at least 90% identical, at least 95% identical, up to and including 100% identical to) a specific endogenous miRNA. A single miRNA scaffold of the disclosure can be used to provide an almost unlimited number of different non-naturally occurring miRNAs. Endogenous miRNAs from which the miRNA scaffold sequences of the disclosure are derived include, but are not limited to, eukaryotic miRNA scaffold sequences, preferably a mammalian miRNA scaffold sequence, more preferably a human pre-miRNA scaffold. In some embodiments of the invention, the human pre-microRNA scaffold is selected from any one of hsa-mir-191; hsa-mir-302a; hsa-mir-302c; hsa-mir-93; hsa-mir-106a; hsa-mir-106b; hsa-mir-200c; hsa-mir-20a; hsa-mir-21; hsa-mir-363; hsa-mir-518b; hsa-mir-744; hsa-mir-99b; hsa-mir-320a; hsa-mir-520f; hsa-mir-652; hsa-mir-1180; hsa-mir-15b; hsa-mir-182; hsa-mir-23a; hsa-mir-26b; hsa-mir-335; hsa-mir-361; hsa-mir-1307; hsa-mir-205; hsa-mir-22; hsa-mir-221; hsa-mir-222; hsa-mir-30e; hsa-mir-423; hsa-mir-519c; hsa-mir-92b; hsa-mir-30; hsa-mir-302b; hsa-mir-375; hsa-let-7b; hsa-let-7g; and hsa-mir-98.

In some embodiments of the invention, the inhibitory RNA or polynucleotide sequence encoding an inhibitory RNA encoded by any one of SEQ ID Nos: 1-17, 64-127 is inserted into a human pre-microRNA sequence. Preferably, SEQ ID NO: 1 or 7 is inserted into hsa-mir-191 (SEQ ID NO: 18 or 24, respectively); SEQ ID NO: 2 or 11 is inserted into hsa-mir-302a (SEQ ID NO: 19 or 28, respectively); SEQ ID NO: 3 or 12 is inserted into hsa-mir-302c (SEQ ID NO: 20 or 29, respectively); SEQ ID NO: 4 or 16 is inserted into hsa-mir-93 (SEQ ID NO: 21 or 33, respectively); SEQ ID NO: 5 is inserted into hsa-mir-106a (SEQ ID NO: 22); SEQ ID NO: 6 is inserted into hsa-mir-106b (SEQ ID NO: 23); SEQ ID NO: 8 is inserted into hsa-mir-200c (SEQ ID NO: 25); SEQ ID NO: 9 is inserted into hsa-mir-20a (SEQ ID NO: 26); SEQ ID NO: 10 is inserted into hsa-mir-21 (SEQ ID NO: 27); SEQ ID NO: 13 is inserted into hsa-mir-363 (SEQ ID NO: 30); SEQ ID NO: 14 is inserted into hsa-mir-518b (SEQ ID NO: 31); SEQ ID NO: 15 is inserted into hsa-mir-744 (SEQ ID NO: 32); and SEQ ID NO: 17 is inserted into hsa-mir-99b (SEQ ID NO: 34). Preferably, SEQ ID NO: 64, SEQ ID NO: 73 or SEQ ID NO: 89 is inserted into hsa-mir-320a (SEQ ID NO: 128, SEQ ID NO: 137 or SEQ ID NO: 153 respectively). Preferably, SEQ ID NO: 65, SEQ ID NO: 77 or SEQ ID NO: 94 is inserted into hsa-mir-520f (SEQ ID NO: 129, SEQ ID NO: 141 or SEQ ID NO: 158 respectively). Preferably, SEQ ID NO: 66, SEQ ID NO: 78 or SEQ ID NO: 95 is inserted into hsa-mir-652 (SEQ ID NO: 130, SEQ ID NO: 142 or SEQ ID NO: 159 respectively). Preferably, SEQ ID NO: 67 is inserted into hsa-mir-106a (SEQ ID NO: 131). Preferably, SEQ ID NO: 68 or SEQ ID NO: 79 is inserted into hsa-mir-1180 (SEQ ID NO: 132 or SEQ ID NO: 143 respectively). Preferably, SEQ ID NO: 69 or SEQ ID NO: 81 is inserted into hsa-mir-15b (SEQ ID NO: 133 or SEQ ID NO: 145 respectively). Preferably, SEQ ID NO: 70, SEQ ID NO: 82 or SEQ ID NO: 99 is inserted into hsa-mir-182 (SEQ ID NO: 134, SEQ ID NO: 146 or SEQ ID NO: 163 respectively). Preferably, SEQ ID NO: 71 or SEQ ID NO: 87 is inserted into hsa-mir-23a (SEQ ID NO:135 or SEQ ID NO: 151 respectively). Preferably, SEQ ID NO: 72 is inserted into hsa-mir-26b (SEQ ID NO: 136). Preferably, SEQ ID NO: 74 or SEQ ID NO: 90 is inserted into hsa-mir-335 (SEQ ID NO: 138 or SEQ ID NO: 154 respectively). Preferably, SEQ ID NO: 75 or SEQ ID NO: 91 is inserted into hsa-mir-361 (SEQ ID NO: 139 or SEQ ID NO: 155 respectively). Preferably, SEQ ID NO: 76 is inserted into hsa-mir-518b (SEQ ID NO: 140). Preferably, SEQ ID NO: 80 is inserted into hsa-mir-1307 (SEQ ID NO: 144). Preferably, SEQ ID NO: 83 is inserted into hsa-mir-205 (SEQ ID NO: 147). Preferably, SEQ ID NO: 84 is inserted into hsa-mir-22 (SEQ ID NO: 148). Preferably, SEQ ID NO: 85 is inserted into hsa-mir-221 (SEQ ID NO: 149). Preferably, SEQ ID NO: 86 is inserted into hsa-mir-222 (SEQ ID NO: 150). Preferably, SEQ ID NO: 88 is inserted into hsa-mir-30e (SEQ ID NO: 152). Preferably, SEQ ID NO: 92 is inserted into hsa-mir-423 (SEQ ID NO: 156). Preferably, SEQ ID NO: 93 is inserted into hsa-mir-519c (SEQ ID NO: 157). Preferably, SEQ ID NO: 96 is inserted into hsa-mir-92b (SEQ ID NO: 160). Preferably, SEQ ID NO: 97, SEQ ID NO: 103, SEQ ID NO: 104 or SEQ ID NO: 109 is inserted into hsa-mir-302a (SEQ ID NO: 161, SEQ ID NO: 167, SEQ ID NO: 168 or SEQ ID NO: 173 respectively). Preferably, SEQ ID NO: 98, SEQ ID NO: 107, SEQ ID NO: 108 or SEQ ID NO: 111 is inserted into hsa-mir-302c (SEQ ID NO: 162, SEQ ID NO: 171, SEQ ID NO: 172 or SEQ ID NO: 175 respectively). Preferably, SEQ ID NO: 100 is inserted into hsa-mir-191 (SEQ ID NO: 164). Preferably, SEQ ID NO: 101 or SEQ ID NO: 102 is inserted into hsa-mir-30 (SEQ ID NO: 165 or SEQ ID NO: 166 respectively). Preferably, SEQ ID NO: 105, SEQ ID NO: 106 or SEQ ID NO: 110 is inserted into hsa-mir-302b (SEQ ID NO: 169, SEQ ID NO: 170 or SEQ ID NO: 174 respectively). Preferably, SEQ ID NO: 112, SEQ ID NO: 113 or SEQ ID NO: 114 is inserted into hsa-mir-375 (SEQ ID NO: 176, SEQ ID NO: 177 or SEQ ID NO: 178 respectively). Preferably, SEQ ID NO: 115, SEQ ID NO: 119, SEQ ID NO: 122 or SEQ ID NO: 125 is inserted into hsa-let-7b (SEQ ID NO: 179, SEQ ID NO: 183, SEQ ID NO: 186 or SEQ ID NO: 189 respectively). Preferably, SEQ ID NO: 116, SEQ ID NO: 120, SEQ ID NO: 123 or SEQ ID NO: 126 is inserted into hsa-let-7g (SEQ ID NO: 180, SEQ ID NO: 184, SEQ ID NO: 187 or SEQ ID NO: 190 respectively). Preferably, SEQ ID NO: 117, SEQ ID NO: 118, SEQ ID NO: 121, SEQ ID NO: 124 or SEQ ID NO: 127 is inserted into hsa-mir-98 (SEQ ID NO: 181, SEQ ID NO: 182, SEQ ID NO: 185, SEQ ID NO: 188 or SEQ ID NO: 191 respectively).

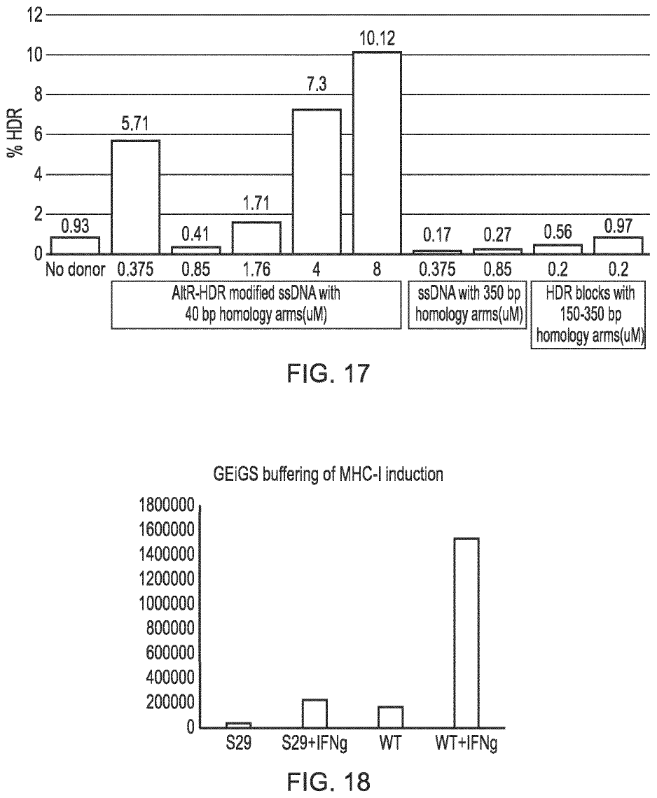

In some preferred embodiments, the polynucleotide sequence encoding an inhibitory RNA comprises a genomic sequence flanking both sides of a human pre-microRNA. In some preferred embodiments, the genomic sequence flanking both sides of a human pre-microRNA has a length of approximately 40 bp. In some preferred embodiments, the genomic sequence flanking both sides of a human pre-microRNA has a length of less than 100 bp, less than 90 bp, less than 80 bp, less than 70 bp, preferably less than 60 bp, less than 50 bp, more preferably less than 49 bp, less than 48 bp, less than 47 bp, less than 46 bp, less than 45 bp, less than 44 bp, less than 43 bp, less than 42 bp, less than 41 bp. Flanking genomic sequences (homology arms) of length 40 base pairs show superior silencing potency compared to flanking genomic sequences of length 350 bp or length 150-350 bp, as shown in FIG. 17.

In another embodiment of the invention, the polynucleotide sequence encoding an inhibitory RNA comprises a genomic sequence flanking both sides of a human pre-microRNA suitably wherein the polynucleotide sequence comprises:
(i) SEQ ID NO: 35 (which comprises SEQ ID NO: 2 or SEQ ID NO: 19);
(ii) SEQ ID NO: 36 (which comprises SEQ ID NO: 11 or SEQ ID NO: 28);
(iii) SEQ ID NO: 37 (which comprises SEQ ID NO: 3 or SEQ ID NO: 20);
(iv) SEQ ID NO: 38 (which comprises SEQ ID NO: 12 or SEQ ID NO: 29);
(v) SEQ ID NO: 39 (which comprises SEQ ID NO: 9 or SEQ ID NO: 26);
(vi) SEQ ID NO: 40 (which comprises SEQ ID NO: 8 or SEQ ID NO: 25);
(vii) SEQ ID NO: 41 (which comprises SEQ ID NO: 10 or SEQ ID NO: 27);
(viii) SEQ ID NO: 42 (which comprises SEQ ID NO: 13 or SEQ ID NO: 30);
(iv) SEQ ID NO: 43 (which comprises SEQ ID NO: 4 or SEQ ID NO: 21);
(x) SEQ ID NO: 44 (which comprises SEQ ID NO: 16 or SEQ ID NO: 33);
(xi) SEQ ID NO: 45 (which comprises SEQ ID NO: 5 or SEQ ID NO: 22;
(xii) SEQ ID NO: 46 (which comprises SEQ ID NO: 6 or SEQ ID NO: 23;
(xiii) SEQ ID NO: 47 (which comprises SEQ ID NO: 1 or SEQ ID NO: 18;
(xvi) SEQ ID NO: 48 (which comprises SEQ ID NO: 7 or SEQ ID NO: 24;
(xv) SEQ ID NO: 49 (which comprises SEQ ID NO: 15 or SEQ ID NO: 32;
(xvi) SEQ ID NO: 50 (which comprises SEQ ID NO: 14 or SEQ ID NO: 31; or
(xvii) SEQ ID NO: 51 (which comprises SEQ ID NO: 17 or SEQ ID NO: 34).

In yet a further embodiment of the invention, the inhibitory RNA or polynucleotide sequence encoding an inhibitory RNA according to the invention, wherein the inhibitory RNA targets an exon sequence comprised in the messenger RNA from the B2M gene, suitably exon 2-1, 2-2 or exon 4, preferably wherein the inhibitory RNA comprises:
(i) SEQ ID NO: 2 or SEQ ID NO: 19, which targets B2M gene exon 2-2;
(ii) SEQ ID NO: 11 or SEQ ID NO: 28, which targets B2M gene exon 4;
(iii) SEQ ID NO: 3 or SEQ ID NO: 20, which targets B2M gene exon 2-2;
(iv) SEQ ID NO: 12 or SEQ ID NO: 29, which targets B2M gene exon 4;
(v) SEQ ID NO: 9 or SEQ ID NO: 26, which targets B2M gene exon 4;
(vi) SEQ ID NO: 8 or SEQ ID NO: 25, which targets B2M gene exon 4;
(vii) SEQ ID NO: 10 or SEQ ID NO: 27, which targets B2M gene exon 4;
(viii) SEQ ID NO: 13 or SEQ ID NO: 30, which targets B2M gene exon 4;
(iv) SEQ ID NO: 4 or SEQ ID NO: 21 which targets B2M gene exon 2-2;
(x) SEQ ID NO: 16 or SEQ ID NO: 33, which targets B2M gene exon 4;
(xi) SEQ ID NO: 5 or SEQ ID NO: 22, which targets B2M gene exon 4;
(xii) SEQ ID NO: 6 or SEQ ID NO: 23, which targets B2M gene exon 4;
(xiii) SEQ ID NO: 1 or SEQ ID NO: 18, which targets B2M gene exon 2-2;
(xvi) SEQ ID NO: 7 or SEQ ID NO: 24, which targets B2M gene exon 4;
(xv) SEQ ID NO: 15 or SEQ ID NO: 32, which targets B2M gene exon 4;
(xvi) SEQ ID NO: 14 or SEQ ID NO: 31, which targets B2M gene exon 4;
(xvii) SEQ ID NO: 17 or SEQ ID NO: 34, which targets B2M gene exon 4;
(xviii) SEQ ID NOs: 64-66 or SEQ ID NOs: 128-130, which target B2M exon 2-1;
(xix) SEQ ID NOs: 67-78 or SEQ ID NOs: 129-142, which target B2M exon 2-2; and/or
(xx) SEQ ID NOs: 79-96 or SEQ ID NOs: 143-160, which target B2M exon 4.

The polynucleotide sequence encoding the inhibitory RNA may be introduced into the cell via any method known to the skilled person. In some embodiments, the nucleic acid construct adapted to express an inhibitory RNA suitable to inhibit expression of the target gene is introduced via lentiviral delivery.

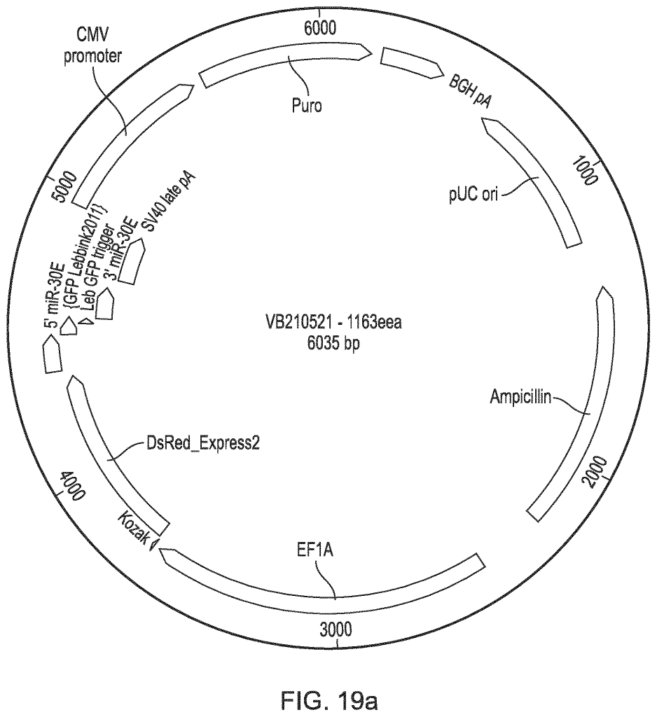

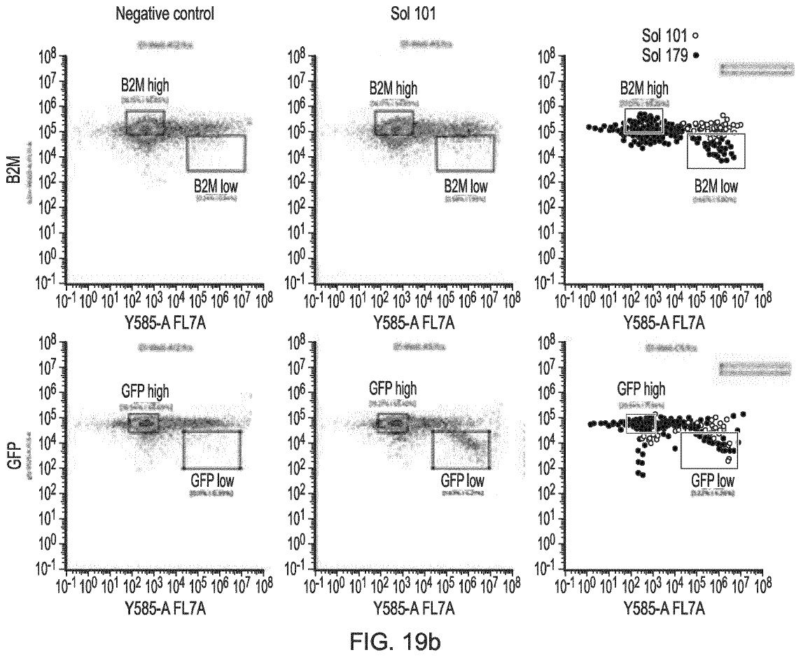

The inhibitory RNA, or the polynucleotide sequence encoding the inhibitory RNA according to the present invention are particularly suitable for treatment of chronic inflammatory conditions as they can buffer the MHC-I induction by inflammatory stimuli as shown in FIG. 18 and Example 13 below.

Previous methods utilising shRNA or siRNA are known to have significant off-target effects as shown in Rao, et al. which is incorporated herein by reference [Comparative assessment of siRNA and shRNA off target effects: what is slowing clinical development. Rao D D, Senzer N, Cleary M A, Nemunaitis J. Cancer Gene Ther. 2009 November; 16(11):807-9. Doi: 10.1038/cgt.2009.53. Epub 2009 Aug. 28. PMID: 19713999]. Advantageously, in some preferred embodiments, the inhibitory RNA, or the polynucleotide sequence encoding the inhibitory RNA according to the present invention does not have off-target effects. In some preferred embodiments, the inhibitory RNA, or the polynucleotide sequence encoding the inhibitory RNA does not silence unintended targets (any target other than the target it is designed to target such as B2M). In some preferred embodiments, the inhibitory RNA, or the polynucleotide sequence encoding the inhibitory RNA does not show loss-of-function effects following redirection of miRNA genes used as scaffolds.

### Expression Cassettes and Vectors

Polynucleotides can be synthesised according to methods well known in the art. The polynucleotide sequence encoding an inhibitory RNA of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polynucleotide of the invention.

The present invention, in some embodiments, includes expression vectors that comprise such polynucleotide sequences encoding an inhibitory RNA of the present invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a polynucleotide of the invention. Other suitable vectors would be apparent to persons skilled in the art.

In some embodiments, the expression cassette may be a synthetic expression cassette. In other embodiments, the expression cassette disclosed herein comprises a polynucleotide sequence encoding an inhibitory RNA operably linked to one or more regulatory elements suitable for permitting transcription of the inhibitory RNA in a cell, preferably a promoter, optionally a constitutive promoter or an inducible promoter. In other embodiments of the invention, the expression cassette comprises a promoter operably linked to a sequence encoding an expression product, suitably a polynucleotide sequence (e.g. a miRNA or a transgene such as a therapeutic transgene). Alternatively, the expression cassette disclosed herein may comprise an inducible promoter operably linked to a sequence encoding an expression product, suitably a polynucleotide sequence (e.g. a miRNA or a transgene such as a therapeutic transgene). Preferably, the promoter is EF1alpha.

In some embodiments, the expression product is a therapeutic expression product. In some preferred embodiments, the therapeutic expression product is suitable for use in treating a disease or condition. In some embodiments, the disease or condition is an autoimmune disease, optionally Type I diabetes, or cancer. In a preferred embodiments, therapeutic expression products include those useful in the treatment of disease, preferably Type I diabetes, or cancer.

Suitably the expression cassette comprises sequences providing or coding for one or more of, and preferably all of, a ribosomal binding site, a start codon, a stop codon, and a transcription termination sequence. Suitably the expression cassette comprises a nucleic acid encoding a posttranscriptional regulatory element. Suitably the expression cassette comprises a nucleic acid encoding a polyA element.

A vector comprising a polynucleotide sequence encoding an inhibitory RNA or an expression cassette according to the present invention is disclosed herein. In one embodiment of the invention, the vector comprises DNA encoding regulatory elements for permitting transcription of a non-coding RNA molecule in a cell. The regulatory element may be positioned with respect to an expressible nucleotide sequence such that the element can affect its regulatory activity, i.e. the regulatory element is “operably linked”. Alternatively, the regulatory element may be a transcriptional regulatory element having enhancer activity, for example, can be located at some distance, including adjacent to or a number of nucleotides away from, and upstream or downstream from a promoter and a nucleotides sequence to be transcribed, and still exert a detectable enhancing effect on the level of expression of an encoded reporter molecule. The regulatory element, including eukaryotic and prokaryotic promoters, terminators, enhancers, and silencers, are well known in the art and can be chemically synthesized, obtained from naturally occurring nucleic acid molecules, or purchased from commercial sources. In an embodiment of the invention, the DNA encoding regulatory elements for permitting transcription are of a non-coding RNA, preferably a miRNA, in a cell, preferably a eurkaryotic cell, more preferably a mammalian cell, for example a human cell or an iPSC cell.

The vector may be a gene or mRNA therapy vector. In some embodiments of the invention, the vector is a viral vector, preferably a lentiviral, adenoviral, adeno-associated virus (AAV) or retroviral vector. The AAV may be selected from the group consisting of: AAV2, AAV5, AAV6, AAV7, AAV8, AAV9 BNP116, rh10, AAV2.5, AAV2i8, AAVDJ8 and AAV2G9, or derivatives thereof. AAV vectors may be used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. The AAV vector may be chimeric, meaning it comprises components from at least two AAV serotypes, such as the ITRs of an AAV2 and the capsid protein of an AAV5. In other embodiments of the invention, the vector is a viral vector, preferably an alphavirus, herpes virus, arena virus, measles virus, poxvirus or paramyxovirus vector. In some embodiments of the invention, the vector is a plasmid. Such a plasmid may include a variety of other functional nucleic acid sequences, such as one or more selectable markers, one or more origins of replication, multiple cloning sites and the like. In some embodiments the plasmid is plasmid VB210602-1567ytv from VectorBuilder.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian adenoviral vectors, the pSV and the pCMV series of vectors are particularly well-known non-limiting examples. There are many well-known yeast expression vectors including, without limitation, yeast integrative plasmids (Yip) and yeast replicative plasmids (Yrp).

The invention further provides recombinant virions (viral particles) comprising a vector as described above.

The expression cassette according to the aspects of the present invention may comprise polynucleotide sequence encoding two or more inhibitory RNAs according to the invention. The inhibitory RNAs may be inhibitory RNAs disclosed herein or other inhibitory RNAs. In some embodiments, the two or more inhibitory RNAs have the same target (e.g. two or more inhibitory RNAs with the same target, such as B2M). In some embodiments, the two or more inhibitory RNAs may comprise or consist of a sequence selected from SEQ ID Nos: 1-17, 64-127 or functional variants or fragments thereof. In some embodiments, the two or more inhibitory RNAs may comprise or consist of a sequence selected from SEQ ID Nos: 1-17 or functional variants or fragments thereof. In some embodiments, the two or more inhibitory RNAs may comprise or consist of a sequence selected from SEQ ID Nos: 64-127. In some embodiments, the two or more inhibitory RNAs may comprise or consist of a sequence selected from SEQ ID Nos: 18-34, 128-191, or functional variants or fragments thereof. In some embodiments, the two or more inhibitory RNAs may comprise or consist of a sequence selected from SEQ ID Nos: 18-34. In some embodiments, the two or more inhibitory RNAs may comprise or consist of a sequence selected from SEQ ID Nos: 128-191. In some embodiments, the two or more inhibitory RNAs have different targets.

The expression cassettes, vectors and virions according to the present invention are particularly suitable for treatment of chronic inflammatory conditions, particularly ex vivo cell therapy, as they can buffer the augmentation of MHC-I expression in ex vivo therapeutic cells as the cells are exposed to inflammatory stimuli as shown in FIG. 18 and Example 13 below.

Synthetic microRNA

In one embodiment of the invention, there is provided a synthetic microRNA comprising or consisting of a sequence selected from SEQ ID Nos: 1-17, 64-127 or functional variants or fragments thereof. In a further embodiment of the invention, there is provided a synthetic pre-microRNA comprising or consisting of a sequence selected from SEQ ID Nos: 1-17, 64-127 or functional variants or fragments thereof. A synthetic microRNA or pre-microRNA is a non-naturally occurring or artificial microRNA molecule. According to the invention, the inhibitory RNA or polynucleotide sequence encoding an inhibitory RNA of SEQ ID Nos: 1-17, 64-127 are synthetic microRNAs which are non-naturally occurring, artificial sequences. The invention also provides for functional variants or fragments thereof and as described in the “Polynucleotides” section.

In a further embodiment of the invention, the synthetic microRNAs or synthetic pre-microRNAs are endogenous modified microRNAs. An endogenous modified microRNA molecule refers to a molecule in which the native microRNA molecule originates within the cell and the modification of the microRNA molecule takes place within the cell. Such techniques employed to modify the endogenous microRNA include, but are not limited to, CRISPR and adenosine deaminases acting on RNA (ADARs).

In another embodiment of the invention, there is provided a synthetic microRNA adapted to target a messenger RNA transcribed from a gene, preferably the B2M gene. Suitably, the synthetic microRNA comprises a sequence according to any one of SEQ ID Nos: 1-17, 64-127 or a functional variant or fragment thereof and optionally comprises a sequence according to any one of SEQ ID Nos: 18-34, 128-191 or a functional variant or fragment thereof.

The synthetic RNA according to the present invention are particularly suitable for treatment of chronic inflammatory conditions, particularly ex vivo cell therapy, as they can buffer the augmentation of MHC-I expression in ex vivo therapeutic cells as the cells are exposed to inflammatory stimuli as shown in FIG. 18 and Example 13 below.

### Cells and Cell Therapies

According to the invention, any type of cell may be used. In one embodiment of the invention the cell is a eukaryotic cell, preferably a mammalian cell. The cell can be a stem cell, for example an embryonic stem cells (ESCs), an induced pluripotent stem cells (iPSC) or an adult stem cell.

Thus, in some embodiments, the cell may be any type of stem cell, preferably an induced pluripotent stem cell (iPSC) or a cell derived through differentiation of a stem cell, e.g. a totipotent or pluripotent stem cell, such as an induced pluripotent stem cell. iPSC can be differentiated into substantially any cell type.

In certain particular embodiments, the cell is a differentiated cell including without limitation a dendritic cell, lymphocyte, red blood cell, platelet, hematopoietic cell, pancreatic islet cell, liver cell, muscle cell, keratinocyte, cardiomyocyte, neuronal cell, skeletal muscle cell, ocular cell, mesenchymal cell, fibroblast, lung cell, GI tract cell, vascular cell, endocrine cell and adipocyte. In certain other particular embodiments, the invention provides a method of treating a disease condition in a solid organ.

In some preferred embodiments the cell is an iPSC or a cell derived from an iPSC.

In a preferred embodiment of the invention, the cell is a pancreatic cell. In a preferred embodiment of the invention, the cell is a pancreatic beta cell.

In some preferred embodiments, the cell is a myeloid cell.

In some preferred embodiments the cell is a haematopoietic stem cell (HSC).

In another preferred embodiment of the invention, the cell is an immune cell. Immune cells of interest include neutrophils, eosinophils, basophils, mast cells, monocytes, macrophages, dendritic cells, natural killer cells, and lymphocytes (B cells and T cells). Suitably the cell is a T-cell, preferably a cytotoxic T-cell, e.g. a T-cell adapted for T-cell therapy such as CAR-T cell therapy.

In some embodiments, the cell is a primary T cell, a pan T cell, CD3+ T cell, CD3+ and CD4+ T cell, CD8+ T cell, CD4+T helper cell (optionally a Th1, Th2, Th17, Th9, Tfh or Th22 cell), a memory T cell, CD4+ Treg cell (optionally FOXP3+ Treg cells or a FOXP3− Treg cell).

In some embodiments, the cell is a macrophage, for example an M1 macrophage, an M2 macrophage (e.g. an M2a, M2b, M2c, and M2d macrophage), a Treg macrophage, or a tumour-associated macrophage (TAM).

Cells of the invention may be genetically engineered, e.g. transfected with the inhibitory RNA or polynucleotide sequence encoding an inhibitory RNA, expression cassette, vector, virion or modified miRNA molecule of the invention. Such transfection may take place ex vivo and a composition comprising such transfected cells may then be used in methods disclosed herein.

In an embodiment of the invention there is provided a cell comprising the expression cassette, vector, virion, synthetic microRNA or pre-microRNA of the invention. In another embodiment of the invention, there is provided a cell or cell population obtained from any method of the present invention. In a preferred embodiment, a cell or cell population comprises a modified endogenous miRNA obtained by any of the methods of the invention.

In some embodiments, the cells are a heterozygous knock-in cell line of the inhibitory RNA of interest. In some embodiments, the cells are a homozygous knock-in cell line of the inhibitory RNA of interest.

In another embodiment of the invention there is provided a cell comprising or expressing the synthetic microRNA according to the invention, wherein the cell is modified to express the synthetic microRNA such that the synthetic microRNA targets a messenger RNA, preferably transcribed from the B2M gene. In a more preferred embodiment, the synthetic microRNA degrades the messenger RNA transcribed from the B2M gene, preferably wherein the messenger RNA is partially silenced and B2M protein expression is modified in a range of 1%-90%, optionally 5%-90%, optionally 10%-90%, expression compared to a control cell.

In some preferred embodiments, the cell according to the aspects of the invention shows more silencing, reduction or modification of the functional activity of B2M when the cell is a differentiated progenitor (e.g. iPSC-derived pancreatic cells or iPSC-derived monocytes) compared to when the cell is undifferentiated iPSC. In some embodiments, the functional activity of B2M in the differentiated progenitor is silenced, reduced or modified by at least 10%, 20%, preferably by at last 30%, 40%, 50%, more preferably by at least 60%, 70%, most preferably by at least 80%, 90%, or 95% compared to when the cell is undifferentiated iPSC

In another embodiment, there is provided a cell or composition for use in a method of treating an autoimmune disease or cancer in a subject, wherein the method comprises administering the cell or composition in a therapeutic amount to the subject. The autoimmune disease may be any autoimmune disease, preferably Type I diabetes.

Autoimmune disorders or cancer are typified by chronic inflammation in patients and therefore any cells or cellular compositions transplanted as interventions will be exposed to inflammatory stimuli. The basal and inducible expression of MHC-I on the cell surface can be influenced (e.g. upregulated) by inflammatory stimuli. The cells or compositions for use in a method of treating an autoimmune disease or cancer in a subject according to the present invention are particularly suitable for treatment of chronic inflammatory conditions, particularly ex vivo cell therapy, as the cells or compositions can buffer the augmentation of MHC-I expression in the cells as they are exposed to inflammatory stimuli as shown in FIG. 18 and Example 13 below.

In certain particular embodiments, cells administered to the recipient may or may not be incorporated into an organ in need of such therapy. In certain embodiments, the cells of the invention are differentiated into the desired cell type, either before or after transplantation, and provide the necessary cellular function without itself being incorporated into the tissue at the site of transplantation. For example, in certain embodiments for treating diabetes, the cells of the invention, either as pluripotent stem cells or differentiated pancreatic beta islet cells, are transplanted to a diabetic patient. In some preferred embodiments, for treating diabetes, particularly type I diabetes, the cells of the invention are transplanted into a diabetic patient as differentiated pancreatic beta islet cells (e.g. differentiated from stem cells). The transplanted cells need not reconstitute a functioning pancreas: they just need to secrete insulin in response to glucose levels. In certain particular embodiments, the cells are transplanted into an ectopic location and are not fully incorporated into the pancreas. Transplantation of pluripotent cells of the invention, differentiated cells of the invention, or a tissue differentiated and developed ex vivo from the cells of the invention are all contemplated by the invention. In certain preferred embodiments, the cell is a human cell and the patient is a human patient.

The invention also provides for a method of treating an autoimmune disease or cancer in a subject, preferably wherein the autoimmune disease is Type I diabetes, wherein the method comprises administering the cell or composition in a therapeutic amount to the subject. Further, the invention provides for use of a cell composition according to the invention in the manufacture of a medicament for treating an autoimmune disease or cancer in a subject, preferably wherein the autoimmune disease is Type I diabetes.

In a further embodiment of the invention, there is provided a cell comprising or expressing a sequence encoding a synthetic inhibitory RNA, preferably a synthetic microRNA, wherein the synthetic inhibitory RNA is adapted to target a gene, preferably the B2M gene. In a preferred embodiment of the invention, the sequence encoding the synthetic inhibitory RNA, preferably a synthetic microRNA, encodes one or more of SEQ ID Nos: 1-17, 64-127. In an alternative preferred embodiment, the sequence encoding the synthetic microRNA encodes one or more of SEQ ID Nos: 18-34, 128-191. In yet another alternative preferred embodiment, the sequence encoding the synthetic microRNA comprises one or more of SEQ ID Nos: 35-51.

In an embodiment of the invention, the sequence encoding the synthetic inhibitory RNA, preferably microRNA, is genomic or episomal. The term genomic refers to the sequence being inserted into the genome by, for example, HDR. A genomic insertion can involve a single insertion or a plurality of insertions (e.g. 2, 3, 4, or more). “Insertion” in this context also includes the modification of an endogenous miRNA encoding sequence such that it encodes the relevant inhibitory RNA, as will be discussed below. The term episomal refers to the sequence being introduced into the cell by, for example, vector transduction and thus the sequence is not integrated into the genome within the cell, preferably where the sequence is stably maintained in the cell. In a preferred embodiment, the sequence encoding the synthetic inhibitory RNA is genomic.

In some embodiments of the invention the cell comprises a modified genome in which an endogenous miRNA has been modified such that it is redirected to target B2M. In some embodiments the genome modification can be homozygous, heterozygous or hemizygous. In some other embodiments, the cell comprises a modified genome in which an expression cassette is inserted such that the inhibitory RNA molecule is expressed in the cell. Thus, the present invention contemplates both the redirection of an endogenous inhibitory RNA (especially a miRNA) of a cell to target B2M, and the insertion of a suitable expression construct in the genome to express an inhibitory RNA (especially a miRNA) to target B2M. In the case of an insertion, the inhibitory RNA (especially a miRNA) can be a modified form of an inhibitory RNA (especially a miRNA) that is found in the cell.

Accordingly, in some embodiments of the invention, the sequence encoding the synthetic microRNA is a genomic sequence encoding a modified endogenous microRNA. Suitably the cell is a human cell and the modified genomic sequence encodes a modified form of one of the following endogenous microRNAs:
(i) hsa-mir-191, optionally wherein hsa-mir-191 has been modified to comprise SEQ ID NO: 1, SEQ ID NO: 7 or SEQ ID NO: 100;
(ii) hsa-mir-302a, optionally wherein hsa-mir-302a has been modified to comprise SEQ ID NO: 2, SEQ ID NO: 11, SEQ ID NO: 97, SEQ ID NO: 103, SEQ ID NO: 104 or SEQ ID NO: 109;
(iii) hsa-mir-302c, optionally wherein hsa-mir-302c has been modified to comprise SEQ ID NO: 3, SEQ ID NO: 12, SEQ ID NO: 98, SEQ ID NO: 107, SEQ ID NO: 108 or SEQ ID NO: 111;
(iv) hsa-mir-93, optionally wherein hsa-mir-93 has been modified to comprise SEQ ID NO: 4 or 16;
(v) hsa-mir-106a, optionally wherein hsa-mir-106a has been modified to comprise SEQ ID NO: 5 or SEQ ID NO: 67;
(vi) hsa-mir-106b, optionally wherein hsa-mir-106b has been modified to comprise SEQ ID NO: 6;
(vii) hsa-mir-200c, optionally wherein hsa-mir-200c has been modified to comprise SEQ ID NO: 8;
(viii) hsa-mir-20a, optionally wherein hsa-mir-20a has been modified to comprise SEQ ID NO: 9;
(iv) hsa-mir-21, optionally wherein hsa-mir-21 has been modified to comprise SEQ ID NO: 10;
(x) hsa-mir-363, optionally wherein hsa-mir-363 has been modified to comprise SEQ ID NO: 13;
(xi) hsa-mir-518b, optionally wherein hsa-mir-518b has been modified to comprise SEQ ID NO: 14 or SEQ ID NO: 76;
(xii) hsa-mir-744, optionally wherein hsa-mir-744 has been modified to comprise SEQ ID NO: 15;
(xiii) hsa-mir-99b, optionally wherein hsa-mir-99b has been modified to comprise SEQ ID NO: 17;
(xiv) hsa-mir-320a, optionally wherein hsa-mir-320a has been modified to comprise SEQ ID NO: 64, SEQ ID NO: 73 or SEQ ID NO: 89;
(xv) hsa-mir-520f, optionally wherein hsa-mir-520f has been modified to comprise SEQ ID NO: 65, SEQ ID NO: 77 or SEQ ID NO: 94;
(xvi) hsa-mir-652, optionally wherein hsa-mir-652 has been modified to comprise SEQ ID NO: 66, SEQ ID NO: 78 or SEQ ID NO: 95;
(xvii) hsa-mir-1180, optionally wherein hsa-mir-1180 has been modified to comprise SEQ ID NO: 68 or SEQ ID NO: 79;
(xviii) hsa-mir-15b, optionally wherein hsa-mir-15b has been modified to comprise SEQ ID NO: 69 or SEQ ID NO: 81;
(xix) hsa-mir-182, optionally wherein hsa-mir-182 has been modified to comprise SEQ ID NO: 70, SEQ ID NO: 82 or SEQ ID NO: 99;
(xx) hsa-mir-23a, optionally wherein hsa-mir-23a has been modified to comprise SEQ ID NO: 71 or SEQ ID NO: 87;
(xxi) hsa-mir-26b, optionally wherein hsa-mir-26b has been modified to comprise SEQ ID NO: 72;
(xxii) hsa-mir-335, optionally wherein hsa-mir-335 has been modified to comprise SEQ ID NO: 74 or SEQ ID NO: 90;
(xxiii) hsa-mir-361, optionally wherein hsa-mir-361 has been modified to comprise SEQ ID NO: 75 or SEQ ID NO: 91;
(xxiv) hsa-mir-1307, optionally wherein hsa-mir-1307 has been modified to comprise SEQ ID NO: 80;
(xxv) hsa-mir-205, optionally wherein hsa-mir-205 has been modified to comprise SEQ ID NO: 83;
(xxvi) hsa-mir-22, optionally wherein hsa-mir-22 has been modified to comprise SEQ ID NO: 84;
(xxvii) hsa-mir-221, optionally wherein hsa-mir-221 has been modified to comprise SEQ ID NO: 85;
(xxviii) hsa-mir-222, optionally wherein hsa-mir-222 has been modified to comprise SEQ ID NO: 86;
(xxix) hsa-mir-30e, optionally wherein hsa-mir-30e has been modified to comprise SEQ ID NO: 88;
(xxx) hsa-mir-423, optionally wherein hsa-mir-423 has been modified to comprise SEQ ID NO: 92;
(xxxi) hsa-mir-519c, optionally wherein hsa-mir-519c has ben modified to comprise SEQ ID NO: 93;
(xxxii) hsa-mir-92b, optionally wherein hsa-mir-92b has been modified to comprise SEQ ID NO: 96;
(xxxiii) hsa-mir-30, optionally wherein hsa-mir-30 has been modified to comprise SEQ ID NO: 101 or SEQ ID NO: 102;
(xxxiv) hsa-mir-302b, optionally wherein hsa-mir-302b has been modified to comprise SEQ ID NO: 105, SEQ ID NO: 106 or SEQ ID NO: 110
(xxxv) hsa-mir-375, optionally wherein hsa-mir-375 has been modified to comprise SEQ ID NO: 112, SEQ ID NO: 113 or SEQ ID NO: 114;
(xxxvi) hsa-let-7b, optionally wherein hsa-let-7b has been modified to comprise SEQ ID NO: 115, SEQ ID NO: 119, SEQ ID NO: 122 or SEQ ID NO: 125;
(xxxvii) hsa-let-7g, optionally wherein hsa-let-7g has been modified to comprise SEQ ID NO: 116, SEQ ID NO: 120, SEQ ID NO: 123 or SEQ ID NO: 126; and
(xxxviii) hsa-mir-98, optionally wherein hsa-mir-98 has been modified to comprise SEQ ID NO: 117, SEQ ID NO: 118, SEQ ID NO: 121, SEQ ID NO: 124 or SEQ ID NO: 127.

In a preferred embodiment of the invention, the cell exhibits reduced protein expression, preferably B2M expression. Reduced protein expression means that the level of protein expression is less than the expression seen under normal conditions. Preferably, reduced protein expression means that the protein is not completely eliminated. In a preferred embodiment of the invention, B2M protein expression is not completed eliminated, preferably B2M is expressed at a level of from 1%-90%, optionally 5%-90%, optionally 10%-90%, compared to a control cell. In a preferred embodiment, B2M is expressed at a level of 30%-95% compared to a control cell. In a preferred embodiment, B2M is expressed at a level of 65%-95% compared to a control cell. In a preferred embodiment, B2M is expressed at a level of 65%-89% compared to a control cell. In some embodiments, the control cell is the control parental line (unedited). In some embodiments, the control cell is a cell in which the endogenous miRNA is deleted or disrupted at one or both alleles.

In the cells of the invention, the expression of the protein (preferably B2M) may be modified, degraded, reduced or silenced according to the present invention. In one embodiment of any aspect of the invention, the expression of the protein is modified within a range of 1%-90%, optionally 5%-90%, optionally 10%-90%. Suitably, for example the expression of the protein is within a range of 1%-80%, 1%-70%, 1%-60%, 1%-50%, 1%-40%, 1%-30%, 1%-20%, 1%-10%, 1%-5%, 5%-80%, 5%-70%, 5%-60%, 5%-50%, 5%-40%, 5%-30%, 5%-20%, 5%-10%, 10%-80%, 10%-70%, 10%-60%, 10%-50%, 10%-40%, 10%-30%, 10%-20%, 20%-90%, 30%-90%, 40%-90%, 50%-90%, 10%-20%, 20%-30%, 30%-40%, 40%-50%, 50%-60%, 60%-70%, 70%-80%, or 80%-90% compared to a control cell. It is an advantage of the present invention that the skilled person can modulate, typically reduce, expression of a protein (e.g. B2M) by a desired amount, e.g. to tune expression to a suitable level compared to a control cell. In some embodiments the expression level of B2M or any other cell surface protein in a cell is determined by the amount of B2M expressed on the surface of cell. The amount of B2M present on the surface of cell can be determined by various conventional techniques, e.g. by flow cytometry.

In a preferred embodiment of the invention, the cell exhibits reduced B2M protein expression. In a preferred embodiment of the invention, the cell exhibits reduced B2M protein expression which leads to reduced HLA and/or MHC-I protein expression on the cell surface.

In another preferred embodiment of the invention, the cell is de-targeted from the innate and adaptive immune systems such that an immune response is not mounted against the cell.

In some preferred embodiments, the cell evades the innate immune system. In some preferred embodiments, the cell evades Natural Killer cell mediated immune system activation. In some preferred embodiments, the cell is subjected to lower Natural Killer cell mediated cytotoxicity compared to a control cell (such as a parental cell line (unedited) or a B2M KO cell line (in one or both alleles), or a cancer cell line). In some preferred embodiments, specific lysis by Natural Killer cells is reduced for the cell compared to a control cell (such as a parental cell line (unedited) or a B2M KO cell line (in one or both alleles) or a cancer cell line). In some preferred embodiments, the cell expresses lower levels of B2M protein (and thus lower levels of MHC-I protein cell surface expression) compared to a control cell (e.g. a parental cell line (unedited) or a cell in which the endogenous miRNA is deleted or disrupted at one or both alleles). Partial knockdown of B2M (partial silencing of MHC-I) has been shown to help the cell evade the innate (NK-mediated) immune system (reduced NK-specific lysis).

In a further embodiment of the invention, there is provided a composition comprising the cell or cell population of the invention, which is suitably an induced pluripotent stem cell or a cell derived through differentiation of an induced pluripotent stem cell (e.g. a pancreatic β cell). Such a composition may be a sterile composition suitable for parenteral administration.

There is also provided an inhibitory RNA or polynucleotide encoding an inhibitory RNA, an expression cassette, a vector, a virion, a synthetic microRNA or pre-microRNA, a cell, suitably an induced pluripotent stem cell or cell derived therefrom, or a composition for use in therapy.

In another embodiment of the invention, there is provided an inhibitory RNA or polynucleotide encoding an inhibitory RNA, an expression cassette, a vector, a virion, a synthetic microRNA or pre-microRNA, a cell, suitably an induced pluripotent stem cell, or a composition for use in a method of treating an autoimmune disease or cancer in a subject, wherein the autoimmune disease is preferably Type I diabetes.

In a further embodiment of the invention, there is provided an inhibitory RNA or polynucleotide encoding an inhibitory RNA, an expression cassette, a vector, a virion, a synthetic microRNA or pre-microRNA, for use in a method of degrading the messenger RNA transcribed from the B2M gene in a cell, preferably wherein the messenger RNA is partially silenced and B2M protein expression is modified in a range of 1%-90%, optionally 5%-90%, optionally 10%-90%, expression compared to a control cell. Suitably, the cell is subsequently transferred into a mammal, preferably a human, to treat an autoimmune disease or cancer. The autoimmune disease may be any autoimmune disease, preferably Type I diabetes.

The cells of the invention can be differentiated into various cell types of all three lineages, including without limitation hematopoietic, mesenchymal, pancreatic endoderm, cardiac and keratinocytes cells. In general, each cell type can be analyzed reactivity with human T cells and NK cells, appropriate differentiation markers, and xenotransplantation in immunodeficient mice to examine in vivo developmental potential. A brief discussion of each differentiated cell type follows.

In certain embodiments, the cells of the invention can be differentiated to hematopoietic stem cells, or blood cell types derived from hematopoietic stem cells for treating various hematopoietic diseases currently treated by bone marrow transplantation. Patients receiving transfusion can become refractory to platelet transfusions due to HLA mismatches. Cytopenic patients (neutropenia, thrombocytopenia) can be treated by delivering the cells of the invention-derived, platelets or neutrophils to treat bleeding or infection.

Further, stem cells of the invention-derived dendritic cells are antigen-presenting cells that can be used as cellular vaccines when properly engineered. Donor-derived dendritic cells though are recognised as foreign by the direct pathway; recipient T cells recognise intact donor MHC antigens on the donor cells. In certain embodiments, the dendritic cells of the invention are engineered to express a single chain fusion HLA class I protein and a unique peptide antigen and are used to vaccinate against specific pathogen or tumour antigens.

To obtain hematopoietic cells, pluripotent cells are first allowed to form embryoid bodies, thereafter non-adherent cells were cultured in the presence of hematopoietic cytokines to develop into specific cell lineages. The differentiation of hematopoietic cells can be analyzed by flow cytometry and colony assays. The different cell populations are sorted based on their surface markers, and used to monitor the expression of HLA genes and reactivity with human NK cells and T cells as measured by Elispot, mixed lymphocyte reactions, and cytotoxicity assays. The effectiveness of suppression of NK cell-mediated killing can be examined at different stages of differentiation and transplantation. See Bix et al., 1991, Nature 349, 329-331. The hematopoietic stem cells can also be assayed using xenotransplantation models in, for example, immunodeficient mice (SCID-repopulating cells or SRCs). [00073] The cells of the invention can be differentiated into hematopoietic cell either before or after the cells are administered to a patient. In certain preferred embodiments, the cell is a human cell and the patient is a human. In vitro hematopoietic differentiation can be performed according to established protocols. See for example, Slukvin et al., 2006, J Immunol 776:2924-32, and Chang et al, 2006, Blood 108: 1515-23.

In certain other embodiments, pluripotent cells of the invention can be differentiated into mesenchymal stem cells. MSCs have the potential to form several differentiated cell types, including marrow stromal cells, adipocytes, osteoblasts, and chondrocytes. Thus, inducing pluripotent stem cells to form MSCs (iMSCs) is useful in treating skeletal and joint conditions. The iMSCs can be further differentiated into osteoblasts and formed bone in vivo. Deyle et al., 2012, Mol Ther. 20(1):204-13. Cellular responses of T cells and NK cells to ESCs, iMSCs, and their more terminally differentiated derivatives such as osteoblasts can be examined. In certain particular embodiments, the mesenchymal stem cells are capable of differentiating into non-limiting examples of cell types such as marrow stromal cells, adipocytes, osteoblasts, osteocytes and chondrocytes. The cells of the invention are differentiated into mesenchymal stem cells either before or after the cells are administered to a patient. In certain preferred embodiments, the cell is a human cell and the patient is a human. In vitro mesenchymal differentiation can be performed according to established protocols. See for example, Deyle et al.

In yet other particular embodiments, pluripotent cells can be differentiated into insulin-producing pancreatic islet cells. The cells of the invention can be used to treat insulin-dependent diabetes mellitus. Advantageously, the transplanted cells do not need to reconstitute a functioning pancreas. Rather, they just need to secrete insulin in response to glucose levels. Therefore, the treatment can succeed with different cell doses, with cells that are not perfectly differentiated into adult cell types, and when cells are transplanted into an ectopic location. Specific auto-antigens such as those derived from GAD65 or Insulin can cause autoimmune destruction of β cells in diabetes (Di Lorenzo et al., 2007, Clin Exp Immunol 148, 1-16). The cells of the invention can be differentiated into pancreatic cells as described previously, which employs exposure of cells to different cytokines and drugs to promote sequential formation of mesendoderm, definitive endoderm, and pancreatic progenitors (Kroon et ah, 2008, Nat Biotechnol 26, 443-452). These cells can be further cultured in implants in immunodeficient mice. The cells of the invention can be analyzed at different developmental stages for their reactivity with T cells and NK cells. The cells of the invention are differentiated into pancreatic islet cell either before or after patient administration. In certain preferred embodiments, the cell is a human cell and the patient is a human.

In certain other particular embodiments, cells can be differentiated into cardiomyocytes. The common clinical problems of myocardial infarction and congestive heart failure can be treated by transplanting healthy cardiomyocytes that engraft and re-establish functional myocardium.

The cardiomyocytes of the present invention allow these treatments to proceed with pre-packaged cells and avoid the immunosuppression currently required for allogeneic heart transplants. Physiologically relevant tests can be performed on the cardiomyocytes derived from the cells of the invention, such as electrical conduction and contraction studies. The cells of the invention can be differentiated into cardiomyocytes either before or after the cells are administered to a patient. In certain embodiments, the cells of the invention are differentiated into cardiomyocytes for treating diseases including without limitation myocardial infarction and congestive heart failure. In vitro cardiomyocyte differentiation can be performed according to established protocols. See for example, Lafiamme et al, 2007, Nat Biotechnol 25, 1015-1024.

In yet other particular embodiments, pluripotent cells can be differentiated into keratinocytes. Severe burns and genetic skin conditions require treatment with skin grafts, and this is currently done with a variety of cell sources such as porcine skin grafts and cultured autologous human keratinocytes. Keratinocytes of the invention can provide a major clinical advance, since burns could be treated as an emergency with pre-packaged cells, and genetic diseases such as epidermolysis bullosum can be treated with “normal” cells that do not require correction of the responsible genetic mutations. In many cases the cells only need to engraft long enough for neighbouring host cells to repopulate the affected area. For in vivo differentiation, the cells of the invention can be embedded in polyvinyl alcohol sponge (PVA)-collagen gel implants for transplantation into a recipient. The cells of the invention can be differentiated into keratinocytes either before or after transplantation. In certain preferred embodiments, the cell is a human cell and the patient is a human.

Other cell types or therapeutic approaches will be apparent to the skilled person.

### Compositions

The cells or cell populations of the present invention may be formulated for delivery in a composition. Compositions of the invention suitably comprise an cells or cell populations of the invention. The composition may be provided in the form of a kit.

Thus, in an embodiment of the invention, there is provided a composition comprising a cell or cell population of the invention. Compositions of the invention may also contain other compounds, which may be biologically active or inactive. Suitably, the composition of the invention is a sterile composition suitable for parenteral administration.

While many acceptable carriers known to those of ordinary skill in the art may be employed in the compositions of the invention, the optimal type of carrier used will vary depending on the mode of administration. Compositions of the present invention may be formulated for any appropriate manner of administration, including for example, parenteral, topical, oral, nasal, intravenous, intracranial, intraperitoneal, subcutaneous or intramuscular administration, preferably parenteral e.g., intramuscular, subcutaneous or intravenous administration. For parenteral administration, the carrier preferably comprises water and may contain buffers for pH control, stabilising agents e.g., surfactants and amino acids and tonicity modifying agents e.g., salts and sugars. If the composition is intended to be provided in lyophilised form for dilution at the point of use, the formulation may contain a lyoprotectant e.g., sugars such as trehalose. For oral administration, any of the above carriers or a solid carrier, such as mannitol, lactose, starch, magnesium stearate, sodium saccharine, talcum, cellulose, glucose, sucrose, and magnesium carbonate, may be employed.

Thus, compositions of the invention may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, solutes that render the formulation isotonic, hypotonic or weakly hypertonic with the blood of a recipient, suspending agents, thickening agents and/or preservatives. Alternatively, compositions of the invention may be formulated as a lyophilizate.

The effective amount of the isolated cells of the invention for transplantation or for treating a disease condition depends on a number of factors, such as the type of tissue, the severity of the disease condition, the transplantation reaction, the reason for transplantation, and the age and general health of the patient. The effective amount can be determined by a skilled researcher or clinician by routine practice. Due to the reduced immunogenicity of the transplanted cells, relative large amount of cells can be tolerated by a patient to achieve the desired therapeutic effects. Alternatively, the cells can be repeatedly transplanted at intervals until a desired therapeutic effect is achieved.

The route for administration of the cells of the invention is not limited in any particular way. Exemplary delivery routes include without limitation intravenous, intramuscular, subdermal, intraperitoneal, transcutaneous, intracutaneous, and subcutaneous route. The cells of the present invention can also be administered topically by injection. For example, the cells can be injected into an injured joint, a fractured bone, an infarct site, an ischemic site or their periphery.

In certain particular embodiments, the cells are administered via a delivery device including without limitation a syringe. For example, the cells can be suspended in a solution or a pharmaceutical composition contained in such a delivery device. The “solution” or “pharmaceutical composition” comprises a physiological compatible buffer and optionally a pharmaceutically acceptable carrier or diluent in which the cells of the invention remain viable. The use of such carriers and diluents is well known in the art. The solution includes without limitation physiologically compatible buffers such as Hank's solution, Ringer' solution, or physiologically buffered saline. The cells can be kept in the solution or pharmaceutical composition for short term storage without losing viability. In certain particular embodiments, the cells are frozen for long term storage without losing viability according to cryopreservation methods well-known in the art.

Aqueous injection suspensions may contain substances which increase the viscosity of the suspension, such as sodium carboxymethyl cellulose, sorbitol, or dextran, but still fluid to the extent that can be easily delivered by syringe injection. The solution is preferably sterile, stable under the conditions of manufacture and storage and is free of microorganism contamination through the use of, for example, parabens, chlorobutanol, phenol, ascorbic acid, thimerosal, and the like. The cells contained in the solution can be stem cells or differentiated cells as described herein, in a pharmaceutically acceptable carrier or diluent and, as required, other ingredients indicated above.

The cells may be administered systemically (e.g., intravenously) or locally (e.g., directly into a myocardial defect under the guidance of echocardiogram, or by direct application to damaged tissues or organs accessible during open surgery). For injections, the cells may be in an injectable liquid suspension preparation or in a biocompatible medium which is injectable in liquid form and becomes semi-solid at the site of damaged tissue. A syringe, a controllable endoscopic delivery device or other similar devises can be used so long as the needle lumen is of sufficient diameter (e.g. at least 30 gauge or larger) to avoid physical damages to the cells during delivery.

In certain other embodiments, the cells can be transplanted via a solid support, e.g., a planar surface or three-dimensional matrix. The matrix or planar surface is surgically implanted into the appropriate site in a patient. For example, a patient needing a pancreatic graft can have differentiated cells on a solid support surgically implanted in the peritoneum or the portal vein of the liver. Exemplary solid support includes without limitation a patch, a gel matrix (such as GELFOAM® from Pharmacia-Upjohn), polyvinyl alcohol sponge (PVA) collagen gel implants (such as IVALON, Unipoint Industries, High Point, NC) and other similar or equivalent devices. A variety of other encapsulation technologies can be used with the cells of the invention, for example, WO 91/10470; WO 91/10425; U.S. Pat. Nos. 5,837,234; 5,011,472; 4,892,538).

### Methods

In an embodiment of the present invention, there is provided a method of modifying the expression of a protein, suitably B2M, in a cell. There is further provided a method of modifying the translation of an RNA transcript derived from a protein coding gene, suitably B2M, in a cell. Suitably, the methods comprise introducing into a cell an inhibitory RNA or polynucleotide encoding an inhibitory RNA, an expression cassette, a vector, a virion, or a synthetic microRNA or pre-microRNA of the invention. Introduction into the cell may be by any suitable method, for example transfection.

In another embodiment of the invention, there is provided a method of modifying the translation of a messenger RNA transcript derived from a protein coding gene and subsequent expression of a protein in a cell comprising introducing into the cell an inhibitory RNA or polynucleotide encoding an inhibitory RNA an expression cassette, a vector, a virion or a synthetic microRNA or pre-microRNA of the invention.

In yet another embodiment of the invention, there is provided a method of modifying (preferably reducing) the translation of an messenger RNA transcript derived from a protein coding gene (preferably the B2M gene) and subsequent expression of a protein (preferably B2M) in a cell comprising introducing into the cell a DNA editing agent conferring a silencing specificity of a microRNA towards a target RNA of interest, wherein the microRNA is modified such that the sequence of the microRNA comprises any one of SEQ ID Nos: 1-17, 64-127 (or a variant or fragment thereof as discussed above), thereby modifying the translation of the messenger RNA transcript into a protein molecule.

In other aspects of the present invention, the method uses a gene editing technology utilising inhibitory RNA molecules designed to target and interfere with an RNA molecule of interest (endogenous or exogenous to the eukaryotic cell, preferably endogenous to the eukaryotic cell).

The gene editing technology of some embodiments of the invention comprises genome editing of an inhibitory RNA molecule (e.g. endogenous) yet it is stable and heritable. The present inventors use a Genome Editing induced Gene Silencing (GEiGS) platform capable of utilising a eukaryotic cell's endogenous non-coding RNA molecules including e.g. RNA silencing molecules (e.g. siRNA, miRNA, piRNA, tasiRNA, tRNA, rRNA, antisense RNA, etc.) and modifying them to target any RNA target of interest. Using GEiGS, the skilled person is able to screen potential non-coding RNA molecules, editing a few nucleotides in these endogenous RNA molecules, and thereby redirecting their activity and/or specificity to effectively and specifically target any RNA of interest including, for instance, mRNA encoding B2M protein.

Accordingly, there is provided a method of modifying the translation of an RNA transcript derived from a protein coding gene, suitably B2M, in a cell comprising introducing into the cell an RNA editing agent conferring a silencing specificity of a non-coding RNA molecule towards a target RNA of interest, wherein the non-coding RNA molecule is modified such that the sequence of the non-coding RNA molecule is that of any one of SEQ ID Nos: 1-17, 64-127, thereby modifying the translation of the RNA transcript into a protein molecule.

According to a specific embodiment of the invention, the cell is a eukaryotic cell, preferably a mammalian cell. According to another specific embodiment of the invention, the cell is an induced pluripotent stem cell or a cell derived through differentiation of an induced pluripotent stem cell. Preferably, the cell is a pancreatic beta cell. Alternatively, it may be preferred that the cell is an immune cell.

In one embodiment, the cell is a human cell and the method comprises editing the sequence of an endogenous micro RNA selected from the group consisting of: hsa-mir-191; hsa-mir-302a; hsa-mir-302c; hsa-mir-93; hsa-mir-106a; hsa-mir-106b; hsa-mir-200c; hsa-mir-20a; hsa-mir-21; hsa-mir-363; hsa-mir-518b; hsa-mir-744; hsa-mir-99b; hsa-mir-320a; hsa-mir-520f; hsa-mir-652; hsa-mir-1180; hsa-mir-15b; hsa-mir-182; hsa-mir-23a; hsa-mir-26b; hsa-mir-335; hsa-mir-361; hsa-mir-1307; hsa-mir-205; hsa-mir-22; hsa-mir-221; hsa-mir-222; hsa-mir-30e; hsa-mir-423; hsa-mir-519c; hsa-mir-92b; hsa-mir-30; hsa-mir-302b; hsa-mir-375; hsa-let-7b; hsa-let-7g; and hsa-mir-98.

In an embodiment of the invention, the protein is B2M or the messenger RNA transcript derived from a protein coding gene encodes B2M protein. In another embodiment of the invention the expression of the protein in the cell is modified in a range of 1%-90%, optionally 5%-90%, optionally 10%-90%, when compared to a control cell.

The expression of the protein (preferably B2M) may be modified, degraded, reduced or silenced according to the present invention. In one embodiment of any aspect of the invention, the expression of the protein is modified within a range of 1%-90%, optionally 5%-90%, optionally 10%-90%. Suitably, for example, the expression of the protein is within a range of 1%-80%, 1%-70%, 1%-60%, 1%-50%, 1%-40%, 1%-30%, 1%-20%, 1%-10%, 1%-5%, 5%-80%, 5%-70%, 5%-60%, 5%-50%, 5%-40%, 5%-30%, 5%-20%, 5%-10%, 10%-80%, 10%-70%, 10%-60%, 10%-50%, 10%-40%, 10%-30%, 10%-20%, 20%-90%, 30%-90%, 40%-90%, 50%-90%, 10%-20%, 20%-30%, 30%-40%, 40%-50%, 50%-60%, 60%-70%, 70%-80%, or 80%-90% compared to a control cell. It is an advantage of the present invention that the skilled person can modulate, typically reduce, expression of a protein by a desired amount, e.g. to a suitable level compared to a control cell. In some embodiments the expression level of B2M or any other cell surface protein in a cell is determined by the amount of B2M expressed on the surface of cell. The amount of B2M present on the surface of cell can be determined by various conventional techniques, e.g. by flow cytometry.

Various non-limiting examples of genome editing methods and RNA editing agents used to introduce nucleic acid alterations in inhibitory RNA molecules can be used according to specific embodiments of the invention.

Genome Editing using engineered endonucleases—this approach refers to a reverse genetics method using artificially engineered nucleases to typically cut and create specific double-stranded breaks (DSBs) at a desired location(s) in the genome, which are then repaired by cellular endogenous processes such as, homologous recombination (HR) or non-homologous end-joining (NHEJ). NHEJ directly joins the DNA ends in a double-stranded break (DSB) with or without minimal ends trimming, while HR utilizes a homologous donor sequence as a template (i.e. the sister chromatid formed during S-phase) for regenerating/copying the missing DNA sequence at the break site. In order to introduce specific nucleotide modifications to the genomic DNA, a donor DNA repair template containing the desired sequence must be present during HR (exogenously provided single stranded or double stranded DNA).

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks (DSBs), several distinct classes of nucleases have been discovered and bioengineered to date. These include the meganucleases, Zinc finger nucleases (ZFNs), transcription-activator like effector nucleases (TALENs) and CRISPR/Cas9 system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks (DSBs) in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (DSBs).

ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break (DSB).

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break (DSB). Repair of these double-stranded breaks (DSBs) through the non-homologous end-joining (NHEJ) pathway often results in small deletions or small sequence insertions (Indels). Since each repair made by NHEJ is unique, the use of a single nuclease pair can produce an allelic series with a range of different insertions or deletions at the target site.

In general NHEJ is relatively accurate (about 75-85% of DSBs in human cells are repaired by NHEJ within about 30 min from detection) in gene editing erroneous NHEJ is relied upon as when the repair is accurate the nuclease will keep cutting until the repair product is mutagenic and the recognition/cut site/P AM motif is gone/mutated or that the transiently introduced nuclease is no longer present. The deletions typically range anywhere from a few base pairs to a few hundred base pairs in length, but larger deletions have been successfully generated in cell culture by using two pairs of nucleases simultaneously. In addition, when a fragment of DNA with homology to the targeted region is introduced in conjunction with the nuclease pair, the double-stranded break (DSB) can be repaired via homologous recombination (HR) (e.g. in the presence of a donor template) to generate specific modifications.

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system and all its variants (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form a RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821).

It was further demonstrated that a synthetic chimeric guide RNA (sgRNA) composed of a fusion between crRNA and tracrRNA could direct Cas9 to cleave DNA targets that are complementary to the crRNA in vitro. It was also demonstrated that transient expression of Cas9 in conjunction with synthetic sgRNAs can be used to produce targeted double-stranded breaks (DSBs) in a variety of different species (Cho et ah, 2013; Cong et ah, 2013; DiCarlo et ah, 2013; Hwang et al., 2013a, b; Jinek et ah, 2013; Mali et ah, 2013).

The CRISPR/Cas system for genome editing contains two distinct components: a sgRNA and an endonuclease e.g. Cas9. The sgRNA (also referred to herein as short guide RNA (sgRNA)) is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base pairing between the sgRNA sequence and the complement genomic DNA/RNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break (DSB). The double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks (DSBs) in the genomic DNA or RNA.

A significant advantage of CRISPR/Cas is that the high efficiency of this system is coupled with the ability to easily create synthetic sgRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the sgRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single strand break, or nick, is mostly repaired by single strand break repair mechanism involving proteins such as but not only, PARP (sensor) and XRCC1/LIG Ill complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas9 nickase to create a double-nick by designing two sgRNAs with target sequences in close proximity and on opposite strands of the genomic DNA would decrease off-target effect as either sgRNA alone will result in nicks that are not likely to change the genomic DNA, even though these events are not impossible.

Modified versions of the Cas9 enzyme containing two inactive catalytic domains (dead Cas9, or dCas9) have no nuclease activity while still able to bind to DNA based on sgRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Additional variants of Cas9 which may be used by some embodiments of the invention include, but are not limited to, CasX and Cpf1 (also known as Cas12a). CasX enzymes comprise a distinct family of RNA-guided genome editors which are smaller in size compared to Cas9 and are found in bacteria (which is typically not found in humans), hence, are less likely to provoke the immune system/response in a human. Also, CasX utilizes a different PAM motif compared to Cas9 and therefore can be used to target sequences in which Cas9 PAM motifs are not found.

The CRISPR system may be fused with various effector domains, such as DNA cleavage domains. The DNA cleavage domain can be obtained from any endonuclease or exonuclease. Non-limiting examples of endonucleases from which a DNA cleavage domain can be derived include, but are not limited to, restriction endonucleases and homing endonucleases (see, for example, New England Biolabs Catalog or Belfort et al. (1997) Nucleic Acids Res.). In exemplary embodiments, the cleavage domain of the CRISPR system is a FokI endonuclease domain or a modified FokI endonuclease domain. In addition, the use of Homing Endonucleases (HE) is another alternative. Hes are small proteins (<300 amino acids) found in bacteria, archaea, and in unicellular eukaryotes. A distinguishing characteristic of Hes is that they recognize relatively long sequences (14-40 bp) compared to other site-specific endonucleases such as restriction enzymes (4-8 bp). Hes have been historically categorized by small conserved amino acid motifs. At least five such families have been identified: LAGLIDADG; GIY-YIG; HNH; His-Cys Box and PD-(D/E)xK, which are related to EdxHD enzymes and are considered by some as a separate family. At a structural level, the HNH and His-Cys Box share a common fold (designated bba-metal) as do the PD-(D/E)xK and EdxHD enzymes. The catalytic and DNA recognition strategies for each of the families vary and lend themselves to different degrees to engineering for a variety of applications. Exemplary Homing Endonucleases which may be used according to some embodiments of the invention include, without being limited to, I-CreI, I-TevI, I-HmuI, I-PpoI and I-Ssp68031.

Modified versions of CRISPR, e.g. dead CRISPR (dCRISPR-endonuclease), may also be utilized for CRISPR transcription inhibition (CRISPRi) or CRISPR transcription activation (CRISPRa).

Other versions of CRISPR which may be used according to some embodiments of the invention include genome editing using components from CRISPR systems together with other enzymes to directly install point mutations into cellular DNA or RNA.

Thus, according to one embodiment, the editing agent is an DNA editing agent. Preferably, the DNA editing agent comprises a DNA editing system. More preferably, the DNA editing system comprises a meganuclease, a zinc finger nucleases (ZFN), a transcription-activator like effector nuclease (TALEN), homology directed repair (HDR), CRISPR-endonuclease, dCRISPR-endonuclease, or a homing endonuclease.

According to one embodiment, the DNA editing agent does not comprise an endonuclease. According to another embodiment, the DNA editing agent comprises an endonuclease. Suitably, in some embodiments the endonuclease is any one of Cas9, Cas12a, CasX, CasY, CasPhi, MAD7 or Cas13 or functional variants thereof. According to a preferred embodiment, the endonuclease is Cas9. According to another preferred embodiment, the endonuclease comprises a catalytically inactive endonuclease.

According to yet a further embodiment of the invention, the DNA editing agent is linked to a reporter for monitoring expression in a cell, e.g. a eukaryotic cell. The reporter may be a fluorescent reporter protein. The term “a fluorescent protein” refers to a polypeptide that emits fluorescence and is typically detectable by flow cytometry, microscopy or any fluorescent imaging system, therefore can be used as a basis for selection of cells expressing such a protein. Examples of fluorescent proteins that can be used as reporters are, without being limited to, the Green Fluorescent Protein (GFP), the Blue Fluorescent Protein (BFP) and the red fluorescent proteins (e.g. dsRed, mCherry, RFP). A non-limiting list of fluorescent or other reporters includes proteins detectable by luminescence (e.g. lucif erase) or colorimetric assay (e.g. GUS). According to a specific embodiment, the fluorescent reporter is a red fluorescent protein (e.g. dsRed, mCherry, RFP) or GFP.

Aspects of the invention are demonstrated by the following non-limiting examples.

### EXAMPLES

### Introduction

### Advantages of GEiGS

Since its discovery in the 1990s RNAi has been leveraged in various forms to silence desired target genes, mostly using oligos (siRNA) or ectopically expressed hairpins (shRNA). miRNA-based vectors for shRNA have been used extensively—these are mimics of endogenous miRNAs in which the stem sequence has been swapped for the shRNA sequence of choice. Several scaffolds are used, the most popular being miR-30 (Fellmann et al., 2013). miR30-based vectors are more efficient than standard hairpin constructs, being more effective at silencing the target and having a superior off-target profile, and for this reason they are now used in shRNA clinical applications (Esrick et al., 2021).

In its current form miR-based shRNA is still implemented by ectopic expression, usually as a lentiviral transgene integrated in the genome at random or within safe harbour loci such as AAVS1, where they are being transcribed from exogenous regulatory elements. GEiGS® on the other hand uses modification of endogenous loci encoding silencing molecules to redirect the silencing specificity of the silencing RNAs encoded in these loci, leveraging the program of expression of the endogenous loci, which brings very distinct advantages, namely the ability to induce stable, tuneable and programmable gene silencing.
1. Stability. siRNA oligos are transient in nature, while shRNA suffers from variegated expression due to transgenes being inactivated through epigenetic mechanisms. By contrast, endogenous loci are never unpredictably silenced, so the GeiGS® silencing RNA (sRNA) is stably expressed.
2. Tunability. GEiGS® allows the redirection of miRNA scaffolds with different levels of expression, therefore the abundance of the GEiGS® sRNA can be tuned according to the cell engineering requirements. This is notoriously difficult to achieve through current RNAi approaches and impossible through CRISPR KO which is binary in nature.
3. Programmability. Importantly, GEiGS® can provide programmable gene silencing. By redirecting miRNAs that are developmentally regulated or are cell state-specific, GEiGS® silencing can be programmed to be deployed only when therapeutic cells reach a specific stage in differentiation.
4. Specificity. Importantly in ensuring that the expression level of the redirected miRNA scaffold and therefore the abundance of the GEiGS® sRNA are retained at physiological levels in the cells, the normal biogenesis pathway for sRNA generation is not super-saturated with the consequential benefit for far lower occurrences of off-target gene silencing effects. This is starkly contrasted with shRNA and siRNA approaches which notoriously generate artefactual off target gene effects.

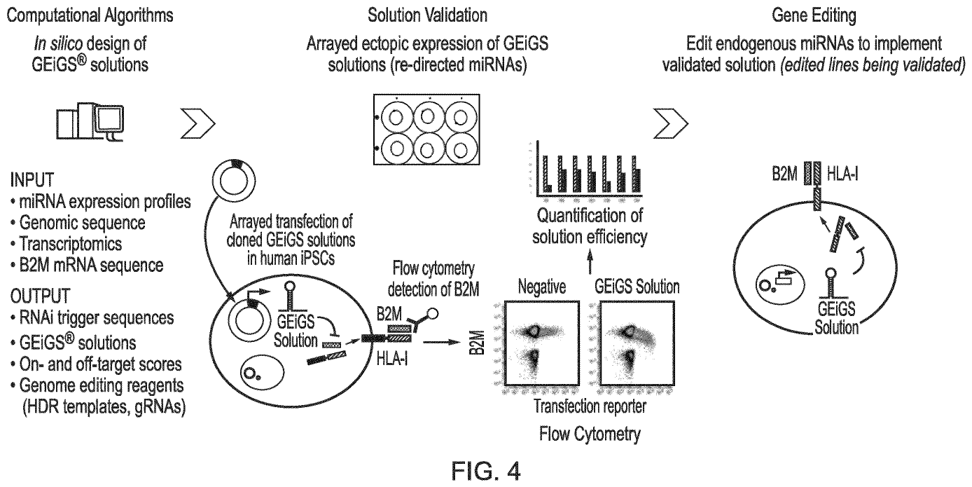

The GEiGS® technology is deployed through the following workflow (see FIG. 4):
1. Computational design of GEiGS® solutions.
2. Experimental validation of the solutions silencing activity through ectopic expression.
3. Gene editing of the endogenous miRNA scaffold in the desired cell type.

GEiGS® solutions validated experimentally can be used in therapeutic applications, for example, either as lentiviral transgenes or edited into the genome to be expressed from an endogenous miRNA locus, to silence for example, B2M in primary human cells. Further information of GEiGS® can be found in WO2019/058253, WO2020/183414 and WO2020/183419.

### Example 1—Computational Design of GEiGS Solutions

B2M solutions were generated using a computational pipeline. Pipeline inputs are: the mRNA sequence of B2M (GenBank Accession: NM_004048.4), the human genome reference sequence (GRCh38), miRNA annotations (miRbase v22) and cell type specific miRNA quantifications.

Expression of miRNAs in human iPSCs and ESCs was quantified using publicly available next-generation sequencing (NGS) datasets: ENCODE accession ENCSR958UOC, ENCSR430YFL.

The computational pipeline run generated a short-list of 50 GEiGS® solutions expected to be able to silence B2M. The GEiGS® solution designs were comprised of a modified sequence of an endogenous pre-miRNA (hairpin)—also referred to as the endogenous miRNA scaffold-modified to encode a novel silencing RNAi trigger instead of its native guide strand. The novel GEiGS® silencing RNA matched the B2M mRNA by sequence complementarity and was predicted to lead to the silencing of B2M through the RNA interference pathway.

The solution design process also generated corresponding negative control constructs—also referred to as ‘dummy’ solutions—in which the same endogenous miRNA scaffold was modified as in the corresponding GEiGS® solution but to express a scrambled sequence of the GEiGS® silencing RNA.

### Example 2—Validation of GEiGS Solution Silencing Activity Through Plasmid-Based Ectopic Expression

19 designs (17 B2M GEiGS solutions and 2 ‘dummy’ controls were used to generate extended GEiGS solutions. Extended solutions were comprised of a GEiGS solution as designed computationally, plus 50-150 bases of genomic sequence flanking on both sides of the endogenous pre-miRNA that the respective GEiGS solution was based on. This was included to avoid omitting important sequences that act as a signal for the enzyme Drosha.

The solutions tested were as follows:

TABLE 1
Sequences of computationally-designed B2M-targeting
RNAi trigger sequences and GEiGS solutions

| GEiGS | | GEiGS mature sRNA | GEiGS solution sequence |
|---|---|---|---|
| Solution | miRNA | sequence (guide) | (pre-miRNA hairpin) |
| ID | scaffold | [SEQ ID NO: #] | [SEQ ID NO: #] |
| Sol-8 | hsa-mir- | TTCAGTGTAGTACA | CGGCTGGACAGCGGGttcagtgtagtacaa |
| | 191 | AGAGATAGA [1] | gagatagaTTGTCTCCAGAGCATTCTCT |
| | | | ATCTGCTGACTACACTGCCCCTGCTC |
| | | | TCCTGCCT [18] |
| Sol-11 | hsa-mir- | TTTCAATTCTCTCTC | CCACAAGATTGGAGAGAGTATTGAAA |
| | 302a | CATTCTTC [2] | CTTTGAAACTAAAGAAGtttcaattctctctcc |
| | | | attcttcTGG [19] |
| Sol-12 | hsa-mir- | TTCAGTAAGTCAAC | CCTACATTTTAGTTGACTTACTGCAG |
| | 302c | TTCAATGT [3] | CTGTGTGAAACAAAAGTttcagtaagtcaa |
| | | | cttcaatgtAGG [20] |
| Sol-19 | hsa-mir- | TTCAGTGTAGTACA | CTGGGGGCTCttcagtgtagtacaagagatag |
| | 93 | AGAGATAGA [4] | aTGTGATTACCCAACTCTTCTCTGTAC |
| | | | TTACACTGCCCGAGCCCCCGG [21] |
| Sol-20 | hsa-mir- | TAAATTTTCCCCCA | CCTTGGCCATGTtaaattttcccccaaattctaa |
| | 106a | AATTCTAAG [5] | gCTTTTTGAGATCTTGAATTAGGGGG |
| | | | AAAATTCTAACATTACCATGG [22] |
| Sol-21 | hsa-mir- | TATATGACAAAATG | CCTGCCGGGGCtatatgacaaaatgtttcattc |
| | 106b | TTTCATTC [6] | GTGGTCCTCTCCGTGCGACCAAACA |
| | | | TTGTGTCATAGCTGCTCCAGCAGG |
| | | | [23] |
| Sol-26 | hsa-mir- | TACTTTATCAAATGT | CGGCTGGACAGCGGGtactttatcaaatgta |
| | 191 | ATAAGAAG [7] | taagaagTTGTCTCCAGAGCATTCCTTC |
| | | | TTAGCCATTGATAAAGCCCCTGCTCT |
| | | | CCTGCCT [24] |
| Sol-27 | hsa-mir- | TAGAATTATAAAGA | CCCACTGACTTCTTAATAATTCTTGG |
| | 200c | AGATCATGT [8] | GTGCGGTTGGGAGTCTCtagaattataaa |
| | | | gaagatcatgtGG [25] |
| Sol-29 | hsa-mir- | TAACAAATTTCCAAT | GTAGCACtaacaaatttccaataatcctgtTGTT |
| | 20a | AATCCTGT [9] | TAGTTATACAGATTATTGGAAATTTGT |
| | | | AAAGTACTGC [26] |
| Sol-30 | hsa-mir- | TGTTAACATTATTAT | TGTCGGGtgttaacattattataaccctacTGTT |
| | 21 | AACCCTAC [10] | GAATCTCATGGAGGGTCATAATATGT |
| | | | TAACATCTGACA [27] |
| Sol-35 | hsa-mir- | TTATATTTCTAAATT | CCACGGGGTAAAATTTAGTAATATAA |
| | 302a | TTCCCCCA [11] | CTTTGAAACTAAAGAAGttatatttctaaatttt |
| | | | cccccaTGG [28] |
| Sol-36 | hsa-mir- | TAACATTATTATAAC | CCTATGTATTGTTATAATAATGTCAGC |
| | 302c | CCTACAT [12] | TGTGTGAAACAAAAGTtaacattattataacc |
| | | | ctacatAGG [29] |
| Sol-41 | hsa-mir- | TATTATAACCCTACA | TGTTGTCAAAATGTACAGTATATAATA |
| | 363 | TTTTGTG [13] | TTGATGAGTATCATAGGAGAAAtattata |
| | | | accctacattttgtgAACC [30] |
| Sol-43 | hsa-mir- | TTAACTTATGCACG | TCATGCTGTGGCAGTTCAGCGTGAAT |
| | 518b | CTTAACTA [14] | AAGTTTCAATTGTCTGAAAGAAAAttaa |
| | | | cttatgcacgcttaactaTTACGGTTTGA [31] |
| Sol-47 | hsa-mir- | TAACTATCTTAACAA | TTGGGCAAGGtaactatcttaacaagctttgaG |
| | 744 | GCTTTGA [15] | TCTTACTGAAGGTTTCCTGGAAACCA |
| | | | CGCACATCAAAGCTGTATAAAATAGA |
| | | | ACCTTACTCGGTC [32] |
| Sol-49 | hsa-mir- | TTACTTTATCAAATG | CTGGGGGCTCttactttatcaaatgtataagaaT |
| | 93 | TATAAGAA [16] | GTGATTACCCAACTTCTATAATTTGTA |
| | | | TAAAGTCCCGAGCCCCCGG [33] |
| Sol-50 | hsa-mir- | TTTATCAAATGTATA | GGCACCtttatcaaatgtataagaagtaGGGC |
| | 99b | AGAAGTA [17] | CTTCGCTACTCACATCCTATTGATTT |
| | | | GATAACCGTGTC [34] |
| Neg Ctrl | hsa-mir- | TTAACTAAACAACA | CCAACCCTttaactaaacaacaacaaacagG |
| 1 | 302a | ACAAACAG [52] | AAACAAAATTGTTTGTTGTGGTTTAGT |
| | | | TGTGGGGATGG [53] |
| Neg Ctrl | hsa-mir- | TTTACGTCGATAAA | CCCGTTCCGATTTAACGACGTAATGG |
| 2 | 200c | TCAGGACAC [54] | GTGCGGTTGGGAGTCTCtttacgtcgataa |
| | | | atcaggacacGG [55] |

The sequences of the extended solutions were as follows [SEQ ID NO: #]:

>hsa-mir-302a_11_B2M-exon2-2
[35]
GCCAGCACACCTTTTGTTACAAAATTTTTTTGTTATTGTGTTTTAAGGTTACTAAGCTTGT
TACAGGTTAAAGGATTCTAACTTTTTCCAAGACTGGGCTCCCCACAAGATTGGAGAGAG
TATTGAAACTTTGAAACTAAAGAAGTTTCAATTCTCTCTCCATTCTTCTGGTAAGTCTTCC
TTTTACATTTTTATTATTTTTTTAGAAAATAACTTTATTGTATTGACCGCAGCTCATATATT
TAAGCTTTATTTTGTATTTTTACATCTGTTA
>hsa-mir-302a_35_B2M-exon4
[36]
GCCAGCACACCTTTTGTTACAAAATTTTTTTGTTATTGTGTTTTAAGGTTACTAAGCTTGT
TACAGGTTAAAGGATTCTAACTTTTTCCAAGACTGGGCTCCCCACGGGGTAAAATTTAG
TAATATAACTTTGAAACTAAAGAAGTTATATTTCTAAATTTTCCCCCATGGTAAGTCTTCC
TTTTACATTTTTATTATTTTTTTAGAAAATAACTTTATTGTATTGACCGCAGCTCATATATT
TAAGCTTTATTTTGTATTTTTACATCTGTTA
>hsa-mir-302c_12_B2M-exon2-2
[37]
TTTACTTCTCCAAAATAGAACACGCTAACCTCATTTGAAGGGATCCCCTACATTTTAGTT
GACTTACTGCAGCTGTGTGAAACAAAAGTTTCAGTAAGTCAACTTCAATGTAGGTGTCT
CCAAGCCAGCACACCTTTTGTTACAAAATTTTTTTGTTATTGTGTTTTAAGGTTACTAAGC
TTGTTACAGGTTAAAGGATTCTAACTTTTTCCAAGA
>hsa-mir-302c_36_B2M-exon4
[38]
TTTACTTCTCCAAAATAGAACACGCTAACCTCATTTGAAGGGATCCCCTATGTATTGTTA
TAATAATGTCAGCTGTGTGAAACAAAAGTTAACATTATTATAACCCTACATAGGTGTCTC
CAAGCCAGCACACCTTTTGTTACAAAATTTTTTTGTTATTGTGTTTTAAGGTTACTAAGCT
TGTTACAGGTTAAAGGATTCTAACTTTTTCCAAGA
>hsa-mir-20a_29_B2M-exon4
[39]
GAATGATTTTTACTAATTTTGTGTACTTTTATTGTGTCGATGTAGAATCTGCCTGGTCTAT
CTGATGTGACAGCTTCTGTAGCACTAACAAATTTCCAATAATCCTGTTGTTTAGTTATAC
AGATTATTGGAAATTTGTAAAGTACTGCTAGCTGTAGAACTCCAGCTTCGGCCTGTCGC
CCAATCAAACTGTCCTGTTA
>hsa-mir-200c_27_B2M-exon4
[40]
AGCAGGGCTCACCAGGAAGTGTCCCCAGGGACTCGGGTGGTGGGGGGATGGGAGCC
AGGGATCTGCAGCTTTTCCGCAGGGATCCTGGGCCTGAAGCTGCCTGACCCAAGGTG
GGCGGGCTGGGCGGGGGCCCACTGACTTCTTAATAATTCTTGGGTGCGGTTGGGAGT
CTCTAGAATTATAAAGAAGATCATGTGGCCCCTGTCCCTGTGTCAGCAACATCCATCGC
CTCAGGTCCCCAGCCCTTAGCTGGCTGCAGCCCCCTCCCCACTTCCCACGCACCCCG
GAAGCCCCTCGTCTTGAGCTGAGAGCGTTGCACAAGGGGTGG
>hsa-mir-21_30_B2M-exon4
[41]
GTTCGATCTTAACAGGCCAGAAATGCCTGGGTTTTTTTGGTTTGTTTTTGTTTTTGTTTTT
TTATCAAATCCTGCCTGACTGTCTGCTTGTTTTGCCTACCATCGTGACATCTCCATGGCT
GTACCACCTTGTCGGGTGTTAACATTATTATAACCCTACTGTTGAATCTCATGGAGGGT
CATAATATGTTAACATCTGACATTTTGGTATCTTTCATCTGACCATCCATATCCAATGTTC
TCATTTAAACATTACCCAGCATCATTGTTTATAATCAGAAACTCTGGTCCTTCTGTCTGG
TGGCACTTAGAGTCTTTTGTGCCATAATGCA
>hsa-mir-363_41_B2M-exon4
[42]
TATCGTCTTATTTTAACTTTTAAAAGCCGTAAGTTCTGATATTTAGTCATTGTAAAATGAT
CTGTTTTGCTGTTGTCAAAATGTACAGTATATAATATTGATGAGTATCATAGGAGAAATAT
TATAACCCTACATTTTGTGAACCGCAGGACCTTTGTTGGCGACATTCCTGATCAGCGCT
ACAGTAAGCTAGATGGTAAAAAATCCTTTCTCTAGTTGCTGCTATTAAAAAATAATTTGAA
ATATATTTGGATTTCTAAAAACTATGATAGCTA
>hsa-mir-93_19_B2M-exon2-2
[43]
AGGCCTTTTCCCCACTTCTTAACCTTCACTGAGAGGGTGGTTGGGGTCTGTTTCACTCC
ATGTGTCCTAGATCCTGTGCTACAGACCTTCCTTTCTGTCCTCCCGTCTTGGACCTCAG
TCCTGGGGGCTCTTCAGTGTAGTACAAGAGATAGATGTGATTACCCAACTCTTCTCTGT
ACTTACACTGCCCGAGCCCCCGGGACACGTTCTCTCTGCCAATTGTCTTCTTGGCTGA
GCTCCCCAAGCTCCATCTGTCATGCTGGGGAGCCCAGTGGCGTTCAAAAGGGTCTGGT
>hsa-mir-93_49_B2M-exon4
[44]
AGGCCTTTTCCCCACTTCTTAACCTTCACTGAGAGGGTGGTTGGGGTCTGTTTCACTCC
ATGTGTCCTAGATCCTGTGCTACAGACCTTCCTTTCTGTCCTCCCGTCTTGGACCTCAG
TCCTGGGGGCTCTTACTTTATCAAATGTATAAGAATGTGATTACCCAACTTCTATAATTT
GTATAAAGTCCCGAGCCCCCGGGACACGTTCTCTCTGCCAATTGTCTTCTTGGCTGAG
CTCCCCAAGCTCCATCTGTCATGCTGGGGAGCCCAGTGGCGTTCAAAAGGGTCTGGT
>hsa-mir-106a_20_B2M-exon4
[45]
TTTACTTGTTTAGTTTCTAATATGTGTGTGTTTGTTTTGTTGTTTTAACCAGGTGAGTCTG
CATGGATCTGTGAGGACGGAAAAGAAGAGCTCCTGGAAGACTTAAAATTTTGCTACAGG
AATAGGCCTTGGCCATGTTAAATTTTCCCCCAAATTCTAAGCTTTTTGAGATCTTGAATT
AGGGGGAAAATTCTAACATTACCATGGTGATTTAGTCAATGGCTACTGAGAACTGTAGT
TTGTGCATAATTAAGTAGTTGATGCTTTTGAGCTGCTTCTTATAATGT
>hsa-mir-106b_21_B2M-exon4
[46]
TGCTGGCTATCCTGCGCCTTTCCACTGCTCTGGTAAGTGCCCAAATTGCTGGAGGGCC
ATCTGTTTTGACCCTTAAAGGGGTAGCTCCTTACCGTGCTCTCATTGCCGCCTCCCCAC
CTCCCGCTCCAGCCCTGCCGGGGCTATATGACAAAATGTTTCATTCGTGGTCCTCTCC
GTGCGACCAAACATTGTGTCATAGCTGCTCCAGCAGGGCACGCACAGCGTCCGTGGA
GGGAAAGGCCTTTTCCCCACTTCTTAACCTTCACTGAGAGGGTGGTTGGGGTCTGTTTC
ACTCCATGTGTCCTAGATCCTGTGCTACAGACCTTCCTTTCTGTCCTCCCG
>hsa-mir-191_8_B2M-exon2-2
[47]
GTCTTGTTCCCTCTAGACTCCGTTTCACAACCTACTCCCGGGTCTTCCTCCTGGGACTG
GGGATGGGCGGGTTCTGATGTGGCCCCAGGGCGAGTGACCTGGGGGCAGGAGCTCC
CCCGCCCCCCGCCAACGGCTGGACAGCGGGTTCAGTGTAGTACAAGAGATAGATTGTC
TCCAGAGCATTCTCTATCTGCTGACTACACTGCCCCTGCTCTCCTGCCTGAGCAGCGC
CCTGGCCCAGATGGGGTGCCCCTGACCCCCAGACATACTTTACTGAGCTGCTTGGGTC
TCAGTTCCTCTCAGTTGCGCCCTCAGGCTGGAGGTGATGGGTGTAGACGTGGGAGAG
CCGAGG
>hsa-mir-191_26_B2M-exon4
[48]
GTCTTGTTCCCTCTAGACTCCGTTTCACAACCTACTCCCGGGTCTTCCTCCTGGGACTG
GGGATGGGCGGGTTCTGATGTGGCCCCAGGGCGAGTGACCTGGGGGCAGGAGCTCC
CCCGCCCCCCGCCAACGGCTGGACAGCGGGTACTTTATCAAATGTATAAGAAGTTGTC
TCCAGAGCATTCCTTCTTAGCCATTGATAAAGCCCCTGCTCTCCTGCCTGAGCAGCGCC
CTGGCCCAGATGGGGTGCCCCTGACCCCCAGACATACTTTACTGAGCTGCTTGGGTCT
CAGTTCCTCTCAGTTGCGCCCTCAGGCTGGAGGTGATGGGTGTAGACGTGGGAGAGC
CGAGG
>hsa-mir-744_47_B2M-exon4
[49]
CATTAACATGTTTTAAACTTCAGGCCCTTCTACTGCCAAGGTGAGTTCAGGCTGGGCGG
CTGCACCCCTGGGAGCAGGGCAGTGCTGCACTGAGCCAGGCGGGAGCTGGAAGAAG
ACGCAGCACACTGGGTTGGGCAAGGTAACTATCTTAACAAGCTTTGAGTCTTACTGAAG
GTTTCCTGGAAACCACGCACATCAAAGCTGTATAAAATAGAACCTTACTCGGTCCTGAC
CGGCTCGGCTTCTGTTTGTTTATTTCATCTCTACTCAGTACTGCCCTGTTCCCTGGTTTT
AAAGTTGTACTGAAATGCATACCTTGTGATAATGTTGTCACATTTTGTCCTTATTGTGTC
GTGCC
>hsa-mir-518b_43_B2M-exon4
[50]
TTTATGTTCTGGATTCCAGAAAACATGCAAACAGGGCAAATAAATGCATCTTTATTTTGT
GTCCATTTTAACCTGGTCAAGGAAAATTCCAACAGCAACATCAAAAAACCAGTGTTGGA
GCAAGAATATGTCATGCTGTGGCAGTTCAGCGTGAATAAGTTTCAATTGTCTGAAAGAA
AATTAACTTATGCACGCTTAACTATTACGGTTTGAGTAAAGCAGCGTTGAAGTTGATGCT
GATCTTGGTAATACATTTGCAGAGCGTGCTTATCATCAGACGTGGACGATGGTGGGGTT
CTGTTTTGGTTTTGTTTTTTTCTAAGACAGGGTCTCCGTTGCCCAG
>hsa-mir-99b_50_B2M-exon4
[51]
CTTCTGGGTTCTTTAGGGAGGAGGGGGATGAGAGCCTTGACTCCAGGGTCCCTGATGA
GGAAGGGGCTGAGGGCCTGGACTCCTGGGTTCCTTGGGGAGGAGGGGCCGGGGGCC
CGGACTCCTGGGTCCTGGCACCTTTATCAAATGTATAAGAAGTAGGGCCTTCGCTACTC
ACATCCTATTGATTTGATAACCGTGTCGGGGGCTCACCATCGCGGCTGGGGCCTCCCC
GGCCCTCCCCCTCATCCCTGGTCCTCCTGGTCCCTGTCTGTCTGTCTGTCGGGTCTGT
CCACC
>hsa-mir-302a_23_None (negative control)
[56]
GCCAGCACACCTTTTGTTACAAAATTTTTTTGTTATTGTGTTTTAAGGTTACTAAGCTTGT
TACAGGTTAAAGGATTCTAACTTTTTCCAAGACTGGGCTCCCCAACCCTTTAACTAAACA
ACAACAAACAGGAAACAAAATTGTTTGTTGTGGTTTAGTTGTGGGGATGGTAAGTCTTC
CTTTTACATTTTTATTATTTTTTTAGAAAATAACTTTATTGTATTGACCGCAGCTCATATAT
TTAAGCTTTATTTTGTATTTTTACATCTGTTA
>hsa-mir-200c_13_None (negative control)
[57]
AGCAGGGCTCACCAGGAAGTGTCCCCAGGGACTCGGGTGGTGGGGGGATGGGAGCC
AGGGATCTGCAGCTTTTCCGCAGGGATCCTGGGCCTGAAGCTGCCTGACCCAAGGTG
GGCGGGCTGGGCGGGGGCCCGTTCCGATTTAACGACGTAATGGGTGCGGTTGGGAGT
CTCTTTACGTCGATAAATCAGGACACGGCCCCTGTCCCTGTGTCAGCAACATCCATCGC
CTCAGGTCCCCAGCCCTTAGCTGGCTGCAGCCCCCTCCCCACTTCCCACGCACCCCG
GAAGCCCCTCGTCTTGAGCTGAGAGCGTTGCACAAGGGGTGG

In addition to the negative control constructs listed in Table 1 above, an additional miR30-based construct was purchased from Vector Builder (Neg Ctrl 0).

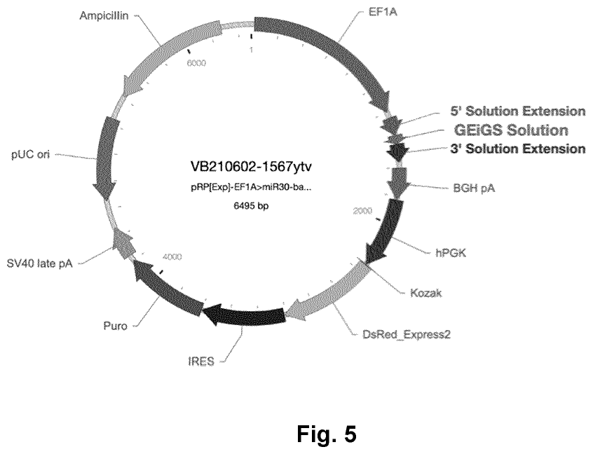

The extended solutions were synthesised and cloned into a bespoke expression plasmid (plasmid VB210602-1567ytv, see FIG. 5). The plasmid is a mammalian expression system in which the EF1 alpha (EF1a) promoter drives transcription of the extended GEiGS solution. On the same plasmid DsRed Express 2 (DsRed) cDNA is expressed from the human PGK (hPGK) promoter. Upon transfection into mammalian/human cells, both the GEiGS solution and DsRed cDNA were transcribed. DsRed can be used as a transfection marker, indicating the successfully transfected cells. The level of DsRed expression in the cell (fluorescence intensity) can be assayed by flow cytometry and can be used as a proxy for the expression level/abundance of the GEiGS solution.

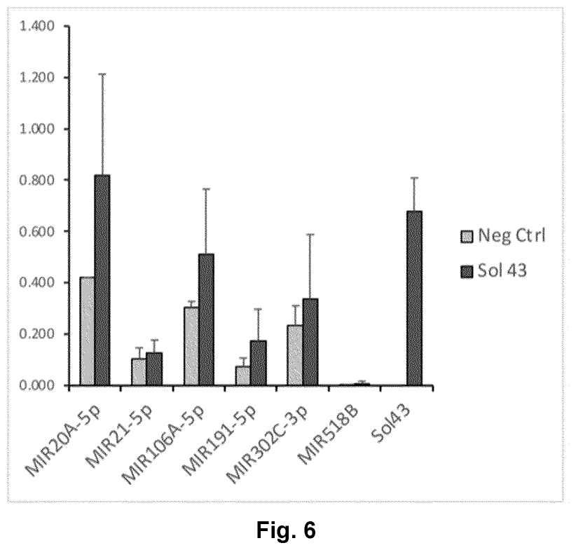

The cloned solutions were individually transfected into the human iPSCs line WTC-11 (Allen Institute of Cell Science) by nucleofection (Lonza). These are undifferentiated iPSCs. The expression of the GEiGS sRNA within the transfected cells from the vector was measured by qPCR and compared to the expression of the corresponding endogenous miRNA scaffold. This showed that the GEiGS sRNA was expressed at comparable levels to the endogenous miRNA scaffold (FIG. 6), indicating that expression from the plasmid is not supra-physiological, therefore unlikely to cause significant off-target effects or interfere with the normal functions of the RNAi pathway in the cell.

To assess the effectiveness of solutions in silencing B2M, transfected cells were collected 72 hours post-transfection, stained for surface B2M expression with an APC-coupled antibody, and analysed by flow cytometry. If the GEiGS solution design is effective, transcription of the extended GEiGS solution should lead to its successful sequential post-transcriptional processing by DROSHA and DICER, expression of the mature GEiGS silencing RNA, incorporation into RISC and ultimately B2M mRNA degradation. Validation of the efficiency of post-transcriptional processing of the extended GEiGS solution, the efficiency of incorporation into RISC, and the potential of the GEiGS sRNA to trigger B2M mRNA degradation was assessed.

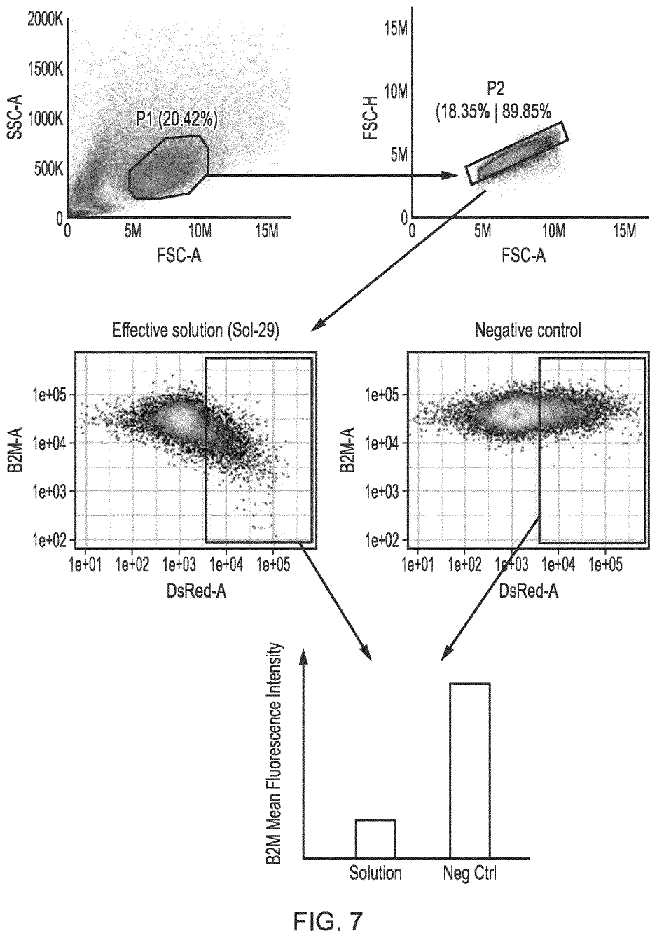

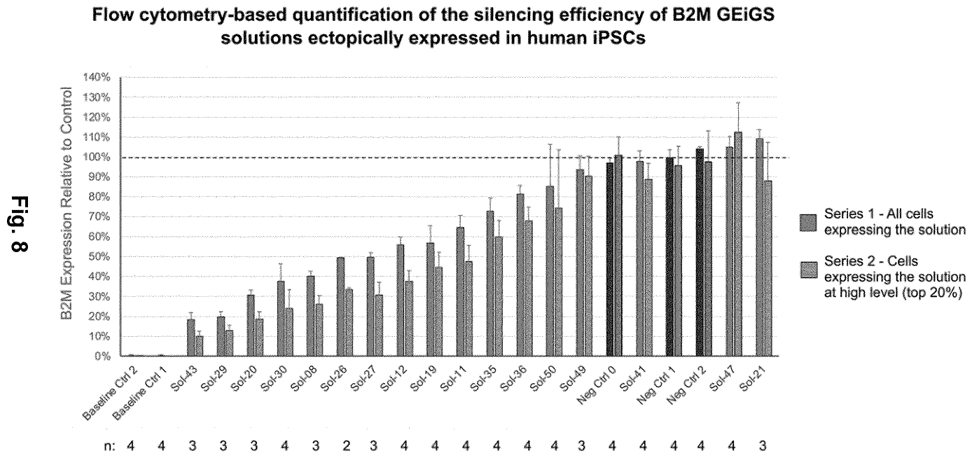

By using flow cytometry to concomitantly measure the expression of DsRed (transfection and solution expression reporter) and B2M (GEiGS solution target) we were able to generate dose-dependent curves showing B2M residual expression vs solution abundance in the cell (FIG. 7). As expected, for negative control constructs the level of B2M did not change irrespective of the level of expression of the GEiGS solution. For effective solutions, B2M levels decreased in direct correlation with the levels of the solution expression in the cell. In other words, cells in which transfection led to higher levels of DsRed/solution expression showed reduced level of B2M.

The efficiency of the GEiGS solutions was quantified through the flow cytometry gating shown in FIG. 7. Debris were gated out using the FSC-A and SSC-A flow cytometry channels, and subsequent doublets were gated out using the FSC-A and FSC-H channels. Filtered data was subsequently plotted as B2M vs DsRed. The quantification was performed in two different ways:

Placing a gate on all the cells expressing DsRed (when compared to an untransfected control), and then extracting a mean value for the B2M fluorescence intensity in this subpopulation.

Placing a more selective gate solely on the cells expressing DsRed at high level (top 20%), and subsequently calculating the mean fluorescence intensity for B2M in this subpopulation.

Both approaches were in agreement and allowed comparison of the effectiveness of the different B2M-targeting GEiGS solutions. Between the 17 solutions tested experimentally, a broad spectrum of B2M silencing was achieved, ranging from 10%-90% (FIG. 8). The most effective solutions showed strong silencing activity. When expressed at moderate levels, 2 of the 17 solutions tested knocked down endogenous B2M by >80%, and 4 of the 17 knocked down endogenous B2M by >65%. When expressed at high levels, 3 of the 17 solutions tested knocked down endogenous B2M by >80%, and 7 of the 17 solutions tested knocked down endogenous B2M by >65%. Solution activities were reproducible between experimental replicates (mean CV=14%, median CV=11%) indicating this assay was robust.

This set of solutions can therefore be used to achieve distinct levels of MHC-I silencing over a broad range of MHC-I expression in engineered cells, allowing the identification of MHC-I levels that can evade both adaptive and innate immune responses post-transplantation in the host.

### Example 3—Gene Editing of the Endogenous miRNA Scaffold in the Desired Cell Type

Solutions 8, 20, 29 and 30 (see Table 1) were implemented as full GEiGS, i.e. by editing the endogenous loci of the respective miRNA scaffolds. Gene editing was performed by CRISPR knock-in using the following gRNA and HDR template sequences:

TABLE 2

| | gRNA | | |
|---|---|---|---|
| Solution | sequence | | Type |
| (miRNA | [SEQ ID | | of HDR |
| scaffold) | NO: #] | HDR template sequence [SEQ ID NO: #] | donor |
| Sol-8 | GGATTCCG | CCTCAGTCAAACTGAGGACTTCAGATACTCTCATC | ssDNA |
| (hsa-mir- | TTGCCCGC | CACGAAGACGGTTGGGGTGGTTTTTCCCCAGGAA | |
| 191A | TGTCC | GTAAGAGGGCTATCTTTAGCGAGATGCGGGCTGA | |
| | [192] | GGCAGCCCTGGGGGAGTCACTACCATTGCAGCCC | |
| | | TACCCAGAGGTCCTGGGTCCACCCTCCCTCGCCA | |
| | | ATCCTCCCCAGGGTTCCAGAGATGGCCACCAGCC | |
| | | TCGGCTCTCCCACGTCTACACCCATCACCTCCAGC | |
| | | CTGAGGGCGCAACTGAGAGGAACTGAGACCCAAG | |
| | | CAGCTCAGTAAAGTATGTCTGGGGGTCAGGGGCA | |
| | | CCCCATCTGGGCCAGGGCGCTGCTCAGGCAGGA | |
| | | GAGCAGGGGCAGTGTAGTCAGCAGATAGAGAATG | |
| | | CTCTGGAGACAATCTATCTCTTGTACTACACTGAA | |
| | | CCCGCTGTCCAGCCGTTGGCGGGGGGCGGGGGA | |
| | | GCTCCTGCCCCCAGGTCACTCGCCCTGGGGCCAC | |
| | | ATCAGAACCCGCCCATCCCCAGTCCCAGGAGGAA | |
| | | GACCCGGGAGTAGGTTGTGAAACGGAGTCTAGAG | |
| | | GGAACAAGACGCTCACCTGGGCCACAGAACAATA | |
| | | GAAGGGGAGGTGGGGGCCTCCCCTGGGACCGGG | |
| | | TCCCGATCCGGATTAGATCCGTGGGGCAGGATTA | |
| | | GCCCTGTTGGTCCCACGAAGCCTGGGCCACTGGT | |
| | | GCGGTGACGGTGGTGGGGCGGGCTTCGGAATCC | |
| | | GTCAGGAGGGAAGCGCGCCCGTGGGGCCGGAGA | |
| | | GGAAGTAAACCCGC [193] | |
| Sol-20 | GCACTGTAA | TATGAGCACTTTGGTACTACTAGGACCCAATCTTATCCT | ssDNA |
| (hsa-mir- | GCACTTTTA | CTGTACACAACTGTACACTGCACAGTCCCCACCATCTGT | |
| 106a) | CA [58] | TCCCAACAAACAATAAAAAGGCAAAAAACAAACCACTGA | |
| | | AATGTGTCCTATTCAAATTACTACTCAAGGCATACTTTTA | |
| | | AAATGCTGTATATTAGGCAGCCTGTGCCAGAAGGGGCA | |
| | | TTTAGGGCAGTAGATTCTAAGCTGCTTCACTAACTGCAC | |
| | | TAGATGCACCTTAACACAAGAGACACATTATAAGAAGCA | |
| | | GCTCAAAAGCATCAACTACTTAATTATGCACAAACTACA | |
| | | GTTCTCAGTAGCCATTGACTAAATCACCATGGTAATGTT | |
| | | AGAATTTTCCCCCTAATTCAAGATCTCAAAAAGCTTAGAA | |
| | | TTTGGGGGAAAATTTAACATGGCCAAGGCCTATTCCTGT | |
| | | AGCAAAATTTTAAGTCTTCCAGGAGCTCTTCTTTTCCGTC | |
| | | CTCACAGATCCATGCAGACTCACCTGGTTAAAACAACAA | |
| | | AACAAACACACACATATTAGAAACTAAACAAGTAAACTAC | |
| | | TAGTTAATATTCCTGAAACAGGCTTTCCTTTGGCTTACAG | |
| | | AGTCTAAGCTAGGTTTTGTGGTTTCAACCAAATCCTGAG | |
| | | AAAT [59] | |
| Sol-29 | CACTAAAGT | AGGGCCTGCTGATGTTGAGTGCTTTTTGTTCTAAGGTGC | ssDNA |
| (hsa-mir- | GCTTATAGT | ATCTAGTGCAGATAGTGAAGTAGATTAGCATCTACTGCC | |
| 20a) | GC [60] | CTAAGTGCTCCTTCTGGCATAAGAAGTTATGTATTCATC | |
| | | CAATAATTCAAGCCAAGCAAGTATATAGGTGTTTTAATAG | |
| | | TTTTTGTTTGCAGTCCTCTGTTAGTTTTGCATAGTTGCAC | |
| | | TACAAGAAGAATGTAGTTGTGCAAATCTATGCAAAACTG | |
| | | ATGGTGGCCTGCTATTTCCTTCAAATGAATGATTTTTACT | |
| | | AATTTTGTGTACTTTTATTGTGTCGATGTAGAATCTGCCT | |
| | | GGTCTATCTGATGTGACAGCTTCTGTAGCACTAACAAAT | |
| | | TTCCAATAATCCTGTTGTTTAGTTATACAGATTATTGGAA | |
| | | ATTTGTAAAGTACTGCTAGCTGTAGAACTCCAGCTTCGG | |
| | | CCTGTCGCCCAATCAAACTGTCCTGTTACTGAACACTGT | |
| | | TCTATGGTTAGTTTTGCAGGTTTGCATCCAGCTGTGTGA | |
| | | TATTCTGCTGTGCAAATCCATGCAAAACTGACTGTGGTA | |
| | | GTGAAAAGTCTGTAGAAAAGTAAGGGAAACTCAAACCCC | |
| | | TTTCTACACAGGTTGGGATCGGTTGCAATGCTGTGTTTC | |
| | | TGTATGGTATTGCACTTGTCCCGGCCTGTTGAGTTTGGT | |
| | | GGGGATTGTGACCAGAAGATTTTGAAAATTAAATATTACT | |
| | | GAAGATTTCGACTTCCACTGTTAAATGTACAAGATACAT | |
| | | GAA [61] | |

Sol-30 gRNA and HDR template sequences is shown in Table 3.

The gRNAs were obtained from Integrated DNA Technologies (IDT) as synthetic sgRNAs. Cas9 protein was also obtained from IDT. sgRNA/Cas9 ribo-nucleoproteins (RNP) were transfected into iPSCs using nucleofection. Five days post-transfection CRISPR pools were cloned in 96-well plates using the F.SIGHT platform (Cytena), and single cell deposition was confirmed using the NYONE imaging system (Synentec). Cell colonies were expanded for 7-10 days and subsequently were genotyped by PCR, sanger sequencing and NGS amplicon sequencing. Confirmed GEiGS lines were analysed by flow cytometry to assess the level of B2M expression.

For solution 29, both heterozygous (WT/KI) and homozygous (KI/KI) clones were isolated, thus expressing the sRNA mono- and bi-allelically, respectively. Because MIR106A is X-linked (hemizygous), the GEiGS clones isolated for solution 20 were only expressing the sRNA mono-allelically. For solution 30 homozygous (KI/KI) clones were generated more efficiently with Cas12a (Cpf1) protein (also from IDT) instead of Cas9.

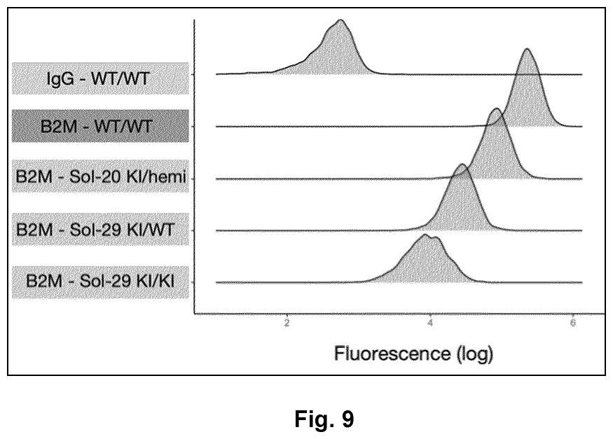

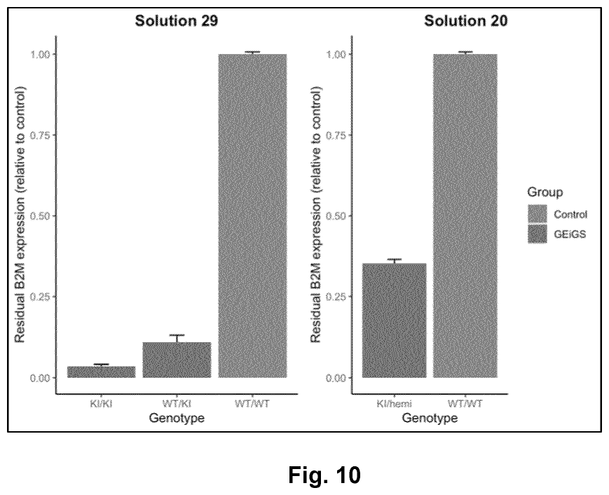

GEiGS lines showed homogenous silencing of B2M, with very similar variance of B2M expression between the control parental line (WT/WT) and the GEiGS lines (FIG. 9). Quantification of B2M silencing in multiple clones, showed consistent B2M silencing between different clones of the same genotype, reflecting the reproducible silencing activity of GEiGS (FIG. 10). On average, heterozygous WT/KI clones for solution 29 showed silencing of B2M by 89%±SD 2% (n=4), while homozygous KI/KI clones for the same solution showed silencing by 96%±SD 0.5% (n=2) (FIG. 10). For solution 20, hemizygous KI clones showed B2M silencing by 65%±SD 1.2% (n=4) (FIGS. 9 and 10) and for solution 30 a homozygous KI clone displayed approximately 31% level of B2M silencing (n=1).

### Example 4—Gene Editing of the Endogenous miRNA Scaffold in Primary Human T Lymphocytes

Solution 30 (see Table 3) is implemented as full GEiGS i.e. by editing the endogenous loci of the respective miRNA scaffolds. A number of stimulation-induced miRNAs can be detected in primary T cells (see Amarel et al. 2017—EMBO J. 2017 Feb. 1; 36(3):346-360. Doi: 10.15252/embj.201694335). Gene editing is performed by CRISPR knock-in using a methodology described in the paper by Roth et al. 2018 (Nature. 2018 July; 559(7714):405-409. Doi: 10.1038/s41586-018-0326-5). The following gRNA and HDR template sequences are being used:

TABLE 3

| Solution | | | Type |
|---|---|---|---|
| (miRNA | gRNA | | of HDR |
| scaffold) | sequence | HDR template sequence | donor |
| Sol-30 | ATGTTGACT | AGCATGTACTCTGGTTTCAACAGACACAAATTTATATGTTA | dsDNA |
| (hsa-mir- | GTTGAATCT | ACCCAGTTTTCTTGCCGTTCTGTAAGTGTTTTATTCTTAGT | |
| 21) | CA [62] | GTGATTTTTTTCCATTGGGATGTTTTTGATTGAACTTGTTC | |
| | | ATTTTGTTTTGCTTGGGAGGAAAATAAACAATTTTACTTTTT | |
| | | TCCTTTAGGAGCATTATGAGCATTATGTCAGAATAGAATAG | |
| | | AATTGGGGTTCGATCTTAACAGGCCAGAAATGCCTGGGTT | |
| | | TTTTTGGTTTGTTTTTGTTTTTGTTTTTTTATCAAATCCTGC | |
| | | CTGACTGTCTGCTTGTTTTGCCTACCATCGTGACATCTCC | |
| | | ATGGCTGTACCACCTTGTCGGGTGTTAACATTATTATAAC | |
| | | CCTACTGTTGAATCTCATGGAGGGTCATAATATGTTAACAT | |
| | | CTGACATTTTGGTATCTTTCATCTGACCATCCATATCCAAT | |
| | | GTTCTCATTTAAACATTACCCAGCATCATTGTTTATAATCA | |
| | | GAAACTCTGGTCCTTCTGTCTGGTGGCACTTAGAGTCTTT | |
| | | TGTGCCATAATGCAGCAGTATGGAGGGAGGATTTTATGGA | |
| | | GAAATGGGGATAGTCTTCATGACCACAAATAAATAAAGGA | |
| | | AAACTAAGCTGCATTGTGGGTTTTGAAAAGGTTATTATACT | |
| | | TCTTAACAATTCTTTTTTTCAGGGACTTTTCTAGCTGTATG | |
| | | ACTGTTACTTGACCTTCTTTGAAAAGCATTCCCAAAATGCT | |
| | | CTATTTTAGATAGATTAACATTAACCAACAT [63] | |

The gRNAs are obtained from Integrated DNA Technologies (IDT) as synthetic sgRNAs. Cas9 protein is also obtained from IDT. sgRNA/Cas9 ribo-nucleoproteins (RNP) are transfected into primary human T cells using electroporation. The method used in Roth et al. (2018) preferably recommends fresh cells, bulk T cells or, or sub-populations sorted by fluorescence activated cell sorting (FACS), and with cells from whole blood or leukapheresis, though it also is used with cells that have been recovered from cryo-preservation.

Immediately after isolation, T cells are stimulated for 2 days with anti-human CD3/CD28 magnetic dynabeads (ThermoFisher) at a beads to cells concentration of 1:1, along with a cytokine cocktail of IL-2 at 200 U ml⁻¹, IL-7 at 5 ng ml⁻¹, and IL-15 at 5 ng ml⁻¹, prior to electroporation in accordance with standard methods (see, e.g., Roth et al.).

After electroporation, T cells are cultured in media with IL-2 at 500 U ml⁻¹. Throughout the culture period T cells are maintained at an approximate density of 1 million cells per ml of media. Every 2-3 days after electroporation, additional media is added, along with additional fresh IL-2 to bring the final concentration to 500 U ml⁻¹, and cells are transferred to larger culture vessels as necessary to maintain a density of 1 million cells per ml.

Five days post-transfection GEiGS-modified cells are analysed by flow cytometry to assess the level of B2M expression. Cells are also analysed by PCR, sanger sequencing and NGS amplicon sequencing to assess the efficiency of gene editing. At this stage, B2M-low cells can be FACS-sorted to enrich the fraction of GEiGS-modified cells.

### Example 4.1—Gene Editing of the Endogenous miRNA Scaffold in Primary Human T Lymphocytes

Solution 30 (see Example 2 and Table 3) was implemented as full GEiGS i.e. by editing the endogenous loci of the respective miRNA scaffolds. A number of stimulation-induced miRNAs can be detected in primary T cells (see Amarel et al. 2017—EMBO J. 2017 Feb. 1; 36(3):346-360. doi: 10.15252/embj.201694335).

Gene editing was performed by CRISPR knock-in using a methodology described in the paper by Roth et al. 2018 (Nature. 2018 July; 559(7714):405-409. doi: 10.1038/s41586-018-0326-5).

The method used in Roth et al. (2018) preferably recommends fresh cells, bulk T cells or, or sub-populations sorted by fluorescence activated cell sorting (FACS), and with cells from whole blood or leukapheresis, though it also is used with cells that have been recovered from cryo-preservation.

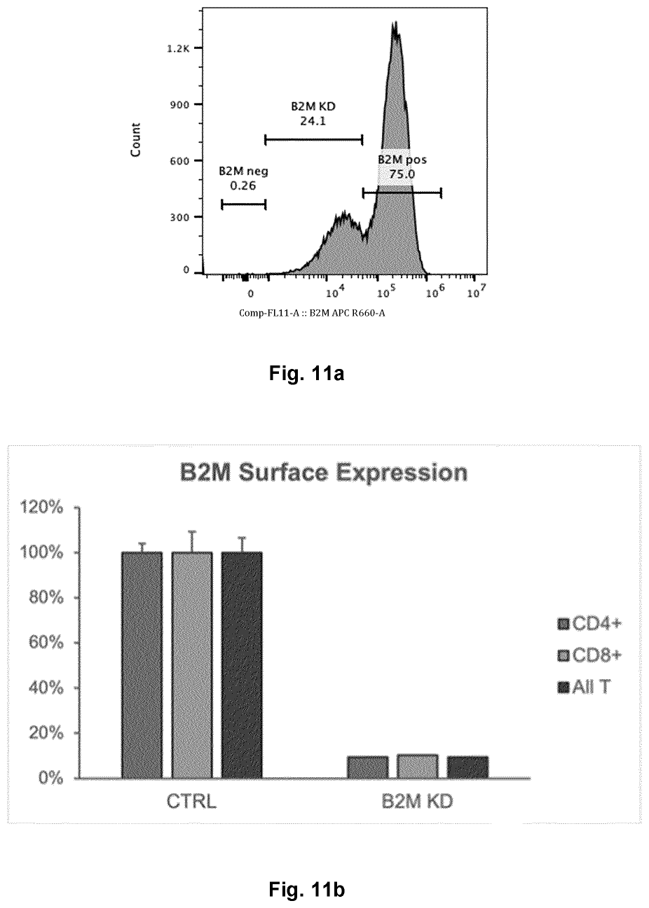

The gRNAs for Solution 30 were obtained from Integrated DNA Technologies (IDT) as synthetic sgRNAs. Cas9 protein was obtained from IDT. Immediately after isolation from donor peripheral blood mononuclear cell isolates (PBMC), T cells were stimulated for 2 days with anti-human CD3/CD28 magnetic dynabeads (ThermoFisher) at a beads to cells concentration of 1:1, along with a cytokine cocktail of IL-2 at 200 U ml⁻¹, IL-7 at 5 ng ml⁻¹, and IL-15 at 5 ng ml⁻¹. Beads were removed prior to electroporation in accordance with standard methods (see, e.g., Roth et al.). The sgRNA/Cas9 ribo-nucleoproteins (RNP) were transfected into primary human T cells using electroporation. After electroporation, T cells were cultured in media with IL-2 at 500 U ml⁻¹. Throughout the culture period T cells were maintained at an approximate density of 1 million cells per ml of media. Three days after electroporation cells were isolated for analysis of surface B2M expression using flow cytometry and gDNA and RNA extracted for PCR, Sanger sequencing and NGS amplicon sequencing to assess the efficiency of gene editing. Results indicate a reduction in surface B2M expression (FIG. 4). Of the CD3 positive, mixed T cell population approximately 24% of the cells demonstrated a silencing of B2M (FIG. 11a) with a reduction in mean fluorescent intensity of 80% (n=1), compared with control unedited cells. Analysis confirms that both CD4 and CD8 edited T cells demonstrated similar reduction in B2M surface expression (FIG. 11b).

### Example 5—Demonstrating Evasion of Both T Cell and NK-Cell Mediated Immune Responses in Culture to Clonal GEiGS-Modified Cell Lines

To determine whether partial silencing of B2M enables cells to evade both adaptive and innate immune cell responses, published methods are in use to establish the reactivity in cell culture of donor-derived cytotoxic T-lymphocytes (CTLs) or NK (Natural Killer or LAK, lymphokine activated killer) cells to the GEiGS-modified cell lines (Haga K et al. 2006, Hacke K et al. 2009, Han X et al. 2019). Donor CTLs and NK cells are conveniently isolated from commercial suppliers of peripheral blood mononuclear cells (PBMC).

T lymphocytes are initially preactivated in tissue culture to make them allo-reactive. This is performed via stimulation with cells, cell extracts or stimulated cells presenting HLA-antigens which are recognised as foreign (as they are un-matched) by the donor T lymphocytes. Once activated the donor cells are then mixed in culture with a GEiGS-modified cell line at a defined ratio (typically 10 donor cells:1 GEiGS-modified cell), though other ratios are tested. After a defined period in mixed cell culture (48 hrs), effector donor T lymphocyte activation by the GEiGS-modified cell line is analysed by measuring proliferation, or inflammatory cytokine secretion (measured by ELISA), or the extent of GEiGS-modified cell cytotoxicity mediated by the T lymphocytes is assessed by quantifying GEiGS-modified cell line death by measuring release of the cytoplasmic enzyme lactate dehydrogenase into the culture medium, and/or release of pre-loaded Calcein AM, and/or staining with a cell death marker (e.g. propidium iodide), and/or time-course visualisation of GEiGS-modified cells preloaded with a fluorescent marker (e.g., CFSC). Undifferentiated iPSC lines (knock-in for Solution 20 or 29), iPSC lines differentiated into pancreatic progenitor cells (knock-in for Solution 30) do not express quantitatively the same level of B2M as the parental iPSC line.

NK cells are non-HLA-restricted and potent effectors which recognise and kill cells that are deficient in surface expression of MHC-I (‘missing self’), such as malignant cells. In a related co-culture assay using NK cells from the same PBMC donor, these are mixed in culture with a GEiGS-modified cell line at a defined ratio (typically 5:1 or 1:1). After a defined period in mixed cell culture (4-48 hrs), the extent of GEiGS-modified cell cytotoxicity mediated by the NK cells is assessed by quantifying GEiGS-modified cell line death (as above). Undifferentiated iPSC lines (knock-in for Solution 20 or 29), iPSC lines differentiated into pancreatic progenitor cells (knock-in for Solution 30) do not express quantitatively the same level of B2M as the parental iPSC line.

Hence, we demonstrate that efficient and dose-dependent reduction in surface expression of HLA in human cells, yields enhanced resistance to allo-reactive T lymphocyte mediated cytotoxicity, while avoiding non-MHC restricted killing.

### Example 6—Demonstrating Evasion of NK-Cell Mediated Immune Responses in Culture to Clonal GEiGS-Modified Cell Lines

Natural Killer (NK) cells recognise and kill cells which are deficient in surface expression of MHC-I (‘missing self’). NK cell assays were set up to determine how their killing activity against GEiGS-modified iPSC was modulated by B2M silencing.

Pre-isolated CD56+NK cells were thawed and stimulated to proliferate in IL-2/IL-12, IL-2 alone or without cytokines. On the day of the assay, NK cells were counted and stained with CellTrace™ CFSE Cell Proliferation Kit. Target cells included K562 cells as a positive control for cell killing. It is known that the tumour cell line K562 serves as a control to assess the extent of activated NK cell mediated killing as it is known to be a potent stimulator of activated NK cell mediated killing (Zamai L, A R Mariani, G Zauli, L. Rodella, R. Rezzani, F A Manzoli and M Vitale. Kinetics of in vitro natural killer activity against K562 cells as detected by flow cytometry. 1998. Cytometry 32(4):280-285. DOI: 10.1002/(sici)1097-0320(19980801)32:4<280::aid-cyto4>3.0.co;2-m).

NK cells were activated with the cytokines IL-2 or IL-12, or a mixture of IL-2 and IL-12 prior to mixing the cells at a defined ratio (typically 5:1 or 1:1). Control (unmodified) iPSC, B2M-iPSC and GEiGS-modified iPSC, were stained with Zombie NIR. The Zombie stain allows for differentiating between cells that died before the start of the killing assay as opposed to cells that died during the course of the assay. Target cells were typically seeded into a 96 well plate at a density of 40 000 cells/well.

NK cells were then added to the wells at varying ratios to the target cells. Typical ratios evaluated were 5(NK):1(target) or 2.5:1 or 1:1. Extra wells of target cells were included from which the maximal and spontaneous release could be calculated at the end of the assay. The plate was then incubated for a defined period (4-48 hours) at 37° C., 5% CO₂.

Following incubation, maximal release was determined by lysing samples with 20% Triton-X. All wells were then stained with Propidium iodide (PI). Once the staining was complete the plate was run on the flow cytometer. CFSE staining allowed us to identify the NK cells, Zombie NIR stained cells that died prior to the killing assay, while PI stained cells that had died during the killing assay. Spontaneous release values were subtracted from the PI values and the remaining amount was the percentage of target cells killed by the NK cells.

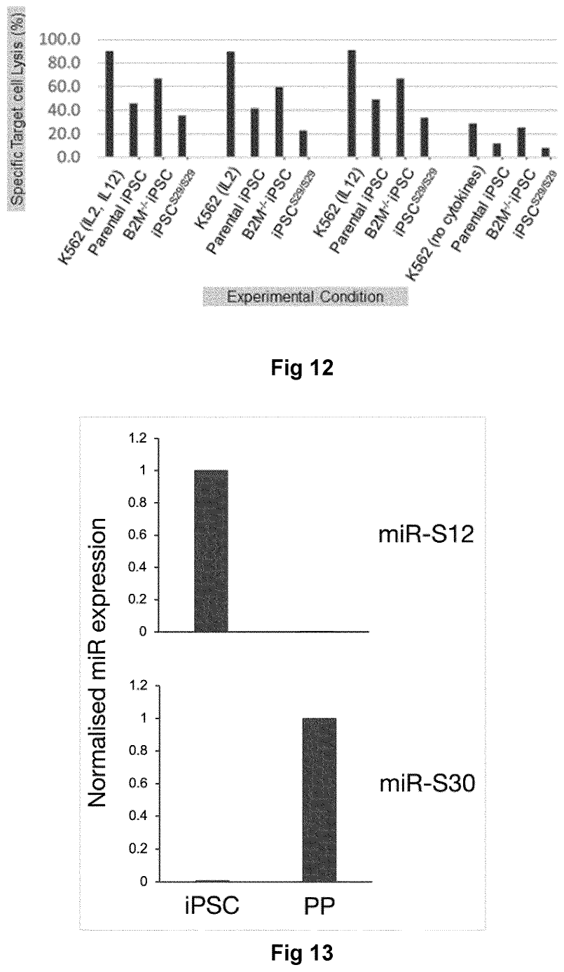

Specific lysis was observed against K562 cell line in the presence or absence of cytokine stimulation, as a positive control (FIG. 12). Control iPSC displayed a reduced level of specific lysis (approximately 50% of the maximum level of cell killing observed for K562 cells), whereas as expected, undifferentiated B2M^{-/-} iPSC showed elevated levels of specific lysis (FIG. 12). This is because complete absence of B2M results in complete absence of MHC-I (“missing self” phenotype) and these cells are effectively detected and lysed by NK cells. Undifferentiated, GEiGS-modified iPSC lines (iPSCs29i29), did not express quantitatively the same level of B2M as the control iPSC line from which it is derived (i.e. they express lower levels of B2M and thus lower levels of MHC-I cell surface expression) and therefore displayed consistently reduced specific lysis by NK cells compared the B2M^{-/-} cell lines across all mixed culture conditions tested.

This demonstrates that partial knockdown of B2M results in partial reduction of MHC-I cell surface expression which in turn allows the cells to evade the innate (NK-mediated) immune system shown by reduced specific lysis of the GEiGS-modified iPSC lines (iPSC^{S29/29}) cells by activated NK-cells in tissue culture. Therefore, partial knockdown of B2M results in enhancing cell survival following exposure to cells of the innate (NK-mediated) immune system.

### Example 7—Selection of GEiGS Solutions for Partial Silencing of B2M Using miRNA Scaffolds that are Expressed in a Context-Specific Manner for Validation Via Plasmid-Based Ectopic Expression and GEiGS

TABLE 4
Sequences of computationally-designed B2M-targeting RNAi trigger sequences and
GEiGS solutions based on miRNAs expressed more highly in differentiated cells
(pancreatic and primary human T cells):

| GEiGS | | GEiGS mature sRNA | GEiGS solution sequence |
|---|---|---|---|
| Solution | miRNA | sequence (guide) | (pre-miRNA hairpin) |
| ID | scaffold | [SEQ ID NO: #] | [SEQ ID NO: #] |
| Sol-8 | hsa-mir- | TTCAGTGTAGTACAAG | CGGCTGGACAGCGGGttcagtgtagtacaagag |
| | 191 | AGATAGA [1] | atagaTTGTCTCCAGAGCATTCTCTATCTG |
| | | | CTGACTACACTGCCCCTGCTCTCCTGCC |
| | | | T [18] |
| Sol-30 | hsa-mir- | TGTTAACATTATTATA | TGTCGGGtgttaacattattataaccctacTGTTGAA |
| | 21 | ACCCTAC [10] | TCTCATGGAGGGTCATAATATGTTAACAT |
| | | | CTGACA [27] |
| Neg Ctrl | hsa-mir- | TTAACTAAACAACAAC | CCAACCCTttaactaaacaacaacaaacagGAAA |
| 1 | 302a | AAACAG [52] | CAAAATTGTTTGTTGTGGTTTAGTTGTGG |
| | | | GGATGG [53] |
| Neg Ctrl | hsa-mir- | TTTACGTCGATAAATC | CCCGTTCCGATTTAACGACGTAATGGGT |
| 2 | 200c | AGGACAC [54] | GCGGTTGGGAGTCTCtttacgtcgataaatcagg |
| | | | acacGG [55] |

A number of the extended designs tested were based on endogenous pre-miRNAs expressed more highly in undifferentiated iPSC rather than differentiated progeny from the stem cells. These solutions are listed as follows:

TABLE 5
Sequences of computationally-designed B2M-targeting RNAi trigger sequences and
GEiGS solutions based on miRNAs expressed highly in undifferentiated iPSC:

| GEiGS | | GEiGS mature sRNA | GEiGS solution sequence |
|---|---|---|---|
| Solution | miRNA | sequence (guide) | (pre-miRNA hairpin) |
| ID | scaffold | [SEQ ID NO: #] | [SEQ ID NO: #] |
| Sol-12 | hsa-mir- | TTCAGTAAGTCAAC | CCTACATTTTAGTTGACTTACTGCAG |
| | 302c | TTCAATGT [3] | CTGTGTGAAACAAAAGTttcagtaagtcaa |
| | | | cttcaatgtAGG [20] |
| Sol-20 | hsa-mir- | TAAATTTTCCCCCA | CCTTGGCCATGTtaaattttcccccaaattctaa |
| | 106a | AATTCTAAG [5] | gCTTTTTGAGATCTTGAATTAGGGGG |
| | | | AAAATTCTAACATTACCATGG [22] |
| Sol-29 | hsa-mir- | TAACAAATTTCCAAT | GTAGCACtaacaaatttccaataatcctgtTGTT |
| | 20a | AATCCTGT [9] | TAGTTATACAGATTATTGGAAATTTGT |
| | | | AAAGTACTGC [26] |
| Sol-43 | hsa-mir- | TTAACTTATGCACG | TCATGCTGTGGCAGTTCAGCGTGAAT |
| | 518b | CTTAACTA [14] | AAGTTTCAATTGTCTGAAAGAAAAttaa |
| | | | cttatgcacgcttaactaTTACGGTTTGA [31] |

>hsa-mir-191_8_B2M-exon2-2
[47]
GTCTTGTTCCCTCTAGACTCCGTTTCACAACCTACTCCCGGGTCTTCCTCCTGGGACTG
GGGATGGGCGGGTTCTGATGTGGCCCCAGGGCGAGTGACCTGGGGGCAGGAGCTCC
CCCGCCCCCCGCCAACGGCTGGACAGCGGGTTCAGTGTAGTACAAGAGATAGATTGTC
TCCAGAGCATTCTCTATCTGCTGACTACACTGCCCCTGCTCTCCTGCCTGAGCAGCGC
CCTGGCCCAGATGGGGTGCCCCTGACCCCCAGACATACTTTACTGAGCTGCTTGGGTC
TCAGTTCCTCTCAGTTGCGCCCTCAGGCTGGAGGTGATGGGTGTAGACGTGGGAGAG
CCGAGG
>hsa-mir-302c_12_B2M-exon2-2
[37]
TTTACTTCTCCAAAATAGAACACGCTAACCTCATTTGAAGGGATCCCCTACATTTTAGTT
GACTTACTGCAGCTGTGTGAAACAAAAGTTTCAGTAAGTCAACTTCAATGTAGGTGTCT
CCAAGCCAGCACACCTTTTGTTACAAAATTTTTTTGTTATTGTGTTTTAAGGTTACTAAGC
TTGTTACAGGTTAAAGGATTCTAACTTTTTCCAAGA
>hsa-mir-106a_20_B2M-exon4
[45]
TTTACTTGTTTAGTTTCTAATATGTGTGTGTTTGTTTTGTTGTTTTAACCAGGTGAGTCTG
CATGGATCTGTGAGGACGGAAAAGAAGAGCTCCTGGAAGACTTAAAATTTTGCTACAGG
AATAGGCCTTGGCCATGTTAAATTTTCCCCCAAATTCTAAGCTTTTTGAGATCTTGAATT
AGGGGGAAAATTCTAACATTACCATGGTGATTTAGTCAATGGCTACTGAGAACTGTAGT
TTGTGCATAATTAAGTAGTTGATGCTTTTGAGCTGCTTCTTATAATGT
>hsa-mir-20a_29_B2M-exon4
[39]
GAATGATTTTTACTAATTTTGTGTACTTTTATTGTGTCGATGTAGAATCTGCCTGGTCTAT
CTGATGTGACAGCTTCTGTAGCACTAACAAATTTCCAATAATCCTGTTGTTTAGTTATAC
AGATTATTGGAAATTTGTAAAGTACTGCTAGCTGTAGAACTCCAGCTTCGGCCTGTCGC
CCAATCAAACTGTCCTGTTA
>hsa-mir-21_30_B2M-exon4
[41]
GTTCGATCTTAACAGGCCAGAAATGCCTGGGTTTTTTTGGTTTGTTTTTGTTTTTGTTTTT
TTATCAAATCCTGCCTGACTGTCTGCTTGTTTTGCCTACCATCGTGACATCTCCATGGCT
GTACCACCTTGTCGGGTGTTAACATTATTATAACCCTACTGTTGAATCTCATGGAGGGT
CATAATATGTTAACATCTGACATTTTGGTATCTTTCATCTGACCATCCATATCCAATGTTC
TCATTTAAACATTACCCAGCATCATTGTTTATAATCAGAAACTCTGGTCCTTCTGTCTGG
TGGCACTTAGAGTCTTTTGTGCCATAATGCA
>hsa-mir-518b_43_B2M-exon4
[50]
TTTATGTTCTGGATTCCAGAAAACATGCAAACAGGGCAAATAAATGCATCTTTATTTTGT
GTCCATTTTAACCTGGTCAAGGAAAATTCCAACAGCAACATCAAAAAACCAGTGTTGGA
GCAAGAATATGTCATGCTGTGGCAGTTCAGCGTGAATAAGTTTCAATTGTCTGAAAGAA
AATTAACTTATGCACGCTTAACTATTACGGTTTGAGTAAAGCAGCGTTGAAGTTGATGCT
GATCTTGGTAATACATTTGCAGAGCGTGCTTATCATCAGACGTGGACGATGGTGGGGTT
CTGTTTTGGTTTTGTTTTTTTCTAAGACAGGGTCTCCGTTGCCCAG

### Example 8—Demonstrating B2M Silencing in Pancreatic Progenitors Specified in Culture from Clonal GEiGS-Modified iPSC

Solution 30 (see Table 3 and Table 4) is implement as full GEiGS to demonstrate that B2M silencing can be programmed to be deployed only when therapeutic cells reach a specific stage of differentiation, as in the case of iPSC-derived pancreatic cells. The endogenous loci of the respective miRNA (hsa-mir-21) is modified (as described in Example 3), as this miRNA scaffold is known to be expressed in a developmentally regulated and cell state specific manner in the pancreatic cells (Jin W et al 2019, see also FIG. 13).

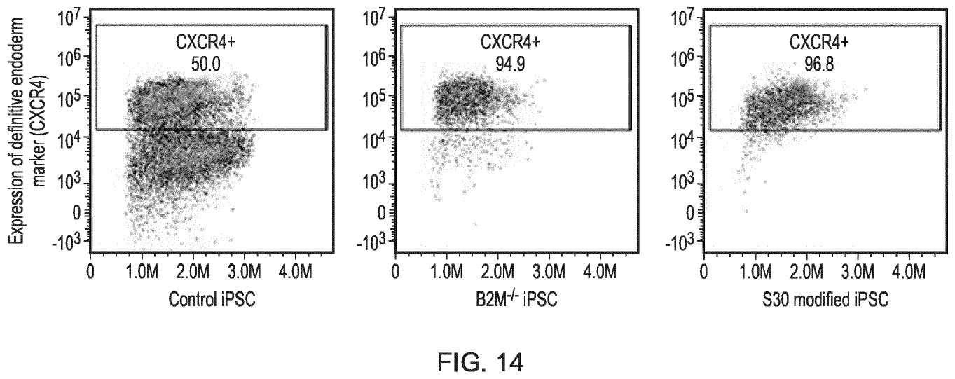

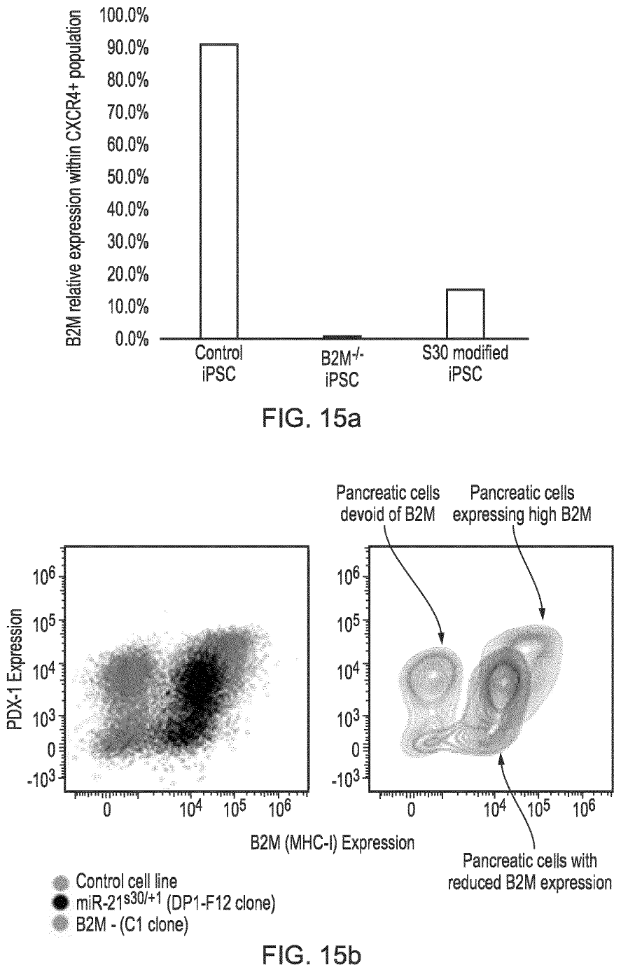

Pancreatic progenitor cells are generated from the control parental line (WT/WT) using a commercially available set of reagents and a validated methodology (Stem Cell Technologies; STEMDiff™ Pancreatic Progenitor Kit) that reproducibly generates progenitors via a four stage process of definitive endoderm, primitive gut tube, posterior foregut endoderm and pancreatic progenitors. Specified progenitors produced after fourteen days in continuous culture are isolated and assessed by flow cytometry for the level of staining for pancreatic progenitor markers (PDX-1, NKX6.1, co-stained for B2M). Results (see e.g. FIG. 14, FIG. 15A and FIG. 15B) indicate a significant proportion of the specified progenitors are positive for both pancreatic cell markers and the majority of PDX-1 positive cells also staining for B2M.

For solution 30, both heterozygous (WT/KI) and homozygous (KI/KI) clones are isolated, thus expressing the sRNA mono- and bi-allelically, respectively. GEiGS clones are assessed for the level of homogenous, quantitative and reproducible B2M silencing at the pancreatic progenitor stage of specification as the cells are differentiated from iPSC using the current method, with B2M expression being compared with the control parental line (WT/WT). The level of B2M silencing is conditional upon the progenitors remaining in the differentiated state, with this being lost when the progenitors de-differentiate, proliferate or become degenerative.

### Example 9—Differentiation of GEiGS Solution 30 Knock-In iPSC Line into Definitive Endoderm and Progenitors of the Pancreatic Lineage

Homozygous Solution 30 knock-in iPSC lines (miR-21^{S30/+1}) were differentiated into definitive endoderm and pancreatic lineage cells. A methodology for specifying pancreatic cells from pluripotent stem cells has been adopted from Balboa et al., Functional, metabolic and transcriptional maturation of human pancreatic islets derived from stem cells. Nat Biotechnol. 2022 July; 40(7):1042-1055. Doi: 10.1038/s41587-022-01219-z. Epub 2022 Mar. 3. PMID: 35241836; PMCID: PMC9287162.

There are many different protocols that have been published describing the specification of pancreatic lineage cells from iPSC and the skilled person is aware of other methods for specifying pancreatic cells from pluripotent stem cells. For example, see the references provided below. 1: DAmour K A, Agulnick A D, Eliazer S, Kelly O G, Kroon E, Baetge E E. Efficient differentiation of human embryonic stem cells to definitive endoderm. Nat Biotechnol. 2005 December; 23(12):1534-41. Doi: 10.1038/nbt1163. Epub 2005 Oct. 28. PMID: 16258519.
2: Schulz T C, Young H Y, Agulnick A D, Babin M J, Baetge E E, Bang A G, Bhoumik A, Cepa I, Cesario R M, Haakmeester C, Kadoya K, Kelly J R, Kerr J, Martinson L A, McLean A B, Moorman M A, Payne J K, Richardson M, Ross K G, Sherrer E S, Song X, Wilson A Z, Brandon E P, Green C E, Kroon E J, Kelly G, DAmour K A, Robins A J. A scalable system for production of functional pancreatic progenitors from human embryonic stem cells. PloS One. 2012; 7(5):e37004. Doi: 10.1371/journal.pone.0037004. Epub 2012 May 18. PMID: 22623968; PMCID: PMC3356395.
3: Kumar M, Jordan N, Melton D, Grapin-Botton A. Signals from lateral plate mesoderm instruct endoderm toward a pancreatic fate. Dev Biol. 2003 Jul. 1; 259(1):109-22. Doi: 10.1016/s0012-1606(03)00183-0. PMID: 12812792.
4: Melton D. The promise of stem cell-derived islet replacement therapy. Diabetologia. 2021 May; 64(5):1030-1036. Doi: 10.1007/s00125-020-05367-2. Epub 2021 January 16. PMID: 33454830; PMCID: PMC8012315.
5: Peterson Q P, Veres A, Chen L, Slama M Q, Kenty J H R, Hassoun S, Brown M R, Dou H, Duffy C D, Zhou Q, Matveyenko A V, Tyrberg B, Sörhede-Winzell M, Rorsman P, Melton D A. A method for the generation of human stem cell-derived alpha cells. Nat Commun. 2020 May 7; 11(1):2241. Doi: 10.1038/s41467-020-16049-3. PMID: 32382023; PMCID: PMC7205884.
6: Pagliuca F W, Millman J R, Gurtler M, Segel M, Van Dervort A, Ryu J H, Peterson Q P, Greiner D, Melton D A. Generation of functional human pancreatic β cells in vitro. Cell. 2014 Oct. 9; 159(2):428-39. Doi: 10.1016/j.cell.2014.09.040. PMID: 25303535; PMCID: PMC4617632.

The in vitro differentiation of pancreatic cells from iPSCs reproducibly progresses via a four stage process of definitive endoderm, primitive gut tube, posterior foregut endoderm and pancreatic progenitors. Specified progenitors produced after fourteen days in continuous culture, are isolated and assessed by flow cytometry for the level of staining for pancreatic progenitor markers (PDX-1, NKX6.1) as well as co-staining for B2M (MHC-I).

Solution 30 miRNA (hsa-mir-21) was demonstrated to be expressed at a low level in undifferentiated iPSCs and upregulated in differentiated cells, particularly pancreatic cells, compared with undifferentiated iPSC (see Example 8, FIG. 13). Accordingly, solution 30 is expressed at a higher level in differentiated cells, which is expected to lead to a decrease in B2M expression relative to the control (undifferentiated) iPSC line.

Heterozygous solution 30 knock-in clones were generated and differentiated into definitive endoderm and subsequent pancreatic progenitors. The iPSC clonal line (S30-DP1-F12) is a compound heterozygous genotyped with Solution 30 knocked into one allele and the other allele possessing a single base insertion which does not disrupt the transcription of the pre-miRNA or its scaffold secondary structure. The iPSC line (miR-21^{S30/+1}) was differentiated for 4 days using established methodology to specify definitive endoderm (detailed in Balboa D, et al 2022) and the proportion of cells expressing an early marker (CXCR4) assessed by flow cytometry.

The heterozygous knock-in line (iPSC miR-21^{S30/+1}) showed a high proportion of CXCR4 positive cells (approximately 97%), compared with both control (unmodified, 50%) and B2M^{-/-} iPSCs (95%) (FIG. 14). To confirm that the differentiating cells expressed reduced amounts of B2M, the population of CXCR4 positive cells were assessed for their B2M expression levels. The iPSC miR-21^{S30/+1} line showed an approximately 80% reduction in B2M expression compared with control (unmodified) iPSC cell line (FIG. 15A). As predicted B2M−/− iPSCs do not express the B2M protein.

The iPSC lines were differentiated for a further 7-10 days in culture, to a stage at which definitive endoderm cells express the pancreatic lineage marker, PDX-1. The pancreatic and duodenal homeobox-1 (PDX-1) marker gene is necessary for pancreatic development and Beta-cell maturation.

Results indicates a significant proportion of the specified progenitors at this later stage in development are positive for PDX-1 and the majority of PDX-1 positive cells also staining for B2M (FIG. 15B). The iPSC miR-21^{S30/+1} clonal line showed a high proportion of PDX-1 expressing cells which had markedly reduced B2M expression compared with the control (unmodified) iPSCs. The sRNA from Solution 30 was expressed mono-allelically.

These results demonstrate that B2M expression can be reduced using the GEiGS based solutions of the invention in a differentiation-dependent manner. Additionally, heterozygous knock-in of such solutions is sufficient to achieve significant and sustained knockdown. Any of SEQ ID NO: 1 or 18 (Solution 8), SEQ ID NO: 5 or 22 (Solution 20), SEQ ID NO: 9 or 26 (Solution 29) are expected to produce a similar effect to that observed with Solution 30.

### Example 10—Demonstrating B2M Silencing in Monocytes Specified in Culture from Clonal GEiGS-Modified iPSC

Solution 30 (see Tables 3 and 4) implemented as full GEiGS demonstrates that B2M silencing can be programmed to be deployed in other types of therapeutic cells including iPSC-derived monocytes. As in Example 3, the endogenous loci of the respective miRNA (hsa-mir-21) is modified. The homozygous (KI/KI) clones for Solution 30 were isolated, thus expressing the sRNA bi-allelically. GEiGS Solution 30 clones were assessed for the level of homogenous, quantitative and reproducible B2M silencing as the cells were differentiated into monocytes from iPSC. The hsa-mir-21 scaffold is expressed in a developmentally regulated and cell state specific manner in monocytes (Sheedy F J. Turning 21: Induction of miR-21 as a Key Switch in the Inflammatory Response. Front Immunol. 2015 Jan. 29; 6:19. Doi: 10.3389/fimmu.2015.00019. PMID: 25688245; PMCID: PMC4310327).

There are many published protocols for the generation of monocytes and macrophages from human pluripotent stem cells (for example Happle, C., Lachmann, N., Ackermann, M., Mirenska, A., Gohring, G., Thomay, K., Mucci, A., Hetzel, M., Glomb, T., Suzuki, T., et al. (2018). Pulmonary transplantation of human induced pluripotent stem cell-derived macrophages ameliorates pulmonary alveolar proteinosis. Am. J. Respir. Crit. Care Med. 198, 350-360; Lachmann, N., Ackermann, M., Frenzel, E., Liebhaber, S., Brennig, S., Happle, C., Hoffmann, D., Klimenkova, O., Luttge, D., Buchegger, T., et al. (2015). Large-scale hematopoietic differentiation of human induced pluripotent stem cells provides granulocytes or macrophages for cell replacement therapies. Stem Cell Reports 4, 282-296 and Zhang, H., Xue, C., Shah, R., Bermingham, K., Hinkle, C. C., Li, W., Rodrigues, A., Tabita-Martinez, J., Millar, J. S., Cuchel, M., et al. (2015). Functional analysis and transcriptomic profiling of iPSC derived macrophages and their application in modelling Mendelian disease. Circ. Res. 117, 17-28.

Monocytes were generated from the isogenic control parental line (parental control WT/WT line) and the homozygous (KI/KI) clone for Solution 30 (S30/S30 genotype) using a validated embryoid body-based protocol over a 3-4 week period. Undifferentiated iPSC were first differentiated into myeloid precursors and then monocytes generated using X-VIVO™ 15 Serum-free Hematopoietic Cell Medium (Lonza) supplemented with the cytokines IL-3 and Macrophage Colony-Stimulating Factor (M-CSF). All reagents were commercially available.

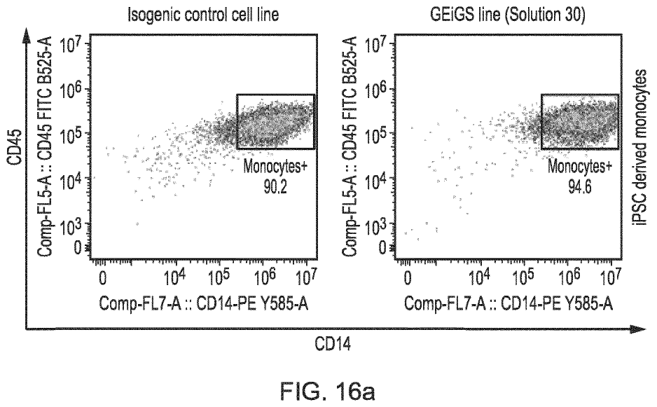

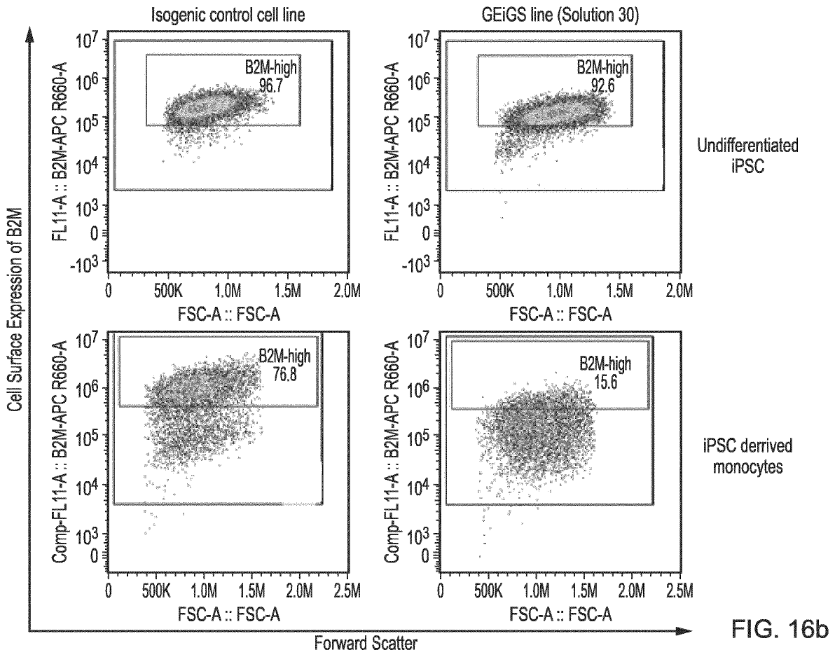

Monocytes were efficiently produced after approximately 3 weeks in continuous culture and were isolated and assessed by flow cytometry for the level of staining for the monocyte markers CD14 and CD45 markers (see FIG. 16a). Greater than 90% of the cells were double positive for the known monocyte markers CD14 and CD45. The iPSC lines (GEiGS Solution 30 and isogenic control) were also used to generate monocytes which were then stained for residual cell surface B2M expression using flow cytometry. When expanded as undifferentiated iPSC, greater than 93% of the cells for both the isogenic control and GEiGS Solution 30 clonal lines expressed B2M, whereas following differentiation into monocytes the GEiGS Solution 30 line showed a marked reduction in the number of cells with the same quantity of cell surface expressed B2M compared with the isogenic control line (16% cells with residual B2M compared with 77%) (see FIG. 16b).

### Example 11—Additional GEiGS Solutions

Further B2M GEiGS solutions were designed as set out in Example 1 and are detailed in Table 6. As before, pipeline inputs were: the mRNA sequence of B2M (GenBank Accession: NM_004048.4), the human genome reference sequence (GRCh38), miRNA annotations (miRbase v22) and cell type specific miRNA quantifications.

Expression of miRNAs in human iPSCs and ESCs was quantified using publicly available next-generation sequencing (NGS) datasets: ENCODE accession ENCSR958UOC, ENCSR430YFL.

The GEiGS® solution designs were comprised of a modified sequence of an endogenous pre-miRNA (hair-in)—also referred to as the endogenous miRNA scaffold—modified to encode a novel silencing RNAi trigger instead of its native guide strand. The novel GEiGS® silencing RNA matched the 1B2M mRNA by sequence complementarity and was predicted to lead to the silencing of 1B2M through the RNA interference pathway.

Extended solutions are comprised of a GEiGS solution as designed computationally, plus 50-150 bases or 40 bp of genomic sequence flanking on both sides of the endogenous pre-miRNA that the respective GEiGS solution was based on. This is included to avoid omitting important sequences that act as a signal for the enzyme Drosha.

The B2M GEiGS solutions detailed in Table 6 are tested for silencing activity via the plasmid-based ectopic expression assay detailed in Example 2. As before, a broad spectrum of 1B2M silencing is observed, ranging from 5%-99%. The most effective solutions showed strong silencing activity.

TABLE 6
Sequences of computationally-designed B2M-targeting
RNAi trigger sequences and GEiGS solutions

| GEiGS | | GEiGS mature sRNA | GEiGS solution sequence |
|---|---|---|---|
| Solution | miRNA | sequence (guide) | (pre-miRNA hairpin) |
| ID | scaffold | [SEQ ID NO: #] | [SEQ ID NO: #] |
| Sol-1 | hsa-mir- | TAAACCTGAATCTTTGG | CTCCCCTCTACTCCAAATTTTCAGGTCTACCCGGAG |
| | 320a | AGTAC [64] | TCGGGtaaacctgaatctttggagtacAAAAGGATG |
| | | | [128] |
| 2 | hsa-mir- | TAAACCTGAATCTTTG | TCTCAGGCTGTGGTACTCCAAAGATTCAGGTT |
| | 520f | GAGTAC [65] | TTCTGTGGTCAGAAAGAAAAGtaaacctgaatctt |
| | | | tggagtacTACCGTTTGGGA [129] |
| 3 | hsa-mir- | TAAACCTGAATCTTTG | ACGAATGGCTATGCACTGTACTCCAAAGAGA |
| | 652 | GAGTAC [66] | GAGTCAGGTTTACACATAGACTATAATTGtaaa |
| | | | cctgaatctttggagtacAGTGCACAACCTACAC |
| | | | [130] |
| 4 | hsa-mir- | TTCAGTAAGTCAACTT | CCTTGGCCATGTttcagtaagtcaacttcaatgtcCTT |
| | 106a | CAATGTC [67] | TTTGAGATGACTTGAAATTGACTTACTGCAAA |
| | | | CATTACCATGG [131] |
| 5 | hsa-mir- | TTTTTCAATTCTCTCTC | GCTGCTGGGGAGAGCGATTGAAAAATAGTGC |
| | 1180 | CATTC [68] | TCCTGGTTGtttttcaattctctctccattcCGGCGGC |
| | | | [132] |
| 6 | hsa-mir- | TTTTTCAATTCTCTCTC | TTGAGGCCTTAAAGTACTGtttttcaattctctctcca |
| | 15b | CATTC [69] | ttcTGCTACAGTCAAGAGACTGGAGAGTGATT |
| | | | TGAAAACTAGAAATTTAAGGAAATTCAT [133] |
| 7 | hsa-mir- | TTCTTCAGTAAGTCAA | GAGCTGCTTGCCTCCCCCCGTTttcttcagtaagtc |
| | 182 | CTTCAATG [70] | aacttcaatgGGTGAGGTAACAGGATCCCATTGT |
| | | | TGACGACCTGAAGACTATGGGGCGAGGACTC |
| | | | AGCCGGCAC [134] |
| 9 | hsa-mir- | TTCAGTGTAGTACAA | GGCCGGCCTATCCTTGTACGACACGGAATTGC |
| | 23a | GAGATAG [71] | TTCCTGTCACAAttcagtgtagtacaagagatagACC |
| | | | GACC [135] |
| 10 | hsa-mir- | TTCAGTAAGTCAACTT | CCGGGACCCAGttcagtaagtcaacttcaatgtGTGT |
| | 26b | CAATGT [72] | GCTGTCCACATTGAAGTCCACTTACTGACTCG |
| | | | GGGACCGG [136] |
| 13 | hsa-mir- | TTTCAATTCTCTCTCC | CTCCCCTCAGAATGGAGTTAGAATTGCAACCC |
| | 320a | ATTCTT [73] | GGAGTCGGGtttcaattctctctccattcttAAAAGGA |
| | | | TG [137] |
| 14 | hsa-mir- | TTCAGTAAGTCAACTT | TGTTTTGAGCGGGGGttcagtaagtcaacttcaatgt |
| | 335 | CAATGTC [74] | cTTGTCATAAACACATTGAAATTGACTTACCGA |
| | | | ACCTCCTCTCATTTGCTATATTCA [138] |
| 15 | hsa-mir- | TTTCAATTCTCTCTCC | GGAGCtttcaattctctctccattcttTTTATAATTTCA |
| | 361 | ATTCTT [75] | AAAAAACAATGAAGTGTGAGAATTGATTGCT |
| | | | TC [139] |
| 16 | hsa-mir- | TTCTTCAGTAAGTCAA | TCATGCTGTGGCTGAACTTGACTAACTGAAGT |
| | 518b | CTTCAA [76] | CAATTGTCTGAAAGAAAAttcttcagtaagtcaactt |
| | | | caaTTACGGTTTGA [140] |
| 17 | hsa-mir- | TTTCAATTCTCTCTCC | TCTCAGGCTGTGAAGAATGGAGAGAGAATTG |
| | 520f | ATTCTT [77] | AATCTGTGGTCAGAAAGAAAAGtttcaattctctct |
| | | | ccattcttTACCGTTTGGGA [141] |
| 18 | hsa-mir- | TTCAGTGTAGTACAA | ACGAATGGCTATGCACTCTATCTCTTGTAGAG |
| | 652 | GAGATAG [78] | AGTACACTGAACACATAGACTATAATTGttcagt |
| | | | gtagtacaagagatagAGTGCACAACCTACAC |
| | | | [142] |
| 22 | hsa-mir- | TTAACTATCTTAACAA | GCTGCTGCGCTTGTCAAATAGTTAATAGTGCT |
| | 1180 | GCTTTG [79] | CCTGGTTGttaactatcttaacaagctttgCGGCGGC |
| | | | [143] |
| 23 | hsa-mir- | TAACAAATTTCCAATA | CATCAAGACCCAGCTGAGTCACTGTCACTGCC |
| | 1307 | ATCCTGT [80] | TACCAAACTGGATTATAGATATATTGTCACGT |
| | | | CTGTGTTGCCAATCGtaacaaatttccaataatcctgt |
| | | | TAGATAGGCGGTCATGCATACGAATTTTCAGC |
| | | | TCTTGTTCTGGTGAC [144] |
| 24 | hsa-mir- | TAACATTATTATAACC | TTGAGGCCTTAAAGTACTGtaacattattataaccct |
| | 15b | CTACAT [81] | acatTGCTACAGTCAAGAATCTAGGGTTTTATT |
| | | | AATGTTCTAGAAATTTAAGGAAATTCAT [145] |
| 25 | hsa-mir- | TATCAATATTAAAAA | GAGCTGCTTGCCTCCCCCCGTTtatcaatattaaaa |
| | 182 | GCAAGCAAG [82] | agcaagcaagGGTGAGGTAACAGGATCCCTTGG |
| | | | CTTTTGACTATTGATCTATGGGGCGAGGACTC |
| | | | AGCCGGCAC [146] |
| 28 | hsa-mir- | TTCATTCATTATAACA | AAAGATCCTCAGACAATCCATGTGCTTCTCTT |
| | 205 | AATTTC [83] | GttcattcattataacaaatttcTCTCATACCCAACGA |
| | | | AATTTGATATAATGAATGTTCAGGAGGCATG |
| | | | GAGCTGACA [147] |
| 31 | hsa-mir- | TTAACATTATTATAAC | GGCTGAGCCGCATGTAGGGTTATATAATGATT |
| | 22 | CCTACA [84] | AATATGTCCTGACCCAGCTAttaacattattataacc |
| | | | ctacaTGCCCTCTGCC [148] |
| 32 | hsa-mir- | TAACATTATTATAACC | TGAACATCCAGGTCTGGGGCATAATTAGGGT |
| | 221 | CTACATT [85] | TTTAATAATGTATTTTAGTGTTCGTTAGGCAAC |
| | | | taacattattataaccctacattAGGCTACCTGGAAAC |
| | | | ATGTTCTC [149] |
| 33 | hsa-mir- | TGTTAACATTATTATA | GCTGCTGGAAGGTGTAGGTACCCTCAATTAG |
| | 222 | ACCCTACA [86] | GGTTATAATAAGTTAAATCCTGTCTTTCGTAAT |
| | | | CAGCtgttaacattattataaccctacaTGATGGCATC |
| | | | TTCTAGCT [150] |
| 34 | hsa-mir- | TATAAAGAAGATCAT | GGCCGGCCATGGCATGATCGTCTTGATATTGC |
| | 23a | GTCCATG [87] | TTCCTGTCACAAtataaagaagatcatgtccatgACC |
| | | | GACC [151] |
| 37 | hsa-mir- | TTTATGATTTATTTAA | GGGCAGTCTTTGCTACtttatgatttatttaacttgtgg |
| | 30e | CTTGTGGA [88] | aGTAAGGTGTTCAGAGGTCCACAAGTTATAAA |
| | | | TCATAAAGCGGCAGGCTGCCA [152] |
| 38 | hsa-mir- | TTATTATAACCCTACA | CTCCCCTCCAAAATGTATTGTTATAACAACCCG |
| | 320a | TTTTGT [89] | GAGTCGGGttattataaccctacattttgtAAAAGGA |
| | | | TG [153] |
| 39 | hsa-mir- | TAACATTATTATAACC | TGTTTTGAGCGGGGGtaacattattataaccctacat |
| | 335 | CTACATT [90] | tTTGTCATAAACATGTAGGGATATAATAATCTT |
| | | | ACCTCCTCTCATTTGCTATATTCA [154] |
| 40 | hsa-mir- | TTATTATAACCCTACA | GGAGCttattataaccctacattttgtTTTATAATTTC |
| | 361 | TTTTGT [91] | AAAAAACCAAATATATGTGGTTATAATTTGCT |
| | | | TC [155] |
| 42 | hsa-mir- | TAACTTATGCACGCTT | ATAAAGGAAGTTAGGATAGTTAAGAGGCAGA |
| | 423 | AACTATC [92] | AGTATATTTCTATTTTCCAAAtaacttatgcacgctt |
| | | | aactatcCTTGCTTCCTAACCCGCGC [156] |
| 44 | hsa-mir- | TAACATTATTATAACC | TCTCAGCCTGTGACGTAGGGTTATAAAAATGT |
| | 519c | CTACAT [93] | TACTGTTGTCTGAAAGAAAAGtaacattattataa |
| | | | ccctacatTACAGTTTGAGA [157] |
| 45 | hsa-mir- | TTTATGATTTATTTAA | TCTCAGGCTGTGCACAAGTTAAATAAATCATA |
| | 520f | CTTGTG [94] | ATCTGTGGTCAGAAAGAAAAGtttatgatttattta |
| | | | acttgtgTACCGTTTGGGA [158] |
| 46 | hsa-mir- | TTCATTATAACAAATT | ACGAATGGCTATGCACTATTGGAAATTTGGA |
| | 652 | TCCAAT [95] | GAGTATAATGAACACATAGACTATAATTGttca |
| | | | ttataacaaatttccaatAGTGCACAACCTACAC |
| | | | [159] |
| 48 | hsa-mir- | TTTTATGATTTATTTA | CGGGCCCCGGGCGGGCGACAAGGATAAATA |
| | 92b | ACTTGT [96] | ACATCATAAAATTGTTTTTTCCCCCGCCAAtttta |
| | | | tgatttatttaacttgtGGCCCCCCCGGCCC [160] |
| 152 | hsa-mir- | TTAACTTATGCACGCT | CCACTAGTAAAGCGTGCGAGAGTTAACTTTGA |
| | 302a | TAACTAT [103] | AACTAAAGAAGTTAACTTATGCACGCTTAACT |
| | | | ATTGG [167] |
| 153 | hsa-mir- | TAACAAATTTCCAATA | CCACCAGGTTTATTGGAATTTTGTTACTTTGAA |
| | 302a | ATCCTGT [104] | ACTAAAGAAGTAACAAATTTCCAATAATCCTG |
| | | | TTGG [168] |
| 154 | hsa-mir- | TTAACTTATGCACGCT | GCTCACTTCAAGTATAGCGTGCATAAGTTATC |
| | 302b | TAACTAT [105] | TGTGACTTTAAAAGTTAACTTATGCACGCTTA |
| | | | ACTATGAGT [169] |
| 155 | hsa-mir- | TAACAAATTTCCAATA | GCTCCCTTCAAGGTATATTGGAAATTTGTTTCT |
| | 302b | ATCCTGT [106] | GTGACTTTAAAAGTAACAAATTTCCAATAATC |
| | | | CTGTGAGT [170] |
| 156 | hsa-mir- | TTAACTTATGCACGCT | CCTATAGTCTGGCGTGCATAAGTCAACTGTGT |
| | 302c | TAACTAT [107] | GAAACAAAAGTTAACTTATGCACGCTTAACTA |
| | | | TAGG [171] |
| 157 | hsa-mir- | TAACAAATTTCCAATA | CCTACAGGTCTATTGGAAATTTGCTACTGTGT |
| | 302c | ATCCTGT [108] | GAAACAAAAGTAACAAATTTCCAATAATCCTG |
| | | | TAGG [172] |
| 158 | hsa-mir- | TACAAGAGATAGAAA | CCACACTGTTCTTTCTATGTCTTGTACTTTGAA |
| | 302a | GACCAGTC [109] | ACTAAAGAAGTACAAGAGATAGAAAGACCAG |
| | | | TCTGG [173] |
| 159 | hsa-mir- | TACAAGAGATAGAAA | GCTCGCTTCCCTGTACTTTCTATCTCTTGTTCT |
| | 302b | GACCAGTC [110] | GTGACTTTAAAAGTACAAGAGATAGAAAGAC |
| | | | CAGTCGAGT [174] |
| 160 | hsa-mir- | TACAAGAGATAGAAA | CCTGACTGTGCTTTCTATCTCTTCTACTGTGTG |
| | 302c | GACCAGTC [111] | AAACAAAAGTACAAGAGATAGAAAGACCAGT |
| | | | CAGG [175] |
| 161 | hsa-mir- | TTAACTTATGCACGCT | CCCAGTTCAAGCGTCTTTACGTTAACCGGACC |
| | 375 | TAACTA [112] | TGAGCGTTTAACTTATGCACGCTTAACTAGGC |
| | | | [176] |
| 162 | hsa-mir- | TAACAAATTTCCAATA | CCAAGGACTTATTGCCTATCTGTTACCGGACC |
| | 375 | ATCCTG [113] | TGAGCGTTAACAAATTTCCAATAATCCTGGGC |
| | | | [177] |
| 163 | hsa-mir- | TACAAGAGATAGAAA | CCTCTGGATCTTTCCCACTATTGTACCGGACCT |
| | 375 | GACCAGT [114] | GAGCGTTACAAGAGATAGAAAGACCAGTGGC |
| | | | [178] |
| 309 | hsa-let- | TTGACTTTCCATTCTC | CGGGGTTGACTTTCCATTCTCTGCTGGTCAGG |
| | 7b | TGCTGG [115] | GCAGTGATGTTGCCCCTCGGCAAATCCAGCA |
| | | | GAGAATGGAAAGTCACCCTG [179] |
| 310 | hsa-let- | TTGACTTTCCATTCTC | AGGCTTGACTTTCCATTCTCTGCTGGTGAGGG |
| | 7g | TGCTGG [116] | TCTAGGATACCACCCGGTACCGGAGATCCAG |
| | | | CAGAGAATCGAAAGTCAGCCA [180] |
| 311 | hsa- | TTGACTTTCCATTCTC | AGGATTCTGCTCATGCCAGGGTTGACTTTCCA |
| | mir-98 | TGCTGG [117] | TTCTCTGCTGGGTGGGGTAGGGATATTAGGC |
| | | | CCCAATTAGAAGATCCATCAGAGAATGGAAA |
| | | | GTCTCCCTGGTGTGTGGCATATTCA [181] |
| 312 | hsa- | GGCGTCTCGGTGGCA | AGGATTCTGCTCATGCCAGGGGGCGTCTCGG |
| | mir-98 | CGACTTGT [118] | TGGCACGACTTGGTGGGGTAGGGATATTAGG |
| | | | CCCCAATTAGAAGACCAATTCGTGCCACCGAG |
| | | | ATGACCCTGGTGTGTGGCATATTCA [182] |
| 313 | hsa-let- | TAACAAATTTCCAATA | CGGGGTAACAAATTTCCAATAATCCTGTCAGG |
| | 7b | ATCCTGT [119] | GCAGTGATGTTGCCCCTCGGAAGCTCAGGATT |
| | | | ATTGGAAATTTGTTCCCTG [183] |
| 314 | hsa-let- | TAACAAATTTCCAATA | AGGCTAACAAATTTCCAATAATCCTGTGAGGG |
| | 7g | ATCCTGT [120] | TCTATGATACCACCCGGTACAGGAGATCAGG |
| | | | ATTATTGGCAATTTGTTGCCA [184] |
| 315 | hsa- | TAACAAATTTCCAATA | AGGATTCTGCTCATGCCAGGGTAACAAATTTC |
| | mir-98 | ATCCTGT [121] | CAATAATCCTGGTGGGGTAGGGATATTAGGC |
| | | | CCCAATTAGAAGATCAGTATTATTGGAAATTT |
| | | | GTCCCCTGGTGTGTGGCATATTCA [185] |
| 316 | hsa-let- | TTAACTTATGCACGCT | CGGGGTTAACTTATGCACGCTTAACTATCAGG |
| | 7b | TAACTAT [122] | GCAGTGATGTTGCCCCACGGAAGATTAGTTA |
| | | | AGCGTGCATAAGTTACCCTG [186] |
| 317 | hsa-let- | TTAACTTATGCACGCT | AGGCTTAACTTATGCACGCTTAACTATGAGGG |
| | 7g | TAACTAT [123] | TCTATGATACCACCCGGTACAGGAGATTAGTT |
| | | | AAGCGTGATTAAGTTAGCCA [187] |
| 318 | hsa- | TTAACTTATGCACGCT | AGGATTCTGCTCATGCCAGGGTTAACTTATGC |
| | mir-98 | TAACTAT [124] | ACGCTTAACTAGTGGGGTAGGGATATTAGGC |
| | | | CCCAATTAGAAGCTTAGCTAAGCGTGCATAAG |
| | | | TTTCCCTGGTGTGTGGCATATTCA [188] |
| 319 | hsa-let- | TACAAGAGATAGAAA | CGGGGTACAAGAGATAGAAAGACCAGTTCAG |
| | 7b | GACCAGTC [125] | GGCAGTGATGTTGCCCCTCGGAAGATACTGG |
| | | | TCTTTCTATCTCTTGTCCCTG [189] |
| 320 | hsa-let- | TACAAGAGATAGAAA | AGGCTACAAGAGATAGAAAGACCAGTTGAGG |
| | 7g | GACCAGTC [126] | GTCTATGATACCACCCGGTACAGGAGATACTG |
| | | | GTCTTTCTCTCTCTTGTGCCA [190] |
| 321 | hsa- | TACAAGAGATAGAAA | AGGATTCTGCTCATGCCAGGGTACAAGAGAT |
| | mir-98 | GACCAGTC [127] | AGAAAGACCAGTGTGGGGTAGGGATATTAG |
| | | | GCCCCAATTAGAAGATACTTGTCTTTCTATCTC |
| | | | TTGCCCCTGGTGTGTGGCATATTCA [191] |

### Example 12—Assessment of Effects of Homology Arm Length on Efficiency of Homology Directed Repair

The concentration and type of HDR (homology directed repair) templates with different lengths of homology arms was varied to assess if this impacts the efficiency of editing miRNA loci for the insertion of GEiGS templates via homology directed repair. ssDNA (single stranded DNA) may increase accurate editing efficiency compared to dsDNA (double stranded DNA) templates by promoting duplex formation and therefore HDR, as well as reducing the risk of repair by end-joining mechanisms that would lead to undesired editing outcomes [Skarnes W C, Pellegrino E, McDonough J A. Improving homology-directed repair efficiency in human stem cells. Methods. 2019 Jul. 15; 164-165:18-28. doi: 10.1016/j.ymeth.2019.06.016. Epub 2019 Jun. 16. PMID: 31216442]. Furthermore, shorter homology may allow a greater amount and concentration of donor to be transfected into cells and therefore promote efficient HDR editing.

iPSCs were transfected with Cas9-guide RNA complexes at 4 μM (micromolar) and HDR templates at varying concentrations (μM). Different forms of HDR templates were tested: short ssDNA with 40 bp-long right and left homology arms with modified ends (137 bp total length, purchased with AltR HDR modfications from Integrated DNA Technologies) were compared to long ssDNA with 350 bp-long right and left homology arms (632 bp total length, purchased from Integrated DNA Technologies) or short HDR blocks (dsDNA purchased with from Integrated DNA Technologies with modified ends) with 150- or 350 bp-long right and left homology arms (137 or 632 bp total length). The efficiency of HDR was assessed by performing next generation sequencing on the amplified miRNA locus of interest (the miR-21 locus) and analysed using an allele quantification software (CRISPresso2)[Clement K, Rees H, Canver M C, Gehrke J M, Farouni R, Hsu J Y, Cole M A, Liu D R, Joung J K, Bauer D E, Pinello L. CRISPResso2 provides accurate and rapid genome editing sequence analysis. Nat Biotechnol. 2019 March; 37(3):224-226. doi: 10.1038/s41587-019-0032-3. PubMed PMID: 30809026] (FIG. 17). FIG. 17 shows that flanking genomic sequences (homology arms) of length 40 base pairs show superior effects for the efficiency of HDR (% HDR) and in a dose dependent manner, compared to flanking genomic sequences of length 350 bp or length 150-350 bp which are only marginally better to no donor template alone.

At similar concentrations (0.357 or 2 μM) short ssDNA resulted in higher editing efficiency (% HDR) compared to long ssDNA or short dsDNA HDR blocks. The greatest editing efficiency was observed after increasing the concentration of short ssDNA to 8 μM. Higher concentrations of long ssDNA or short HDR blocks could not be tested due to physical constraints in synthesising these templates.

### Example 13—Ability of GEiGS Solution-29 to Buffer Against the Induced Expression of B2M Following Exposure of Cells to an Inflammatory Cytokine

GEiGS is suitable for the generation of hypoimmunogenic cells as it has the potential to provide stable and tuneable knock-down of B2M, and therefore partial silencing of MHC-I expression rather than completely knocking it out (KO). MHC-I KO is useful for effective evasion of the recipients adaptive immunity, but exposes the therapeutic cells to rejection by cells of the innate immune system.

The cell or composition according to the present invention may be for use in a method of treating an autoimmune disease or cancer in a subject, wherein the method comprises administering the cell or composition in a therapeutic amount to the subject. The autoimmune disease may be any autoimmune disease, preferably Type I diabetes. For treating diabetes, the cells of the invention may be transplanted to a diabetic patient as differentiated pancreatic beta islet cells from stem cells.

Autoimmune disorders or cancer are typified by chronic inflammation in patients. In Type 1 diabetes and cancer therefore, the cells or cellular compositions transplanted as interventions will be exposed to inflammatory stimuli. Many different factors can affect the basal and inducible expression of MHC-I on the cell surface. These include factors to be found in diseased, infected and inflamed tissues, such as cytokines, growth factors [interferons (IFNs), tumour necrosis factor (TNF), and type II interferon (IFN-y)]. IFNs have a capacity to activate the expression of major and minor HLA genes, components of the MHC-I complex.

B2M KO would prevent MHC-I upregulation in therapeutic cells in response to inflammatory cytokines. For GEiGS to be an effective alternative to B2M KO for maintaining hypoimmune cells even when exposed to inflammation, its silencing effect needs to be demonstrated as sufficiently dynamic to maintain both stable and tunable gene silencing.

Various media conditions were tested to assess their effects on B2M expression in iPSC. Interferon-gamma (IFN-y or INF-g) was included at a concentration of 20 ng/ml, a supra-physiological concentration used to induce IFN-response genes in experimental tissue culture. Exposure of control (unmodified) iPSC to IFN-y for 72 hours (WT+INFg) led to a more than 6 times increase in the relative expression level of B2M as analyzed by flow cytometry using anti-B2M antibodies, compared with unstimulated iPSC alone (WT).

Unexpectedly, while the homozygous (KI/KI) Solution 29 clonal line expressing the B2M sRNA bi-allelically displayed an elevated expression of cell surface B2M in the presence of INF-y (S29+ INFg), this increase was much reduced (7 fold lower) compared with that for the parental line exposed to IFN-y for 72 hours (WT+INFg), suggesting that GEiGS has a profound buffering effect in preventing the upregulation of MHC-I on cells in response to an inflammatory cytokine (FIG. 18)). The relative fold change in B2M cell surface expression (MHC-I cell surface expression) between S29 and S29+INF-γ (approximately 10 times) is approximately the same as the relative fold change between WT and WT+INF-γ (approximately 14 times). In this experiment, the cell surface level of B2M is indicative of the cell surface level of MHC-I since MHC-I complex is a heterodimer made up of a hypervariable HLA protein and beta-2 microglobulin (B2M).

This demonstrates that GEiGS solutions of the invention targeting B2M can also reduce B2M (and therefore MHC-I) levels in chronic inflammatory conditions. This indicates that such solutions could be of therapeutic benefit to patients, even those suffering from chronic inflammation diseases. Additionally, this example illustrates that the effects of the inhibitory RNA is not limited by the copy number of target transcripts in the cell.

Any of SEQ ID NO: 1 or 18 (Solution 8), SEQ ID NO: 5 or 22 (Solution 20), SEQ ID NO: 9 or 26 (Solution 29) are expected to produce a similar effect to that observed with Solution 30,

### Example 14—Demonstration of the Ability of GEiGS to Silence One or More Target Genes in Cells when Distinct Solutions were Ectopically Expressed from a Single Construct

Constructs were used to ectopically express Solutions targeting B2M and eGFP (Solution 179) or two distinct B2M-silencing Solutions as a concatemer (Solution 29 and Solution 43, or two copies of Solution 29) under a constitutive promoter, via transfection into iPSC, as described in Example 2. The plasmid map for expression of one or more Solutions is shown in FIG. 19a.

Two separate iPSC lines were used; a characterised, clonal cell line in which one copy of the sox-2 gene has been modified to incorporate mEGFP so as to not disrupt the protein coding sequence (Allen Institute, AICS-0074-026:WTC-mEGFP-sox2-cl26) and the parental control iPSC line used to generate this tagged line (WTC, Coriell Institute for Medical Research, cell line biorepository, sample GM25256). Cells were collected three days after transfection and analysed by flow cytometry as previously described (Example 2).

B2M and eGFP

GEiGS Solution 101 consists of a miR-30 scaffold modified to incorporate eGFP trigger sequences (miR-30_GFP_Leb_130) in order to target GFP alone and with extended homology arms of 130 bp represented of the adjacent scaffold sequences (Fellman, et al.). The miR-30 GFP sequences are extracted from the publication Lebbink R J, Lowe M, Chan T, Khine H, Wang X, McManus M T. Polymerase II promoter strength determines efficacy of microRNA adapted shRNAs. PLoS One. 2011; 6(10):e26213. doi: 10.1371/journal.pone.0026213. Epub 2011 Oct. 21. PMID: 22031824; PMCID: PMC3198731. Solution 179 consists of a polycistronic sequence consisting of Solution 29 (targeting B2M) and miR-30_eGFP_Leb_130 (targeting GFP) (two distinct modified miRNA scaffolds (miRNA-20a and miRNA-30)).

Compared with a negative control vector transfection, Solution 101 demonstrated an efficient GFP silencing without affecting the level of expression of B2M in the undifferentiated iPSC (FIG. 19b). Solution 179, consisting of two distinct modified miRNA scaffolds (miRNA-20a and miRNA-30), was effective in silencing both B2M and eGFP with apparent equal efficiency (FIG. 19b).

This demonstrates that multiple GEiGS solutions expressed from the same construct can be effective at silencing multiple targets.

### Two Separate Solutions Targeting B2M

GEiGS Solution 177 was made consisting of a Solution 29 (based on modified miRNA-20a) combined with Solution 43 (based on modified miR-518b), distinct Solutions with diverse sequences targeting the B2M transcript (triggers). Solution 178 was made consisting of two copies of the Solution 29 (targeting B2M).

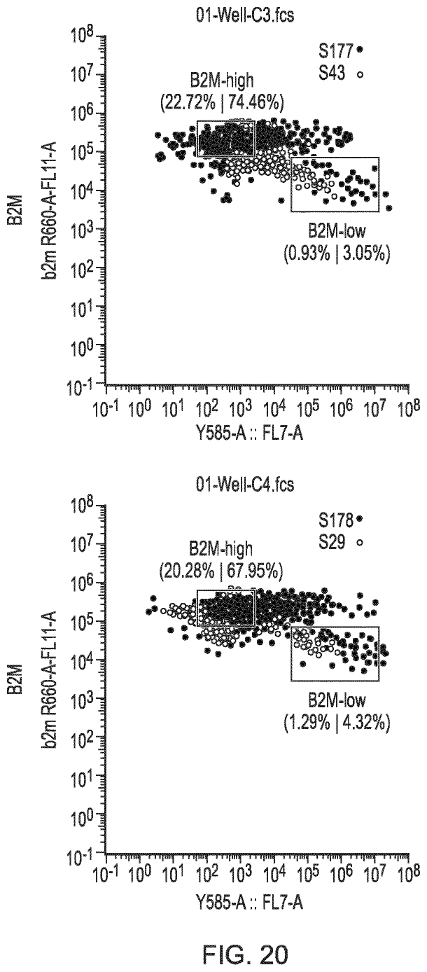

Compared with a negative control vector transfection, Solution 177 (2 different solutions targeting B2M) appeared effective in silencing B2M when compared to the Solution 43 alone, indicating a minor additive effect and that the two distinct Solutions do not interfere in each others' ability to silence B2M (FIG. 20). Whereas Solution 178 which possesses two copies of Solution 29 and the same trigger sequences, does not have an additive effect in silencing B2M compared with Solution 29 alone (FIG. 20).

### Example 15—Analysis of Potential Off-Target Effects Caused by GEiGS Demonstrates that GEiGS is Very Specific, Showing No Off-Target Effects

Systematic unbiased whole transcriptome analysis of Solution 29 (S29) GEiGS lines and corresponding controls was performed to assess whether GEiGS-mediated silencing causes off-target effects.

Isogenic GEiGS modified iPSC cells and control iPSC cell lines were generated from the same parental iPSC line and RNA-seq was used to measure gene expression. A differential gene expression (DGE) analysis was performed using the parental line as reference to detect transcriptional changes genome-wide in an unbiased way. This approach detects both direct changes in gene expression caused by GEiGS (specific target silencing, potential silencing of unintended targets, and potential upregulation of the cognate target of the redirected miRNA) as well as indirect changes caused by knock-on effects from the direct changes in gene expression. RNA-seq and DEG analysis were performed using standard techniques.

The GEiGS lines analysed were homozygous for solution 29 knock-in (S29 KI/KI), which is a B2M-targeting GEiGS solution based on MIR20A as scaffold (see Example 3 for details of implementation of GEiGS for Solution 29). The following control lines were also generated:
(i) B2M homozygous KO (B2M KO/KO) generated by CRISPR KO, to detect transcriptional changes caused specifically by B2M silencing; and
(ii) hsa-mir-20a homozygous KO (hsa-mir-20a KO/KO), to detect changes in gene expression caused by the loss of activity of the miRNA gene used hijacked as scaffold for solution 29.

Each experimental sample included at least two biological replicates (independent clones with the same genotype) in two technical replicates (independent cultures of the same clone). DGE analysis was performed using the R package edgeR, using false discovery rate (FDR) 0.05 as a statistical significance threshold, with no effect size cutoff.

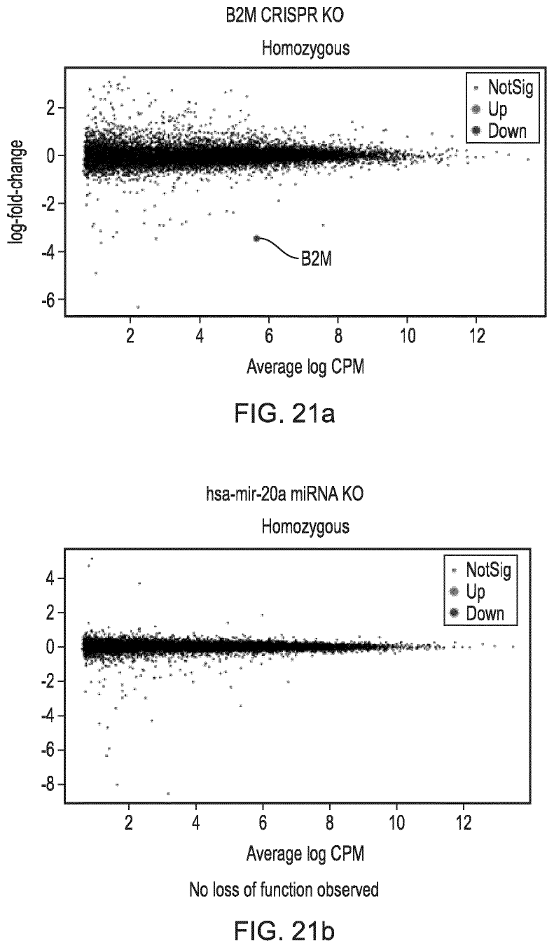

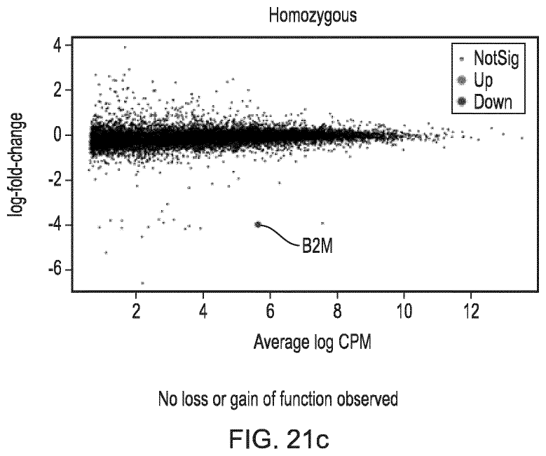

Results are shown in FIG. 21a-21c. When comparing B2M KO/KO to the parental cell line, it can be seen that the CRISPR KO of B2M led to a very specific and significant reduction of mRNA levels transcribed from the B2M gene (91% down-regulation); B2M is the only differentially expressed gene (FIG. 21a). No other changes in gene expression were detected indicating loss of MHC-I expression in a steady state culture of iPSC has no consequence on the physiology of the cell (FIG. 21a).

In the KO of hsa-mir-20a, no changes in expression were caused by the loss of the edited miRNA, providing experimental evidence for the functional redundancy in the miRNA network that is leveraged by GEiGS (FIG. 21b).

In the GEiGS modified cells in which Solution 29 had been introduced into the endogenous hsa-mir-20a locus, again there was very specific and significant reduction of mRNA levels transcribed from the B2M gene compared to the parental cell line; B2M is again the only differentially expressed gene (FIG. 21c). This shows that GEiGS is very specific, only silencing the intended target and no other unintended targets are silenced.

Notably, off-target effects previously associated with silencing of unintended targets when using siRNA or shRNA are not detected. This further supports the application of GEiGS for the highly specific silencing of target genes, avoiding off-target effects such as silencing of unintended targets as well as potential loss-of-function effects following redirection of miRNA genes used as scaffolds.

### Example 16—Demonstrating B2M Silencing in Myeloid Cells Results in Loss of Cell Surface MHC-I (as Evidenced by Loss of HLA-A/B/C Expression Using Antibody Labelling)

A number of the Solutions identified for silencing B2M (e.g. Solution 30) were implemented as full GEiGS, i.e. by editing the endogenous loci of the respective miRNA scaffolds in iPSC and differentiating the stem cells into myeloid cells.

GEiGS was implemented using gRNAs for individual Solution 30, as obtained from Integrated DNA Technologies (IDT) as synthetic sgRNAs. Cas9 protein is obtained from IDT. Isolated iPSC were electroporated using the Lonza 4D-Nucleofector device (4D-25 Nucleofector Core Unit: Lonza, AAF-1002B; 4D-Nucleofector X Unit: AAF-1002X) (Buffer P3, device setting CM-137) and then contacted with the sgRNA/Cas9 ribo-nucleoprotein complexes (RNP) and the appropriate dsDNA HDR template either produced internally or made commercially (IDT or Genewiz).

After electroporation, clone selection and characterisation, clonal iPSC cell lines with either homozygous knock-out of the miRNA-21 gene or homozygous knock-in of the Solution 30 design into the miR-21 locus, were differentiated into monocytes and then monocyte-derived macrophages in macrophage medium (DMEM high glucose supplemented with 10% FBS [Gibco], 2mMGlutaMAX, 100 U/ml penicillin, 100 μg/ml streptomycin, and M-CSF 100 ng/ml [Peprotech]) and the medium changed every 2-3 days by adding half the volume of medium with 1× cytokines into each well.

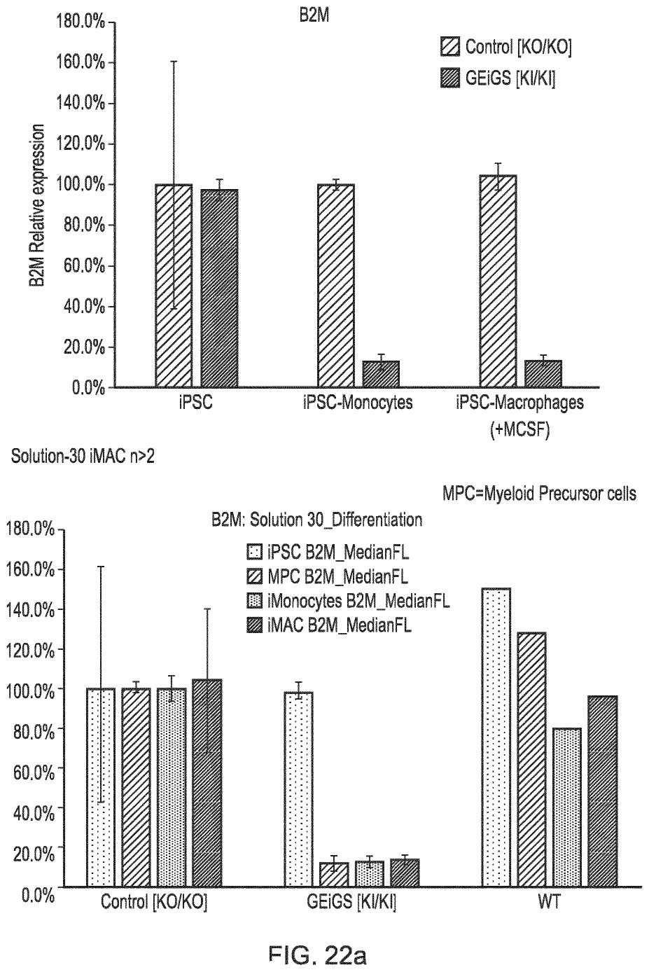

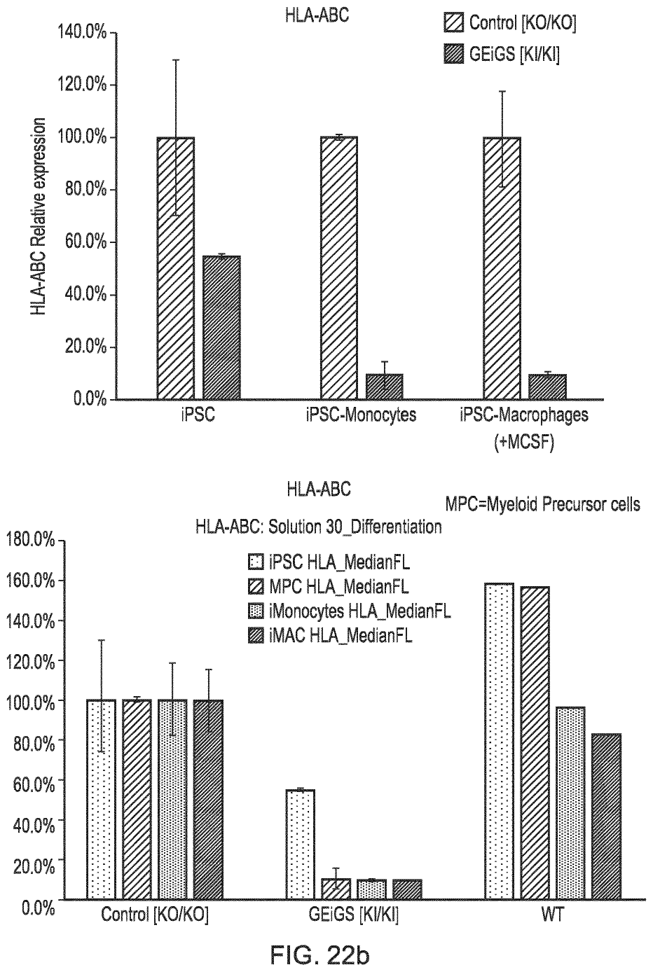

After several days differentiation in culture, cells were isolated for analysis of target MHC-I protein expression (B2M or HLA-ABC). The relative B2M and HLA-ABC expression in GEiGS modified iPSC, monocytes and macrophages (miR-21 redirected to target B2M, i.e. Solution 30) compared to control cells in which the endogenous miR-21 has been knocked out (KO/KO). The GEiGS and control lines were differentiated in vitro first to monocytes and subsequently to macrophages. B2M and HLA-ABC were assayed by flow cytometry showing effective silencing in myeloid cells (84-88% silencing), but not at the iPSC stage (FIG. 22).

The relative B2M protein expression levels between the Solution 30 undifferentiated iPSC and its undifferentiated homozygous KO control iPSC line were about the same indicating that disrupting the endogenous miR-21 locus does not produce an unspecific B2M silencing effect in the iPSC stage. Subsequently, when the iPSC were differentiated, the myeloid progenitors, monocytes and macrophages from the (wild type) control (unedited), homozygous miR-21 KO and the homozygous Solution 30 (KI/KI) cell lines were able to differentiate effectively and with the same efficiency, however the homozygous Solution 30 (KI/KI) cell line showed a specific reduction of B2M protein expression only at the stage of myeloid progenitors, monocytes and macrophages (FIG. 22a). This demonstrates that modifying the miR-21 locus to incorporate Solution 30 does not perturb the ability of the stem cells to differentiate into myeloid lineage cells and that B2M cell surface expression is specifically reduced only the myeloid lineage stage cells.

An antibody to cell surface expressed classical HLA-A/B/C indicated that, as predicted for B2M silencing via Solution 30, there was a reduced cell surface expression of HLA-A/B/C as B2M is required for the correct processing and cell surface distribution of MHC-I complex proteins.

This example indicates that silencing of B2M results in reduced cell surface expression of HLA-A/B/C and therefore MHC-I complex.

### Example 17—B2M Solutions Screen

A screen was carried out using validated solutions targeting B2M which have different predicted levels of silencing in activated T cells based on their known B2M silencing specificity on other human cell types (e.g. iPSC).

The purpose was to demonstrate the utility of Solutions delivery via a lentivirus (LV), as opposed to plasmid based delivery, into T cells, a notoriously difficult cell type to ectopically express Solutions in plasmid based systems. It was also used to validate a dual reporter system in which 3′ UTR sequences of the B2M transcript were placed contiguously with encoding sequences for copGFP. Screening for copGFP regression in cells expressing validated Solutions (Triggers) against the B2M sequence, rather than relying on detection of residual B2M protein expression on the T-cell surface using flow cytometry, was a useful test of this alternative analytical approach for Solutions' validation and supported more rapid identification of effective Solutions, as example intracellular transcription factor targets.

Effective Solutions are those with B2M silencing specificity as a result of binding with perfect complementarity to the cognate B2M target gene sequences.

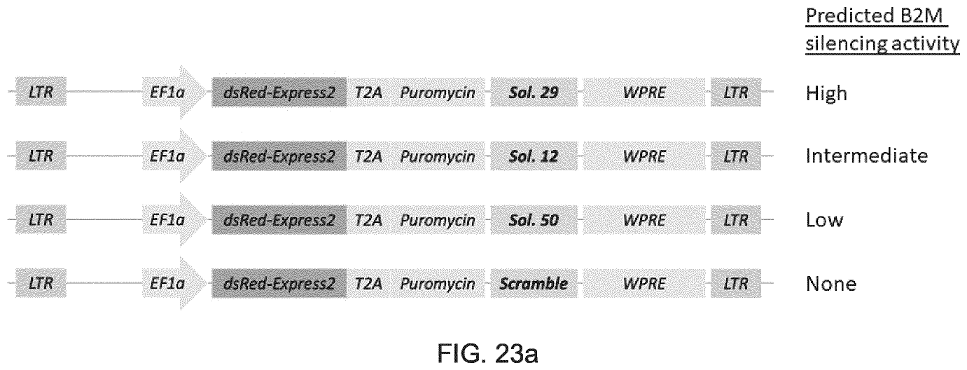

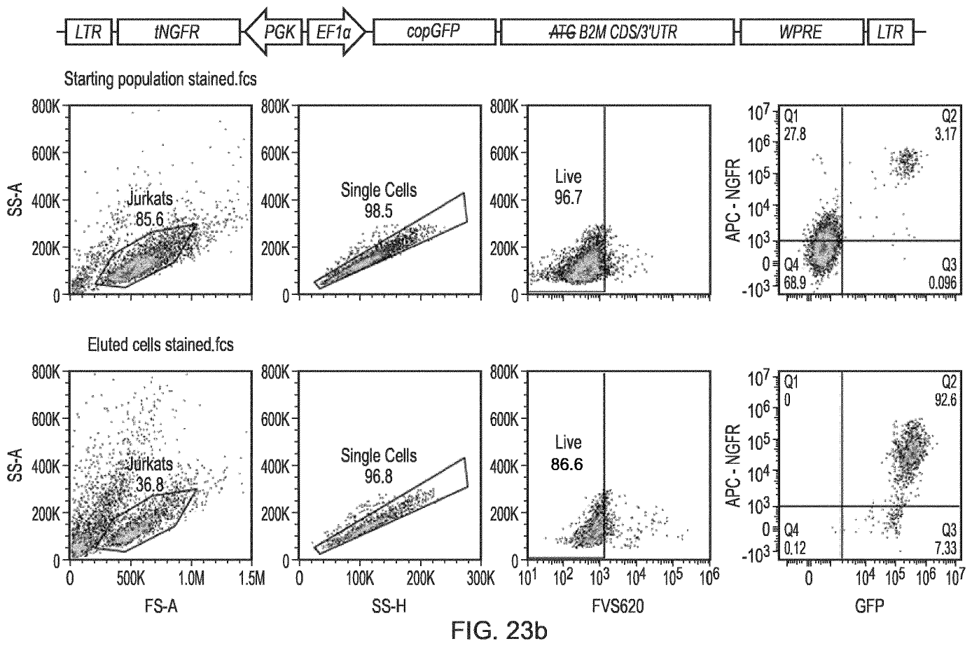

Constructs for the lentiviral (LV) delivery test and validation of the new reporter system for B2M silencing based on copGFP regression in T cells are described (FIG. 23a/b). Constructs with high (Solution 29), intermediate (Solution 12), low (Solution 50) and no (scrambled) B2M silencing activity are shown in FIG. 23a. The predictions of activity were based on previous effectiveness seen or predicted in Examples 2, 6 and 15 and FIGS. 10, 12 and 21 for Solution 29, Example 8 and FIG. 13 for Solution 12 and Example 2, FIG. 8 for Solution 50.

In order to validate LV-based Solution delivery, this reporter system was transduced into Jurkat cell lines. Jurkat cells were transduced with a pooled mixture of Solution 12, Solution 29, Solution 50 or Scramble (negative control) as individual LV constructs, to generate a B2M Jurkat reporter pool of cells. Distinct multiplicities of infection (MOI) were evaluated (1, 5, and 10) for each pool of LV constructs. dsRED expression from each Solution construct was used to monitor the appearance of and to set the gate for detection of Solution expressing cell pools. Cell surface detection of the low affinity NGF-receptor protein on cell surface was used to gate for cell pools expressing the copGFP sequence containing the B2M 3′UTR binding sequences for each Solution.

The gating strategy to identify those cells expressing both dsRED (Solution sequence) and copGFP is outlined in FIG. 23b. Levels of copGFP expression was monitored on day 3, 6 and 9 post transduction and verification of copGFP regression in B2M Jurkat reporter cells containing an effective Solution was confirmed using residual B2M cell surface expression in flow cytometry.

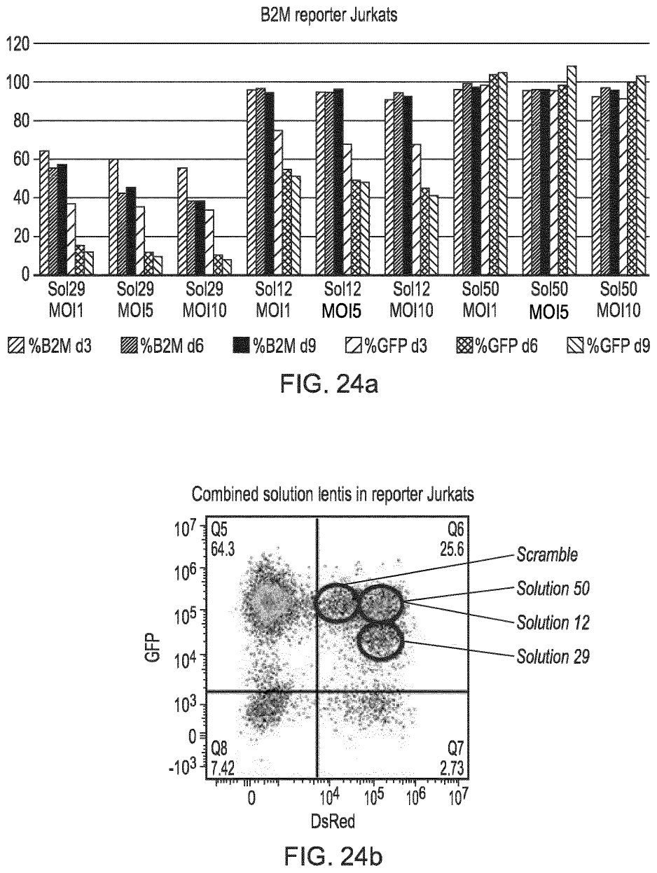

As shown in FIG. 24a, GFP regression as a result of transcript degradation due to Solution binding to the B2M 3′UTR in the construct displayed the predicted pattern of silencing specificity as being Solution 29>Solution 12>Solution 50. There was close correlation between the quantitative amount of copGFP regression and detectable amounts of residual B2M protein staining, with the silencing effect demonstrated by these disparate analytical approaches being both dose (MOI) and time dependent (days) following transduction.

When analysed in flow cytometry and gated for Solution expressing cells (high dsRED) and copGFP expression, the B2M reporter Jurkat pools displayed a pattern of B2M silencing Solution effect that closely mimics that predicted for each individual Solution if expressed alone, confirming that LV pool transduction of T-cells will be an effective alternative to plasmid based systems for further Solution design testing and validation (FIG. 24b).

This demonstrates that lentiviral Solution delivery can lead to successful knockdown of the target gene of interest as detectable by copGFP regression, and that different levels of target gene expression can be achieved using specifically designed Solutions.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

Esrick E B, Lehmann L E, Biffi A, Achebe M, Brendel C, Ciuculescu M F, Daley H, MacKinnon B, Morris E, Federico A, Abriss D, Boardman K, Khelladi R, Shaw K, Negre H, Negre O, Nikiforow S, Ritz J, Pai S-Y, London W B, Dansereau C, Heeney M M, Armant M, Manis J P, Williams D A. 2021. Post-Transcriptional Genetic Silencing of BCL11A to Treat Sickle Cell Disease. New England Journal of Medicine 384:205-215. Doi:10.1056/nejmoa2029392
Fellmann C, Hoffmann T, Sridhar V, Hopfgartner B, Muhar M, Roth M, Lai D Y, Barbosa I A M, Kwon J S, Guan Y, Sinha N, Zuber J. 2013. An Optimized microRNA Backbone for Effective Single-Copy RNAi. Cell Reports 5:1704-1713. Doi:10.1016/j.celrep.2013.11.020
Hacke K, Falahati R, Flebbe-Rehwaldt L, Kasahara N, Gaensler K M. Suppression of HLA expression by lentivirus-mediated gene transfer of siRNA cassettes and in vivo chemoselection to enhance hematopoietic stem cell transplantation. Immunol Res. 2009; 44(1-3):112-26. Doi: 10.1007/s12026-008-8088-z.
Haga K, Lemp N A, Logg C R, Nagashima J, Faure-Kumar E, Gomez G G, Kruse C A, Mendez R, Stripecke R, Kasahara N, Cicciarelli J C. Permanent, lowered HLA class I expression using lentivirus vectors with shRNA constructs: Averting cytotoxicity by alloreactive T lymphocytes. Transplant Proc. 2006 December; 38(10):3184-8. Doi: 10.1016/j.transproceed.2006.10.158.
Han X, Wang M, Duan S, Franco P J, Kenty J H, Hedrick P, Xia Y, Allen A, Ferreira L M R, Strominger J L, Melton D A, Meissner T B, Cowan C A. Generation of hypoimmunogenic human pluripotent stem cells. Proc Natl Acad Sci USA. 2019 May 21; 116(21):10441-10446. Doi: 10.1073/pnas.1902566116.
Karabekian Z, Ding H, Stybayeva G, Ivanova I, Muselimyan N, Haque A, Toma I, Posnack N G, Revzin A, Leitenberg D, Laflamme M A, Sarvazyan N. 2015. HLA Class I Depleted hESC as a Source of Hypoimmunogenic Cells for Tissue Engineering Applications. Tissue Eng Part A 21:2559-2571. Doi:10.1089/ten.tea.2015.0105
Wang P, Yigit M V, Ran C, Ross A, Wei L, Dai G, Medarova Z, Moore A. 2012. A Theranostic Small Interfering RNA Nanoprobe Protects Pancreatic Islet Grafts From Adoptively Transferred Immune Rejection. Diabetes 61:3247-3254. Doi:10.2337/db12-0441

1. An inhibitory RNA, or a polynucleotide sequence encoding an inhibitory RNA, preferably a microRNA, the inhibitory RNA comprising or consisting a sequence selected from:
(i) any one of SEQ ID Nos: 1-17, 64-127;
(ii) a variant of any one of SEQ ID Nos: 1-17, 64-127;
(iii) a fragment of any one of SEQ ID Nos: 1-17, 64-127; and
(iv) a DNA sequence complementary to any one of (i), (ii) or (iii).

2. The inhibitory RNA or polynucleotide sequence according to claim 1, comprising or consisting of a sequence selected from:
(i) any one of SEQ ID Nos: 18-34, 128-191;
(ii) a variant of any one of SEQ ID Nos: 18-34, 128-191;
(iii) a fragment of any one of SEQ ID Nos: 18-34, 128-191; and
(iv) a DNA sequence complementary to any one of (i), (ii) or (iii).

3. The polynucleotide sequence encoding an inhibitory RNA according to claim 1 or 2, comprising or consisting a sequence selected from:
(i) any one of SEQ ID Nos: 35-51;
(ii) a variant of any one of SEQ ID Nos: 35-51; and
(iii) a fragment of any one of SEQ ID Nos: 35-51.

4. The inhibitory RNA or polynucleotide sequence encoding an inhibitory RNA of any one of claims 1-3, which is RNA.

5. The inhibitory RNA or polynucleotide sequence encoding an inhibitory RNA of any one of claims 1-3, which is DNA.

6. The inhibitory RNA or a polynucleotide sequence encoding an inhibitory RNA according to any preceding claim, which is a synthetic nucleic acid sequence.

7. The inhibitory RNA or polynucleotide sequence encoding an inhibitory RNA according to any preceding claim, which comprises or encodes a microRNA scaffold.

8. The inhibitory RNA or polynucleotide sequence encoding an inhibitory RNA according to claim 7, wherein the microRNA scaffold is a human pre-microRNA scaffold.

9. The inhibitory RNA or polynucleotide sequence encoding an inhibitory RNA according to claim 8, wherein the human pre-microRNA scaffold is selected from any one of:
(i) hsa-mir-191; (ii) hsa-mir-302a; (iii) hsa-mir-302c; (iv) hsa-mir-93; (v) hsa-mir-106a; (vi) hsa-mir-106b; (vii) hsa-mir-200c; (viii) hsa-mir-20a; (iv) hsa-mir-21; (x) hsa-mir-363; (xi) hsa-mir-518b; (xii) hsa-mir-744; (xiii) hsa-mir-99b; (xiv) hsa-mir-320a; (xv) hsa-mir-520f; (xvi) hsa-mir-652; (xvi) hsa-mir-1180; (xvii) hsa-mir-15b; (xviii) hsa-mir-182; (xix) hsa-mir-23a; (xx) hsa-mir-26b; (xxi) hsa-mir-335; (xxii) hsa-mir-361; (xxiii) hsa-mir-1307; (xxiv) hsa-mir-205; (xxv) hsa-mir-22; (xxvi) hsa-mir-221; (xxvii) hsa-mir-222; (xxviii) hsa-mir-30e; (xxix) hsa-mir-423; (xxx) hsa-mir-519c; (xxxi) hsa-mir-92b; (xxxii) hsa-mir-30; (xxxiii) hsa-mir-302b; (xxxiv) hsa-mir-375; (xxxv) hsa-let-7b; (xxxv) hsa-mir-98; and (xxxvi) hsa-let-7g.

10. The inhibitory RNA or polynucleotide sequence encoding an inhibitory RNA according to any preceding claim, wherein the polynucleotide of any one of SEQ ID NOs: 1-17, 64-127 is inserted into a human pre-microRNA sequence, preferably wherein:
(i) SEQ ID NO: 1 or 7 is inserted into hsa-mir-191 (SEQ ID NO: 18 or 24, respectively);
(ii) SEQ ID NO: 2 or 11 is inserted into hsa-mir-302a (SEQ ID NO: 19 or 28, respectively);
(iii) SEQ ID NO: 3 or 12 is inserted into hsa-mir-302c (SEQ ID NO: 20 or 29, respectively);
(iv) SEQ ID NO: 4 or 16 is inserted into hsa-mir-93 (SEQ ID NO: 21 or 33, respectively);
(v) SEQ ID NO: 5 is inserted into hsa-mir-106a (SEQ ID NO: 22);
(vi) SEQ ID NO: 6 is inserted into hsa-mir-106b (SEQ ID NO: 23);
(vii) SEQ ID NO: 8 is inserted into hsa-mir-200c (SEQ ID NO: 25);
(viii) SEQ ID NO: 9 is inserted into hsa-mir-20a (SEQ ID NO: 26);
(iv) SEQ ID NO: 10 is inserted into hsa-mir-21 (SEQ ID NO: 27);
(x) SEQ ID NO: 13 is inserted into hsa-mir-363 (SEQ ID NO: 30);
(xi) SEQ ID NO: 14 is inserted into hsa-mir-518b (SEQ ID NO: 31);
(xii) SEQ ID NO: 15 is inserted into hsa-mir-744 (SEQ ID NO: 32);
(xiii) SEQ ID NO: 17 is inserted into hsa-mir-99b (SEQ ID NO: 34);
(xiv) SEQ ID NO: 64, SEQ ID NO: 73 or SEQ ID NO: 89 is inserted into hsa-mir-320a (SEQ ID NO: 128, SEQ ID NO: 137 or SEQ ID NO: 153 respectively);
(xv) SEQ ID NO: 65, SEQ ID NO: 77 or SEQ ID NO: 94 is inserted into hsa-mir-520f (SEQ ID NO: 129, SEQ ID NO: 141 or SEQ ID NO: 158 respectively);
(xvi) SEQ ID NO: 66, SEQ ID NO: 78 or SEQ ID NO: 95 is inserted into hsa-mir-652 (SEQ ID NO: 130, SEQ ID NO: 142 or SEQ ID NO: 159 respectively);
(xvii) SEQ ID NO: 67 is inserted into hsa-mir-106a (SEQ ID NO: 131);
(xviii) SEQ ID NO: 68 or SEQ ID NO: 79 is inserted into hsa-mir-1180 (SEQ ID NO: 132 or SEQ ID NO: 143 respectively);
(xix) SEQ ID NO: 69 or SEQ ID NO: 81 is inserted into hsa-mir-15b (SEQ ID NO: 133 or SEQ ID NO: 145 respectively);
(xx) SEQ ID NO: 70, SEQ ID NO: 82 or SEQ ID NO: 99 is inserted into hsa-mir-182 (SEQ ID NO: 134, SEQ ID NO: 146 or SEQ ID NO: 163 respectively);
(xxi) SEQ ID NO: 71 or SEQ ID NO: 87 is inserted into hsa-mir-23a (SEQ ID NO:135 or SEQ ID NO: 151 respectively);
(xxii) SEQ ID NO: 72 is inserted into hsa-mir-26b (SEQ ID NO: 136);
(xxiii) SEQ ID NO: 74 or SEQ ID NO: 90 is inserted into hsa-mir-335 (SEQ ID NO: 138 or SEQ ID NO: 154 respectively);
(xxiv) SEQ ID NO: 75 or SEQ ID NO: 91 is inserted into hsa-mir-361 (SEQ ID NO: 139 or SEQ ID NO: 155 respectively);
(xxv) SEQ ID NO: 76 is inserted into hsa-mir-518b (SEQ ID NO: 140);
(xxvi) SEQ ID NO: 80 is inserted into hsa-mir-1307 (SEQ ID NO: 144);
(xxvii) SEQ ID NO: 83 is inserted into hsa-mir-205 (SEQ ID NO: 147);
(xxviii) SEQ ID NO: 84 is inserted into hsa-mir-22 (SEQ ID NO: 148);
(xxix) SEQ ID NO: 85 is inserted into hsa-mir-221 (SEQ ID NO: 149);
(xxx) SEQ ID NO: 86 is inserted into hsa-mir-222 (SEQ ID NO: 150);
(xxxi) SEQ ID NO: 88 is inserted into hsa-mir-30e (SEQ ID NO: 152);
(xxxii) SEQ ID NO: 92 is inserted into hsa-mir-423 (SEQ ID NO: 156);
(xxxiii) SEQ ID NO: 93 is inserted into hsa-mir-519c (SEQ ID NO: 157);
(xxxiv) SEQ ID NO: 96 is inserted into hsa-mir-92b (SEQ ID NO: 160);
(xxxv) SEQ ID NO: 97, SEQ ID NO: 103, SEQ ID NO: 104 or SEQ ID NO: 109 is inserted into hsa-mir-302a (SEQ ID NO: 161, SEQ ID NO: 167, SEQ ID NO: 168 or SEQ ID NO: 173 respectively);
(xxxvi) SEQ ID NO: 98, SEQ ID NO: 107, SEQ ID NO: 108 or SEQ ID NO: 111 is inserted into hsa-mir-302c (SEQ ID NO: 162, SEQ ID NO: 171, SEQ ID NO: 172 or SEQ ID NO: 175 respectively);
(xxxvii) SEQ ID NO: 100 is inserted into hsa-mir-191 (SEQ ID NO: 164);
(xxxviii) SEQ ID NO: 101 or SEQ ID NO: 102 is inserted into hsa-mir-30 (SEQ ID NO:
165 or SEQ ID NO: 166 respectively);
(xxxix) SEQ ID NO: 105, SEQ ID NO: 106 or SEQ ID NO: 110 is inserted into hsa-mir-302b (SEQ ID NO: 169, SEQ ID NO: 170 or SEQ ID NO: 174 respectively);
(xxxx) SEQ ID NO: 112, SEQ ID NO: 113 or SEQ ID NO: 114 is inserted into hsa-mir-375 (SEQ ID NO: 176, SEQ ID NO: 177 or SEQ ID NO: 178 respectively);
(xxxxi) SEQ ID NO: 115, SEQ ID NO: 119, SEQ ID NO: 122 or SEQ ID NO: 125 is inserted into hsa-let-7b (SEQ ID NO: 179, SEQ ID NO: 183, SEQ ID NO: 186 or SEQ ID NO: 189 respectively);
(xxxxii) SEQ ID NO: 116, SEQ ID NO: 120, SEQ ID NO: 123 or SEQ ID NO: 126 is inserted into hsa-let-7g (SEQ ID NO: 180, SEQ ID NO: 184, SEQ ID NO: 187 or SEQ ID NO: 190 respectively); and/or
(xxxxiii) SEQ ID NO: 117, SEQ ID NO: 118, SEQ ID NO: 121, SEQ ID NO: 124 or SEQ ID NO: 127 is inserted into hsa-mir-98 (SEQ ID NO: 181, SEQ ID NO: 182, SEQ ID NO: 185, SEQ ID NO: 188 or SEQ ID NO: 191 respectively).

11. The polynucleotide sequence encoding an inhibitory RNA according to any preceding claim, which comprises a genomic sequence flanking both sides of a human pre-microRNA suitably wherein the polynucleotide sequence comprises:
(i) SEQ ID NO: 35 (which comprises SEQ ID NO: 2 or SEQ ID NO: 19);
(ii) SEQ ID NO: 36 (which comprises SEQ ID NO: 11 or SEQ ID NO: 28);
(iii) SEQ ID NO: 37 (which comprises SEQ ID NO: 3 or SEQ ID NO: 20);
(iv) SEQ ID NO: 38 (which comprises SEQ ID NO: 12 or SEQ ID NO: 29);
(v) SEQ ID NO: 39 (which comprises SEQ ID NO: 9 or SEQ ID NO: 26);
(vi) SEQ ID NO: 40 (which comprises SEQ ID NO: 8 or SEQ ID NO: 25);
(vii) SEQ ID NO: 41 (which comprises SEQ ID NO: 10 or SEQ ID NO: 27);
(viii) SEQ ID NO: 42 (which comprises SEQ ID NO: 13 or SEQ ID NO: 30);
(iv) SEQ ID NO: 43 (which comprises SEQ ID NO: 4 or SEQ ID NO: 21);
(x) SEQ ID NO: 44 (which comprises SEQ ID NO: 16 or SEQ ID NO: 33);
(xi) SEQ ID NO: 45 (which comprises SEQ ID NO: 5 or SEQ ID NO: 22;
(xii) SEQ ID NO: 46 (which comprises SEQ ID NO: 6 or SEQ ID NO: 23;
(xiii) SEQ ID NO: 47 (which comprises SEQ ID NO: 1 or SEQ ID NO: 18;
(xvi) SEQ ID NO: 48 (which comprises SEQ ID NO: 7 or SEQ ID NO: 24;
(xv) SEQ ID NO: 49 (which comprises SEQ ID NO: 15 or SEQ ID NO: 32;
(xvi) SEQ ID NO: 50 (which comprises SEQ ID NO: 14 or SEQ ID NO: 31; and/or
(xvii) SEQ ID NO: 51 (which comprises SEQ ID NO: 17 or SEQ ID NO: 34).

12. The inhibitory RNA or polynucleotide sequence encoding an inhibitory RNA according to any one of claims 1, 2, 4 or 6-10, wherein the inhibitory RNA targets the B2M RNA, suitably an exon sequence comprised in the messenger RNA from the B2M gene, suitably exon 2-1, 2-2 or exon 4, preferably wherein the inhibitory RNA comprises:
(i) SEQ ID NO: 2 or SEQ ID NO: 19, which targets B2M gene exon 2-2;
(ii) SEQ ID NO: 11 or SEQ ID NO: 28, which targets B2M gene exon 4;
(iii) SEQ ID NO: 3 or SEQ ID NO: 20, which targets B2M gene exon 2-2;
(iv) SEQ ID NO: 12 or SEQ ID NO: 29, which targets B2M gene exon 4;
(v) SEQ ID NO: 9 or SEQ ID NO: 26, which targets B2M gene exon 4;
(vi) SEQ ID NO: 8 or SEQ ID NO: 25, which targets B2M gene exon 4;
(vii) SEQ ID NO: 10 or SEQ ID NO: 27, which targets B2M gene exon 4;
(viii) SEQ ID NO: 13 or SEQ ID NO: 30, which targets B2M gene exon 4;
(iv) SEQ ID NO: 4 or SEQ ID NO: 21, which targets B2M gene exon 2-2;
(x) SEQ ID NO: 16 or SEQ ID NO: 33, which targets B2M gene exon 4;
(xi) SEQ ID NO: 5 or SEQ ID NO: 22, which targets B2M gene exon 4;
(xii) SEQ ID NO: 6 or SEQ ID NO: 23, which targets B2M gene exon 4;
(xiii) SEQ ID NO: 1 or SEQ ID NO: 18, which targets B2M gene exon 2-2;
(xvi) SEQ ID NO: 7 or SEQ ID NO: 24, which targets B2M gene exon 4;
(xv) SEQ ID NO: 15 or SEQ ID NO: 32, which targets B2M gene exon 4;
(xvi) SEQ ID NO: 14 or SEQ ID NO: 31, which targets B2M gene exon 4;
(xvii) SEQ ID NO: 17 or SEQ ID NO: 34, which targets B2M gene exon 4;
(xviii) SEQ ID NOs: 64-66 or SEQ ID NOs: 128-130, which target B2M exon 2-1;
(xix) SEQ ID NOs: 67-78 or SEQ ID NOs: 129-142, which target B2M exon 2-2; and/or
(xx) SEQ ID NOs: 79-96 or SEQ ID NOs: 143-160, which target B2M exon 4.

13. An expression cassette comprising a polynucleotide sequence encoding an inhibitory RNA according to any one of claims 1-12 operably linked to one or more regulatory elements suitable for permitting transcription of the inhibitory RNA in a cell, preferably a promoter, optionally a constitutive promoter or an inducible promoter.

14. The expression cassette according to claim 13 wherein the promoter is EF1alpha.

15. A vector comprising a polynucleotide sequence encoding an inhibitory RNA of any one of claims 1-12 or an expression cassette according to any one of claim 13 or 14.

16. The vector according to claim 15, which comprises DNA encoding regulatory elements suitable for permitting transcription of the inhibitory RNA in a cell, preferably a promoter, optionally a constitutive promoter or an inducible promoter.

17. The vector according to claim 16, wherein in the cell is a mammalian cell, preferably a human cell.

18. The vector according to any one of claims 15-17, which is a viral vector.

19. The vector according to claim 18, which is a lentiviral, adenoviral, adeno-associated virus (AAV), retroviral, alphavirus, herpes virus, arena virus, measles virus, poxvirus or paramyxovirus vector.

20. The vector according to any one of claims 15-17, which is a plasmid.

21. The vector according to claim 20, which is plasmid VB210602-1567ytv.

22. A virion comprising the vector of any one of claims 15-21.

23. A synthetic microRNA comprising or consisting of a sequence selected from any one of SEQ ID Nos: 1-17, 64-127 or functional variants or fragments thereof.

24. A synthetic pre-microRNA comprising or consisting of a sequence selected from any one of SEQ ID Nos: 18-34, 128-191 or functional variants or fragments thereof.

25. The synthetic microRNA or synthetic pre-microRNA according to claim 23 or 24, which is a modified endogenous microRNA or pre-microRNA molecule.

26. A synthetic microRNA adapted to target a messenger RNA transcribed from the B2M gene, the synthetic microRNA comprising a sequence according to any one of SEQ ID Nos: 1-17, 64-127 or a functional variant or fragment thereof, optionally wherein the synthetic microRNA comprises a sequence according to any one of SEQ ID NOs: 18-34, 128-191 or a functional variant or fragment thereof.

27. A cell comprising or expressing the synthetic microRNA according to any one of claims 23-26, wherein the cell is modified to express the synthetic microRNA such that the synthetic microRNA targets a messenger RNA transcribed from the B2M gene.

28. The cell according to claim 27, wherein the synthetic microRNA degrades the messenger RNA transcribed from the B2M gene, preferably wherein the messenger RNA is partially silenced and B2M protein expression is modified in a range of 10%-90% expression compared to a control cell.

29. A cell comprising or expressing a sequence encoding a synthetic inhibitory RNA, preferably a synthetic microRNA, wherein the synthetic inhibitory RNA is adapted to target B2M.

30. The cell according to claim 29, wherein the sequence encoding the synthetic inhibitory RNA, preferably a synthetic microRNA, encodes one or more of SEQ ID NOs: 1-17, 64-127.

31. The cell according to claim 29, wherein the sequence encoding the synthetic microRNA encodes one or more of SEQ ID NOs: 18-34, 128-191.

32. The cell according to claim 29, wherein the sequence encoding the synthetic microRNA comprises one or more of SEQ ID NOs: 35-51.

33. The cell according to any one of claims 29-32, wherein the sequence encoding the synthetic inhibitory RNA, preferably microRNA, is genomic or episomal, preferably genomic.

34. The cell according to any one of claims 29-33, wherein the sequence encoding the synthetic microRNA is a genomic sequence encoding a modified endogenous microRNA, suitably wherein the cell is a human cell and the modified genomic sequence encodes a modified form of one of the following endogenous microRNAs:
(i) hsa-mir-191, optionally wherein hsa-mir-191 has been modified to comprise SEQ ID NO: 1, SEQ ID NO: 7 or SEQ ID NO: 100;
(ii) hsa-mir-302a, optionally wherein hsa-mir-302a has been modified to comprise SEQ ID NO: 2, SEQ ID NO: 11, SEQ ID NO: 97, SEQ ID NO: 103, SEQ ID NO: 104 or SEQ ID NO: 109;
(iii) hsa-mir-302c, optionally wherein hsa-mir-302c has been modified to comprise SEQ ID NO: 3, SEQ ID NO: 12, SEQ ID NO: 98, SEQ ID NO: 107, SEQ ID NO: 108 or SEQ ID NO: 111;
(iv) hsa-mir-93, optionally wherein hsa-mir-93 has been modified to comprise SEQ ID NO: 4 or 16;
(v) hsa-mir-106a, optionally wherein hsa-mir-106a has been modified to comprise SEQ ID NO: 5 or SEQ ID NO: 67;
(vi) hsa-mir-106b, optionally wherein hsa-mir-106b has been modified to comprise SEQ ID NO: 6;
(vii) hsa-mir-200c, optionally wherein hsa-mir-200c has been modified to comprise SEQ ID NO: 8;
(viii) hsa-mir-20a, optionally wherein hsa-mir-20a has been modified to comprise SEQ ID NO: 9;
(iv) hsa-mir-21, optionally wherein hsa-mir-21 has been modified to comprise SEQ ID NO: 10;
(x) hsa-mir-363, optionally wherein hsa-mir-363 has been modified to comprise SEQ ID NO: 13;
(xi) hsa-mir-518b, optionally wherein hsa-mir-518b has been modified to comprise SEQ ID NO: 14 or SEQ ID NO: 76;
(xii) hsa-mir-744, optionally wherein hsa-mir-744 has been modified to comprise SEQ ID NO: 15;
(xiii) hsa-mir-99b, optionally wherein hsa-mir-99b has been modified to comprise SEQ ID NO: 17;
(xiv) hsa-mir-320a, optionally wherein hsa-mir-320a has been modified to comprise SEQ ID NO: 64, SEQ ID NO: 73 or SEQ ID NO: 89;
(xv) hsa-mir-520f, optionally wherein hsa-mir-520f has been modified to comprise SEQ ID NO: 65, SEQ ID NO: 77 or SEQ ID NO: 94;
(xvi) hsa-mir-652, optionally wherein hsa-mir-652 has been modified to comprise SEQ ID NO: 66, SEQ ID NO: 78 or SEQ ID NO: 95;
(xvii) hsa-mir-1180, optionally wherein hsa-mir-1180 has been modified to comprise SEQ ID NO: 68 or SEQ ID NO: 79;
(xviii) hsa-mir-15b, optionally wherein hsa-mir-15b has been modified to comprise SEQ ID NO: 69 or SEQ ID NO: 81;
(xix) hsa-mir-182, optionally wherein hsa-mir-182 has been modified to comprise SEQ ID NO: 70, SEQ ID NO: 82 or SEQ ID NO: 99;
(xx) hsa-mir-23a, optionally wherein hsa-mir-23a has been modified to comprise SEQ ID NO: 71 or SEQ ID NO: 87;
(xxi) hsa-mir-26b, optionally wherein hsa-mir-26b has been modified to comprise SEQ ID NO: 72;
(xxii) hsa-mir-335, optionally wherein hsa-mir-335 has been modified to comprise SEQ ID NO: 74 or SEQ ID NO: 90;
(xxiii) hsa-mir-361, optionally wherein hsa-mir-361 has been modified to comprise SEQ ID NO: 75 or SEQ ID NO: 91;
(xxiv) hsa-mir-1307, optionally wherein hsa-mir-1307 has been modified to comprise SEQ ID NO: 80;
(xxv) hsa-mir-205, optionally wherein hsa-mir-205 has been modified to comprise SEQ ID NO: 83;
(xxvi) hsa-mir-22, optionally wherein hsa-mir-22 has been modified to comprise SEQ ID NO: 84;
(xxvii) hsa-mir-221, optionally wherein hsa-mir-221 has been modified to comprise SEQ ID NO: 85;
(xxviii) hsa-mir-222, optionally wherein hsa-mir-222 has been modified to comprise SEQ ID NO: 86;
(xxix) hsa-mir-30e, optionally wherein hsa-mir-30e has been modified to comprise SEQ ID NO: 88;
(xxx) hsa-mir-423, optionally wherein hsa-mir-423 has been modified to comprise SEQ ID NO: 92;
(xxxi) hsa-mir-519c, optionally wherein hsa-mir-519c has ben modified to comprise SEQ ID NO: 93;
(xxxii) hsa-mir-92b, optionally wherein hsa-mir-92b has been modified to comprise SEQ ID NO: 96;
(xxxiii) hsa-mir-30, optionally wherein hsa-mir-30 has been modified to comprise SEQ ID NO: 101 or SEQ ID NO: 102;
(xxxiv) hsa-mir-302b, optionally wherein hsa-mir-302b has been modified to comprise SEQ ID NO: 105, SEQ ID NO: 106 or SEQ ID NO: 110
(xxxv) hsa-mir-375, optionally wherein hsa-mir-375 has been modified to comprise SEQ ID NO: 112, SEQ ID NO: 113 or SEQ ID NO: 114;
(xxxvi) hsa-let-7b, optionally wherein hsa-let-7b has been modified to comprise SEQ ID NO: 115, SEQ ID NO: 119, SEQ ID NO: 122 or SEQ ID NO: 125;
(xxxvii) hsa-let-7g, optionally wherein hsa-let-7g has been modified to comprise SEQ ID NO: 116, SEQ ID NO: 120, SEQ ID NO: 123 or SEQ ID NO: 126; and
(xxxviii) hsa-mir-98, optionally wherein hsa-mir-98 has been modified to comprise SEQ ID NO: 117, SEQ ID NO: 118, SEQ ID NO: 121, SEQ ID NO: 124 or SEQ ID NO: 127.

35. The cell according to any one of claims 29-34, wherein the cell exhibits reduced B2M expression, preferably not completely eliminated B2M expression, suitably wherein B2M is expressed at a level of from 1% to 90%, 5%-90% or 10%-90% compared to a control cell.

36. The cell according to any one of claims 29-35, wherein the cell is de-targeted from the innate and adaptive immune systems.

37. The cell according to any one of claims 29-36, wherein the cell is a eukaryotic cell, preferably a mammalian cell, more preferably a human cell.

38. The cell according to claim any one of claims 29-37, wherein the cell is an induced pluripotent stem cell or a cell derived through differentiation of an induced pluripotent stem cell.

39. The cell according to any one of claims 29-38, wherein the cell is a pancreatic beta cell or an immune cell.

40. A cell comprising the expression cassette of any one of claim 13 or 14, vector of any one of claims 15-21, virion of claim 22 or synthetic microRNA or pre-microRNA of claims 23-25.

41. A composition comprising the cell of any one of claims 29-40.

42. An inhibitory RNA or polynucleotide encoding an inhibitory RNA according to any one of claims 1-12, an expression cassette according to any one of claim 13 or 14, a vector according to any one of claims 15-21, a virion according to claim 22, a synthetic microRNA or pre-microRNA according to any one of claims of claim 23-26, a cell according to any one of claims 27-40 or a composition according to claim 41 for use in therapy.

43. An inhibitory RNA or polynucleotide encoding an inhibitory RNA according to any one of claims 1-12, an expression cassette according to any one of claim 13 or 14, a vector according to any one of claims 15-21, a virion according to claim 22, a synthetic microRNA or pre-microRNA according to any one of claims of claim 23-26, a cell according to any one of claims 27-40 or a composition according to claim 41 for use in a method of treating an autoimmune disease or cancer in a subject.

44. An inhibitory RNA or polynucleotide encoding an inhibitory RNA, an expression cassette, a vector, a virion or a synthetic microRNA or pre-microRNA according to claim 43, wherein the autoimmune disease is preferably Type I diabetes.

45. An inhibitory RNA or polynucleotide encoding an inhibitory RNA according to any one of claims 1-12, an expression cassette according to any one of claim 13 or 14, a vector according to any one of claims 15-21, a virion according to claim 22 or a synthetic microRNA or pre-microRNA according to any one of claims of claim 23-26, for use in a method of degrading the messenger RNA transcribed from the B2M gene in a cell, preferably wherein the messenger RNA is partially silenced and B2M protein expression is modified in a range of 10%-90% expression compared to a control cell.

46. An inhibitory RNA or polynucleotide encoding an inhibitory RNA, an expression cassette, a vector, a virion or a synthetic microRNA or pre-microRNA for use according to claim 45, wherein the cell is subsequently transferred into a mammal, preferably a human, to treat an autoimmune disease or cancer, preferably wherein the autoimmune disease is Type I diabetes.

47. A cell according to any one of claims 27-40 or a composition according to claim 41 for use in a method of treating an autoimmune disease or cancer in a subject, wherein the method comprises administering the cell or composition in a therapeutic amount to the subject.

48. The cell or composition for use according to claim 47, wherein the autoimmune disease is Type I diabetes.

49. A method of treating an autoimmune disease or cancer in a subject, preferably wherein the autoimmune disease is Type I diabetes, wherein the method comprises administering the cell or composition in a therapeutic amount to the subject.

50. Use of a cell according to any one of claims 27-40 or a composition according to claim 41 in the manufacture of a medicament for treating an autoimmune disease or cancer in a subject, preferably wherein the autoimmune disease is Type I diabetes.

51. A method of modifying the expression of a protein in a cell comprising introducing into a cell an inhibitory RNA or polynucleotide encoding an inhibitory RNA according to any one of claims 1-12, an expression cassette according to any one of claim 13 or 14, a vector according to any one of claims 15-21, a virion according to claim 22 or a synthetic microRNA or pre-microRNA according to any one of claims of claim 23-26.

52. A method of modifying the translation of a messenger RNA transcript derived from a protein coding gene and subsequent expression of a protein in a cell comprising introducing into the cell an inhibitory RNA or polynucleotide encoding an inhibitory RNA according to any one of claims 1-12, an expression cassette according to any one of claim 13 or 14, a vector according to any one of claims 15-21, a virion according to claim 22 or a synthetic microRNA or pre-microRNA according to any one of claims of claim 23-26.

53. A method of modifying the translation of a messenger RNA transcript derived from a protein coding gene and subsequent expression of a protein in a cell comprising introducing into the cell a DNA editing agent conferring a silencing specificity of a microRNA towards a target RNA of interest, wherein the microRNA is modified such that the sequence of the microRNA comprises any one of SEQ ID NOs: 1-17, 64-127 thereby modifying the translation of the messenger RNA transcript into a protein molecule.

54. The method according to any one of claims 51-53, wherein the cell is a eukaryotic cell, preferably a mammalian cell, more preferably a human cell.

55. The method of claim 54 wherein the cell is a human cell and the method comprises editing the sequence of an endogenous micro RNA selected from the group consisting of: hsa-mir-191; hsa-mir-302a; hsa-mir-302c; hsa-mir-93; hsa-mir-106a; hsa-mir-106b; hsa-mir-200c; hsa-mir-20a; hsa-mir-21; hsa-mir-363; hsa-mir-518b; hsa-mir-744; hsa-mir-99b; hsa-mir-320a; hsa-mir-520f; hsa-mir-652; hsa-mir-1180; hsa-mir-15b; hsa-mir-182; hsa-mir-23a; hsa-mir-26b; hsa-mir-335; hsa-mir-361; hsa-mir-1307; hsa-mir-205; hsa-mir-22; hsa-mir-221; hsa-mir-222; hsa-mir-30e; hsa-mir-423; hsa-mir-519c; hsa-mir-92b; hsa-mir-30; hsa-mir-302b; hsa-mir-375; hsa-let-7b; has-let-7g; and hsa-mir-98.

56. The method according to any one of claims 51-55, wherein the cell is an induced pluripotent stem cell or a cell derived through differentiation of an induced pluripotent stem cell.

57. The method according to any one of claims 51-56, wherein the cell is a pancreatic beta cell or an immune cell.

58. The method according to any one of claims 51-57, wherein the protein is B2M or the messenger RNA transcript derived from a protein coding gene encodes B2M protein.

59. The method according to any one of claims 51-58, wherein the expression of the protein in the cell is modified in a range of 10%-90% when compared to a control cell.

60. The method according to any one of claims 53-59, wherein the DNA editing agent comprises a DNA editing system, preferably wherein the DNA editing system comprises a meganuclease, a zinc finger nucleases (ZFN), a transcription-activator like effector nuclease (TALEN), homology directed repair (HDR), CRISPR-endonuclease, dCRISPR-endonuclease, or a homing endonuclease.

61. The method according to any one of claims 53-60, wherein the DNA editing agent does not comprise an endonuclease.

62. The method according to any one of claims 53-61, wherein the DNA editing agent comprises an endonuclease.

63. The method according to claim 62, wherein the endonuclease comprises Cas9.

64. The method according to any one of claims 61-63, wherein the endonuclease comprises a catalytically inactive endonuclease.

65. The method of any one of claims 53-64, wherein the DNA editing agent is linked to a reporter for monitoring expression in the cell.

66. A cell or cell population obtained from the method of any one of claims 51-65.

67. A cell or cell population comprising a modified endogenous miRNA obtained by the method of any one of claims 51-66.

68. A composition comprising the cell or cell population of any one of claims 65-67.

69. The composition according to claim 68 for use in a cell therapy.
